SEQUENCE LISTING (342 sequences)

SEQ ID NO: 1 - protein, 22 aa, Artificial Sequence
  Synthetic peptide
NPEDCRQDPEANKSPEECKKLK

SEQ ID NO: 2 - protein, 22 aa, Artificial Sequence
  Synthetic peptide
  MISC_FEATURE (1)..(22): D-amino acid
NPEDCRQDPEANKSPEECKKLK

SEQ ID NO: 3 - protein, 26 aa, Artificial Sequence
  Synthetic peptide
HDPEKRKECEKKYTDPKKREECKRKA

SEQ ID NO: 4 - protein, 26 aa, Artificial Sequence
  Synthetic peptide
  misc (1)..(26): D-amino acid
HDPEKRKECEKKYTDPKKREECKRKA

SEQ ID NO: 5 - protein, 18 aa, Artificial Sequence
  Synthetic peptide
  MISC_FEATURE (9)..(9): D-amino acid
  MISC_FEATURE (18)..(18): D-amino acid
PVTWCVRIPPTVRCTVRP

SEQ ID NO: 6 - protein, 18 aa, Artificial Sequence
  Synthetic peptide
  MISC_FEATURE (1)..(8): D-amino acid
  MISC_FEATURE (10)..(17): D-amino acid
PVTWCVRIPPTVRCTVRP

SEQ ID NO: 7 - protein, 18 aa, Artificial Sequence
  Synthetic peptide
  MISC_FEATURE (9)..(9): D-amino acid
  MISC_FEATURE (18)..(18): D-amino acid
PVTWCVRIPPTVRCTVRD

SEQ ID NO: 8 - protein, 18 aa, Artificial Sequence
  Synthetic peptide
  MISC_FEATURE (1)..(8): D-amino acid
  MISC_FEATURE (10)..(17): D-amino acid
PVTWCVRIPPTVRCTVRD

SEQ ID NO: 9 - protein, 26 aa, Artificial Sequence
  Synthetic peptide
  MISC_FEATURE (12)..(12): D-amino acid
  MISC_FEATURE (15)..(24): D-amino acid
NPELQRKCKELDTRPEAERKCREESD

SEQ ID NO: 10 - protein, 26 aa, Artificial Sequence
  Synthetic peptide
  MISC_FEATURE (1)..(11): D-amino acid
  MISC_FEATURE (13)..(14): D-amino acid
  MISC_FEATURE (25)..(26): D-amino acid
NPELQRKCKELDTRPEAERKCREESD

SEQ ID NO: 11 - protein, 26 aa, Artificial Sequence
  Synthetic peptide
  MISC_FEATURE (6)..(6): D-amino acid
  MISC_FEATURE (20)..(20): D-amino acid
CQTWRRVSPEECRKYKEEYNCVRCTE

SEQ ID NO: 12 - protein, 26 aa, Artificial Sequence
  Synthetic peptide
  MISC_FEATURE (1)..(5): D-amino acid
  MISC_FEATURE (7)..(19): D-amino acid
  MISC_FEATURE (20)..(26): D-amino acid
CQTWRRVSPEECRKYKEEYNCVRCTE

SEQ ID NO: 13 - protein, 27 aa, Artificial Sequence
  Synthetic peptide
  MISC_FEATURE (20)..(20): D-amino acid
NDKCKELKKRYPNCEVRCDPPRYEVHC

SEQ ID NO: 14 - protein, 27 aa, Artificial Sequence
  Synthetic peptide
  MISC_FEATURE (1)..(19): D-amino acid
  MISC_FEATURE (21)..(27): D-amino acid
NDKCKELKKRYPNCEVRCDPPRYEVHC

SEQ ID NO: 15 - protein, 26 aa, Artificial Sequence
  Synthetic peptide
  MISC_FEATURE (6)..(7): D-amino acid
TCVECAPVKVCRPDPEEARREAEERC

SEQ ID NO: 16 - protein, 26 aa, Artificial Sequence
  Synthetic peptide
  MISC_FEATURE (1)..(5): D-amino acid
  MISC_FEATURE (8)..(26): D-amino acid
TCVECAPVKVCRPDPEEARREAEERC

SEQ ID NO: 17 - protein, 26 aa, Artificial Sequence
  Synthetic peptide
PDPNRCEEYKRKVPNEDEVRKYCKKF

SEQ ID NO: 18 - protein, 26 aa, Artificial Sequence
  Synthetic peptide
  MISC_FEATURE (1)..(26): D-amino acid
PDPNRCEEYKRKVPNEDEVRKYCKKF

SEQ ID NO: 19 - protein, 26 aa, Artificial Sequence
  Synthetic peptide
PTDEKCEELKKRATDPEKRKELCKRA

SEQ ID NO: 20 - protein, 26 aa, Artificial Sequence
  Synthetic peptide
PTDEKCEELKKRATDPEKRKELCKRA

SEQ ID NO: 21 - protein, 26 aa, Artificial Sequence
  Synthetic peptide
  MISC_FEATURE (1)..(26): D-amino acid
PTDEKCEELKKRATDPEKRKELCKRA

SEQ ID NO: 22 - protein, 32 aa, Artificial Sequence
  Synthetic peptide
CDPRQKKTWTERARKSASEEEKKTWKDQCSKG

SEQ ID NO: 23 - protein, 32 aa, Artificial Sequence
  Synthetic peptide
ASPEYKKECEKRERDGDDPREISKCKTNAKRG

SEQ ID NO: 24 - protein, 32 aa, Artificial Sequence
  Synthetic peptide
QTEECKKKADEWKKKAEDPREHKKADELKKKC

SEQ ID NO: 25 - protein, 32 aa, Artificial Sequence
  Synthetic peptide
QSEECKKKADEWAKKAEDPREHETAKELKKKC

SEQ ID NO: 26 - protein, 32 aa, Artificial Sequence
  Synthetic peptide
QDPDCQSKAREKLKKAQNPEQKKDAKRIEKEC

SEQ ID NO: 27 - protein, 32 aa, Artificial Sequence
  Synthetic peptide
CSEEDEKKAKKLDKDGDDPRKAESLKRKCKKG

SEQ ID NO: 28 - protein, 32 aa, Artificial Sequence
  Synthetic peptide
SDPEEQKDLKRLIKECTDPDCRKDLKRKIKET

SEQ ID NO: 29 - protein, 32 aa, Artificial Sequence
  Synthetic peptide
QDPTCQKQADEWAKKAQDPNQKKHYKKLKETC

SEQ ID NO: 30 - protein, 32 aa, Artificial Sequence
  Synthetic peptide
ASEEWKDRCDKWKKSGADPSIQKECDEKIKKG

SEQ ID NO: 31 - protein, 32 aa, Artificial Sequence
  Synthetic peptide
ASPEECSKYRKLIKDGASEEEQKKFKKYCKDG

SEQ ID NO: 32 - protein, 32 aa, Artificial Sequence
  Synthetic peptide
PNPEKCSKAEELKRKYPDPTVQKKADELCKKD

SEQ ID NO: 33 - protein, 32 aa, Artificial Sequence
  Synthetic peptide
SDPDQHKKADELKKKCQTPECKTKADEWKKKA

SEQ ID NO: 34 - protein, 32 aa, Artificial Sequence
  Synthetic peptide
QSEECKKKADEWAKKAEDPTEHEQAKELKKKC

SEQ ID NO: 35 - protein, 32 aa, Artificial Sequence
  Synthetic peptide
ASPEICKKAEEAEKKNDDPRKIKELQEKCKKG

SEQ ID NO: 36 - protein, 32 aa, Artificial Sequence
  Synthetic peptide
CSEEDKKKAKTWKDQGADPTIQKKADDKCSKG

SEQ ID NO: 37 - protein, 32 aa, Artificial Sequence
  Synthetic peptide
CSDEQRKTAEELEKKGDDPTKIKKAKDTCSKG

SEQ ID NO: 38 - protein, 32 aa, Artificial Sequence
  Synthetic peptide
CSEEDKKRLEEARKKGADPTEIKKLTEKCQKG

SEQ ID NO: 39 - protein, 32 aa, Artificial Sequence
  Synthetic peptide
SDKECRDRLKKLIKDIPDPEARKELEKRAREC

SEQ ID NO: 40 - protein, 32 aa, Artificial Sequence
  Synthetic peptide
QDPRAKETAKEWKKKCQTEECQKRADKYAKDH

SEQ ID NO: 41 - protein, 32 aa, Artificial Sequence
  Synthetic peptide
ASEEICKKAEEAKKKGDDPKKIKTLDELCKKG

SEQ ID NO: 42 - protein, 32 aa, Artificial Sequence
  Synthetic peptide
DDPTVCKQAEEAKKKGDDPRKIKTLDTRCKQG

SEQ ID NO: 43 - protein, 32 aa, Artificial Sequence
  Synthetic peptide
ADPEQCKTWEKQAKEGADPSQQKDWKRKCKEG

SEQ ID NO: 44 - protein, 32 aa, Artificial Sequence
  Synthetic peptide
SSEEVCKSAEEAKKKGDDEKKAKDLDKECKDG

SEQ ID NO: 45 - protein, 32 aa, Artificial Sequence
  Synthetic peptide
ASPEECSKYRKLIKDGASEEEQKKYKKACKDG

SEQ ID NO: 46 - protein, 32 aa, Artificial Sequence
  Synthetic peptide
ADPTQCKRWKEEAKKGADPSQQETWEKQCKSG

SEQ ID NO: 47 - protein, 32 aa, Artificial Sequence
  Synthetic peptide
KDPKEQKKAKEQYKKCQTKECKDKAKERLDKA

SEQ ID NO: 48 - protein, 32 aa, Artificial Sequence
  Synthetic peptide
QSEECKKKADEWKKKAEDPEERKKAEELKQKC

SEQ ID NO: 49 - protein, 32 aa, Artificial Sequence
  Synthetic peptide
SDPECQKTLDTLIKQIPDPETQKDLKKKKKEC

SEQ ID NO: 50 - protein, 32 aa, Artificial Sequence
  Synthetic peptide
SDPSDCKTAEELKRKGDDPEKIKHYETLCKRG

SEQ ID NO: 51 - protein, 32 aa, Artificial Sequence
  Synthetic peptide
GSEEDCKTAEKLKKDGADPREIKTADEKCKKG

SEQ ID NO: 52 - protein, 32 aa, Artificial Sequence
  Synthetic peptide
QSEECKKKADTWKKQAQNPEERKKYDELKKKC

SEQ ID NO: 53 - protein, 32 aa, Artificial Sequence
  Synthetic peptide
DDPSVCKSAEKAKKKGDNPEKIKTLETRCKQG

SEQ ID NO: 54 - protein, 32 aa, Artificial Sequence
  Synthetic peptide
ASEEECDTARQLKEKGDDPTKIKHYDRRCKEG

SEQ ID NO: 55 - protein, 32 aa, Artificial Sequence
  Synthetic peptide
ASEEYKKTCEKKKKDGASEEEKKTCDENIKKG

SEQ ID NO: 56 - protein, 32 aa, Artificial Sequence
  Synthetic peptide
CSEEDKKKLEEARRKGDDPTNIKRLEDKCKKG

SEQ ID NO: 57 - protein, 32 aa, Artificial Sequence
  Synthetic peptide
ADPSVCKKAEEAKKKGDDPRRIKTWDELCKKG

SEQ ID NO: 58 - protein, 32 aa, Artificial Sequence
  Synthetic peptide
ASPEICTKAEEAEKKGDDPRKIKELQDKCKKG

SEQ ID NO: 59 - protein, 32 aa, Artificial Sequence
  Synthetic peptide
CSEEDKKTAETLKRQGADPTEQKKMDDKCSKG

SEQ ID NO: 60 - protein, 32 aa, Artificial Sequence
  Synthetic peptide
SDPETQKKLEEKAQKCSDPECRKTLKKLIKDT

SEQ ID NO: 61 - protein, 32 aa, Artificial Sequence
  Synthetic peptide
SDEDCQKTLDKLKKDVPDPNQQKEYDERKKKC

SEQ ID NO: 62 - protein, 32 aa, Artificial Sequence; identical to SEQ ID NO: 22
  Synthetic peptide
  MISC_FEATURE (1)..(32): D-amino acid

SEQ ID NO: 63 - protein, 32 aa, Artificial Sequence; identical to SEQ ID NO: 23
  Synthetic peptide
  MISC_FEATURE (1)..(32): D-amino acid

SEQ ID NO: 64 - protein, 32 aa, Artificial Sequence; identical to SEQ ID NO: 24
  Synthetic peptide
  MISC_FEATURE (1)..(32): D-amino acid

SEQ ID NO: 65 - protein, 32 aa, Artificial Sequence; identical to SEQ ID NO: 25
  Synthetic peptide
  MISC_FEATURE (1)..(32): D-amino acid

SEQ ID NO: 66 - protein, 32 aa, Artificial Sequence; identical to SEQ ID NO: 26
  Synthetic peptide
  MISC_FEATURE (1)..(32): D-amino acid

SEQ ID NO: 67 - protein, 32 aa, Artificial Sequence; identical to SEQ ID NO: 27
  Synthetic peptide
  MISC_FEATURE (1)..(32): D-amino acid

SEQ ID NO: 68 - protein, 32 aa, Artificial Sequence; identical to SEQ ID NO: 28
  Synthetic peptide
  MISC_FEATURE (1)..(32): D-amino acid

SEQ ID NO: 69 - protein, 32 aa, Artificial Sequence; identical to SEQ ID NO: 29
  Synthetic peptide
  MISC_FEATURE (1)..(32): D-amino acid

SEQ ID NO: 70 - protein, 32 aa, Artificial Sequence; identical to SEQ ID NO: 30
  Synthetic peptide
  MISC_FEATURE (1)..(32): D-amino acid

SEQ ID NO: 71 - protein, 32 aa, Artificial Sequence; identical to SEQ ID NO: 31
  Synthetic peptide
  MISC_FEATURE (1)..(32): D-amino acid

SEQ ID NO: 72 - protein, 32 aa, Artificial Sequence; identical to SEQ ID NO: 32
  Synthetic peptide
  MISC_FEATURE (1)..(32): D-amino acid

SEQ ID NO: 73 - protein, 32 aa, Artificial Sequence; identical to SEQ ID NO: 33
  Synthetic peptide
  MISC_FEATURE (1)..(32): D-amino acid

SEQ ID NO: 74 - protein, 32 aa, Artificial Sequence; identical to SEQ ID NO: 34
  Synthetic peptide
  MISC_FEATURE (1)..(32): D-amino acid

SEQ ID NO: 75 - protein, 32 aa, Artificial Sequence; identical to SEQ ID NO: 35
  Synthetic peptide
  MISC_FEATURE (1)..(32): D-amino acid

SEQ ID NO: 76 - protein, 32 aa, Artificial Sequence; identical to SEQ ID NO: 36
  Synthetic peptide
  MISC_FEATURE (1)..(32): D-amino acid

SEQ ID NO: 77 - protein, 32 aa, Artificial Sequence; identical to SEQ ID NO: 37
  Synthetic peptide
  MISC_FEATURE (1)..(32): D-amino acid

SEQ ID NO: 78 - protein, 32 aa, Artificial Sequence; identical to SEQ ID NO: 38
  Synthetic peptide
  MISC_FEATURE (1)..(32): D-amino acid

SEQ ID NO: 79 - protein, 32 aa, Artificial Sequence; identical to SEQ ID NO: 39
  Synthetic peptide
  MISC_FEATURE (1)..(32): D-amino acid

SEQ ID NO: 80 - protein, 32 aa, Artificial Sequence; identical to SEQ ID NO: 40
  Synthetic peptide
  MISC_FEATURE (1)..(32): D-amino acid

SEQ ID NO: 81 - protein, 32 aa, Artificial Sequence; identical to SEQ ID NO: 41
  Synthetic peptide
  MISC_FEATURE (1)..(32): D-amino acid

SEQ ID NO: 82 - protein, 32 aa, Artificial Sequence; identical to SEQ ID NO: 42
  Synthetic peptide
  MISC_FEATURE (1)..(32): D-amino acid

SEQ ID NO: 83 - protein, 32 aa, Artificial Sequence; identical to SEQ ID NO: 43
  Synthetic peptide
  MISC_FEATURE (1)..(32): D-amino acid

SEQ ID NO: 84 - protein, 32 aa, Artificial Sequence; identical to SEQ ID NO: 44
  Synthetic peptide
  MISC_FEATURE (1)..(32): D-amino acid

SEQ ID NO: 85 - protein, 32 aa, Artificial Sequence; identical to SEQ ID NO: 45
  Synthetic peptide
  MISC_FEATURE (1)..(32): D-amino acid

SEQ ID NO: 86 - protein, 32 aa, Artificial Sequence; identical to SEQ ID NO: 46
  Synthetic peptide
  MISC_FEATURE (1)..(32): D-amino acid

SEQ ID NO: 87 - protein, 32 aa, Artificial Sequence; identical to SEQ ID NO: 47
  Synthetic peptide
  MISC_FEATURE (1)..(32): D-amino acid

SEQ ID NO: 88 - protein, 32 aa, Artificial Sequence; identical to SEQ ID NO: 48
  Synthetic peptide
  MISC_FEATURE (1)..(32): D-amino acid

SEQ ID NO: 89 - protein, 32 aa, Artificial Sequence; identical to SEQ ID NO: 49
  Synthetic peptide
  MISC_FEATURE (1)..(32): D-amino acid

SEQ ID NO: 90 - protein, 32 aa, Artificial Sequence; identical to SEQ ID NO: 50
  Synthetic peptide
  MISC_FEATURE (1)..(32): D-amino acid

SEQ ID NO: 91 - protein, 32 aa, Artificial Sequence; identical to SEQ ID NO: 51
  Synthetic peptide
  MISC_FEATURE (1)..(32): D-amino acid

SEQ ID NO: 92 - protein, 32 aa, Artificial Sequence; identical to SEQ ID NO: 52
  Synthetic peptide
  MISC_FEATURE (1)..(32): D-amino acid

SEQ ID NO: 93 - protein, 32 aa, Artificial Sequence; identical to SEQ ID NO: 53
  Synthetic peptide
  MISC_FEATURE (1)..(32): D-amino acid

SEQ ID NO: 94 - protein, 32 aa, Artificial Sequence; identical to SEQ ID NO: 54
  Synthetic peptide
  MISC_FEATURE (1)..(32): D-amino acid

SEQ ID NO: 95 - protein, 32 aa, Artificial Sequence; identical to SEQ ID NO: 55
  Synthetic peptide
  MISC_FEATURE (1)..(32): D-amino acid

SEQ ID NO: 96 - protein, 32 aa, Artificial Sequence; identical to SEQ ID NO: 56
  Synthetic peptide
  MISC_FEATURE (1)..(32): D-amino acid

SEQ ID NO: 97 - protein, 32 aa, Artificial Sequence; identical to SEQ ID NO: 57
  Synthetic peptide
  MISC_FEATURE (1)..(32): D-amino acid

SEQ ID NO: 98 - protein, 32 aa, Artificial Sequence; identical to SEQ ID NO: 58
  Synthetic peptide
  MISC_FEATURE (1)..(32): D-amino acid

SEQ ID NO: 99 - protein, 32 aa, Artificial Sequence; identical to SEQ ID NO: 59
  Synthetic peptide
  MISC_FEATURE (1)..(32): D-amino acid

SEQ ID NO: 100 - protein, 32 aa, Artificial Sequence; identical to SEQ ID NO: 60
  Synthetic peptide
  MISC_FEATURE (1)..(32): D-amino acid

SEQ ID NO: 101 - protein, 32 aa, Artificial Sequence; identical to SEQ ID NO: 61
  Synthetic peptide
  MISC_FEATURE (1)..(32): D-amino acid

SEQ ID NO: 102 - protein, 26 aa, Artificial Sequence
  Synthetic peptide
YTVCCNGICYTNDNKDEAEKVKKKIC

SEQ ID NO: 103 - protein, 26 aa, Artificial Sequence
  Synthetic peptide
TCVECNGVKVCRPDPEEARRLAEEKC

SEQ ID NO: 104 - protein, 26 aa, Artificial Sequence
  Synthetic peptide
CRVCENNFCVDASSCEEAQRILEKYK

SEQ ID NO: 105 - protein, 26 aa, Artificial Sequence
  Synthetic peptide
TRCCINGYCVESDSTKEVEDKCKKYA

SEQ ID NO: 106 - protein, 26 aa, Artificial Sequence
  Synthetic peptide
TTVCINGFCCTAPTPEEAKRCAKELS

SEQ ID NO: 107 - protein, 26 aa, Artificial Sequence
  Synthetic peptide
VTVCINGYCCTAPTPDEAEECARRLS

SEQ ID NO: 108 - protein, 26 aa, Artificial Sequence
  Synthetic peptide
ACVTYCHVTVCTKDPEEAKRKAKEIC

SEQ ID NO: 109 - protein, 26 aa, Artificial Sequence
  Synthetic peptide
CEVTYCNITVRAESCEKAEKIARKLC

SEQ ID NO: 110 - protein, 26 aa, Artificial Sequence
  Synthetic peptide
LCICVNGECICIPNPDEARKAEKKMR

SEQ ID NO: 111 - protein, 26 aa, Artificial Sequence
  Synthetic peptide
ACVTVCGYTVCRPDPEEARRIAEELC

SEQ ID NO: 112 - protein, 26 aa, Artificial Sequence
  Synthetic peptide
VKVCICGYCYTASTDEEAKQAKKEMC

SEQ ID NO: 113 - protein, 26 aa, Artificial Sequence
  Synthetic peptide
CCLTFGGRTFCADDCEEAKKLAKKAG

SEQ ID NO: 114 - protein, 26 aa, Artificial Sequence
  Synthetic peptide
YCITCGNETYCSDDPEDAKRLCKEAL

SEQ ID NO: 115 - protein, 26 aa, Artificial Sequence
  Synthetic peptide
YCFTLKGCTVCAPNPEDAKTELKKCA

SEQ ID NO: 116 - protein, 26 aa, Artificial Sequence
  Synthetic peptide
ACVCVNGVCVCASSPQEAEEIARKIR

SEQ ID NO: 117 - protein, 26 aa, Artificial Sequence
  Synthetic peptide
VTERYGDCEIHCPTQDCADQYKEECK

SEQ ID NO: 118 - protein, 26 aa, Artificial Sequence
  Synthetic peptide
CEVQIDDCRVPACTEDEAKELCKKGE

SEQ ID NO: 119 - protein, 26 aa, Artificial Sequence
  Synthetic peptide
CEVTLNGCTYRASSCEEAKRYLEKYC

SEQ ID NO: 120 - protein, 26 aa, Artificial Sequence
  Synthetic peptide
STVCCNGYCEEAHDEDEEREIRERCK

SEQ ID NO: 121 - protein, 26 aa, Artificial Sequence
  Synthetic peptide
YCITCNNQTFCAPDPEKAKELCKRAL

SEQ ID NO: 122 - protein, 26 aa, Artificial Sequence
  Synthetic peptide
TELRRGDLRCECSTDEECKRLSKEIC

SEQ ID NO: 123 - protein, 26 aa, Artificial Sequence
  Synthetic peptide
CKVKCGPVEYQATSQDECNEWRKKYC

SEQ ID NO: 124 - protein, 27 aa, Artificial Sequence
  Synthetic peptide
PPECEKYKKKYPNCQVTTDNGQCTFRC

SEQ ID NO: 125 - protein, 27 aa, Artificial Sequence
  Synthetic peptide
SDECEKLKKKYPNCKVEDHNGECRVKC

SEQ ID NO: 126 - protein, 27 aa, Artificial Sequence
  Synthetic peptide
EPQCEELKRRYPNCTVTKDGNTCKVDC

SEQ ID NO: 127 - protein, 27 aa, Artificial Sequence
  Synthetic peptide
NPECEKYKKKYPNCDVKEKNGQCTFEC

SEQ ID NO: 128 - protein, 27 aa, Artificial Sequence
  Synthetic peptide
PPQCEEYKKKYPNCEVRDHNGECRVHC

SEQ ID NO: 129 - protein, 27 aa, Artificial Sequence
  Synthetic peptide
SEDCKELQKKFPECQVEEHNGDCQVRC

SEQ ID NO: 130 - protein, 27 aa, Artificial Sequence
  Synthetic peptide
YEKQKELQKKFPDCEVRCKDGQCQVHC

SEQ ID NO: 131 - protein, 27 aa, Artificial Sequence
  Synthetic peptide
TERCKEYKKRYPNCEVRSHGNTCKVQC

SEQ ID NO: 132 - protein, 27 aa, Artificial Sequence
  Synthetic peptide
SDKCKELKKRYPNCEVRCDGNRYEVHC

SEQ ID NO: 133 - protein, 27 aa, Artificial Sequence
  Synthetic peptide
PPECEKLKKKYPNCDVTCDNGDSQIQC

SEQ ID NO: 134 - protein, 27 aa, Artificial Sequence
  Synthetic peptide
SDECKEYKDKYPNCKVTQKNGQCHVQC

SEQ ID NO: 135 - protein, 27 aa, Artificial Sequence
  Synthetic peptide
TPECEKLKKKYPNCDVSEDNGDCQVRC

SEQ ID NO: 136 - protein, 27 aa, Artificial Sequence
  Synthetic peptide
SDEQRQLEEKRPDCEVRCRGTTCELKC

SEQ ID NO: 137 - protein, 27 aa, Artificial Sequence
  Synthetic peptide
YECERQLKEKYPDCEVRVQDTECRWRC

SEQ ID NO: 138 - protein, 27 aa, Artificial Sequence
  Synthetic peptide
CPIAEELKKRFPNCKVECHGDEYRVHC

SEQ ID NO: 139 - protein, 27 aa, Artificial Sequence
  Synthetic peptide
YEREKELQKRFPNCEVRCRSNQCQVNC

SEQ ID NO: 140 - protein, 27 aa, Artificial Sequence
  Synthetic peptide
SDECEEYKRKYPNCTVEQKGNTCEYRC

SEQ ID NO: 141 - protein, 27 aa, Artificial Sequence
  Synthetic peptide
NPRCEEYKKRYPNCEVRDDNGRCEYRC

SEQ ID NO: 142 - protein, 27 aa, Artificial Sequence
  Synthetic peptide
QPECEKLKRKYPNCEVTQDGTQCKVRC

SEQ ID NO: 143 - protein, 27 aa, Artificial Sequence
  Synthetic peptide
TERCKEYKKRYPTCRVEDDNGDCRVHC

SEQ ID NO: 144 - protein, 27 aa, Artificial Sequence
  Synthetic peptide
SDTCEELKRRYKNCEVRCRGTEYEVRC

SEQ ID NO: 145 - protein, 27 aa, Artificial Sequence
  Synthetic peptide
SDRCEEYKRRYPNCEVRDENGNCKVRC

SEQ ID NO: 146 - protein, 27 aa, Artificial Sequence
  Synthetic peptide
TPQCEEYKKRYPNCEVEDDNGDCQVRC

SEQ ID NO: 147 - protein, 27 aa, Artificial Sequence
  Synthetic peptide
SEKCKELKKKYPNCEVREDNGRCEVHC

SEQ ID NO: 148 - protein, 27 aa, Artificial Sequence
  Synthetic peptide
NPECEKLKKKYPNCNVECDNGDTRIEC

SEQ ID NO: 149 - protein, 27 aa, Artificial Sequence
  Synthetic peptide
GEKCKEYKKKYPNCRVEERNGDCQVTC

SEQ ID NO: 150 - protein, 27 aa, Artificial Sequence
  Synthetic peptide
SQECEDYKEKYRNCQISEDNGQCTFQC

SEQ ID NO: 151 - protein, 27 aa, Artificial Sequence
  Synthetic peptide
DEDCEELKRRYKSCDVTKSGGQCKVDC

SEQ ID NO: 152 - protein, 27 aa, Artificial Sequence
  Synthetic peptide
NPRCEEYKRRWPNCEVREHNGQCTYRC

SEQ ID NO: 153 - protein, 26 aa, Artificial Sequence
  Synthetic peptide
  MISC_FEATURE (1)..(6): D-amino acid
  MISC_FEATURE (8)..(26): D-amino acid
YTVCCNGICYTNDNKDEAEKVKKKIC

SEQ ID NO: 154 - protein, 26 aa, Artificial Sequence
  Synthetic peptide
  MISC_FEATURE (1)..(6): D-amino acid
  MISC_FEATURE (8)..(26): D-amino acid
TCVECNGVKVCRPDPEEARRLAEEKC

SEQ ID NO: 155 - protein, 26 aa, Artificial Sequence
  Synthetic peptide
  MISC_FEATURE (1)..(26): D-amino acid
CRVCENNFCVDASSCEEAQRILEKYK

SEQ ID NO: 156 - protein, 26 aa, Artificial Sequence
  Synthetic peptide
  MISC_FEATURE (1)..(6): D-amino acid
  MISC_FEATURE (8)..(26): D-amino acid
TRCCINGYCVESDSTKEVEDKCKKYA

SEQ ID NO: 157 - protein, 26 aa, Artificial Sequence
  Synthetic peptide
  MISC_FEATURE (1)..(6): D-amino acid
  MISC_FEATURE (8)..(26): D-amino acid
TTVCINGFCCTAPTPEEAKRCAKELS

SEQ ID NO: 158 - protein, 26 aa, Artificial Sequence
  Synthetic peptide
  MISC_FEATURE (1)..(6): D-amino acid
  MISC_FEATURE (8)..(26): D-amino acid
VTVCINGYCCTAPTPDEAEECARRLS

SEQ ID NO: 159 - protein, 26 aa, Artificial Sequence
  Synthetic peptide
  MISC_FEATURE (1)..(26): D-amino acid
ACVTYCHVTVCTKDPEEAKRKAKEIC

SEQ ID NO: 160 - protein, 26 aa, Artificial Sequence
  Synthetic peptide
  MISC_FEATURE (1)..(26): D-amino acid
CEVTYCNITVRAESCEKAEKIARKLC

SEQ ID NO: 161 - protein, 26 aa, Artificial Sequence
  Synthetic peptide
  MISC_FEATURE (1)..(6): D-amino acid
  MISC_FEATURE (8)..(26): D-amino acid
LCICVNGECICIPNPDEARKAEKKMR

SEQ ID NO: 162 - protein, 26 aa, Artificial Sequence
  Synthetic peptide
  MISC_FEATURE (1)..(6): D-amino acid
  MISC_FEATURE (8)..(26): D-amino acid
ACVTVCGYTVCRPDPEEARRIAEELC

SEQ ID NO: 163 - protein, 26 aa, Artificial Sequence
  Synthetic peptide
  MISC_FEATURE (1)..(6): D-amino acid
  MISC_FEATURE (8)..(26): D-amino acid
VKVCICGYCYTASTDEEAKQAKKEMC

SEQ ID NO: 164 - protein, 26 aa, Artificial Sequence
  Synthetic peptide
  MISC_FEATURE (1)..(5): D-amino acid
  MISC_FEATURE (8)..(25): D-amino acid
CCLTFGGRTFCADDCEEAKKLAKKAG

SEQ ID NO: 165 - protein, 26 aa, Artificial Sequence
  Synthetic peptide
  MISC_FEATURE (1)..(5): D-amino acid
  MISC_FEATURE (7)..(26): D-amino acid
YCITCGNETYCSDDPEDAKRLCKEAL

SEQ ID NO: 166 - protein, 26 aa, Artificial Sequence
  Synthetic peptide
  MISC_FEATURE (1)..(6): D-amino acid
  MISC_FEATURE (8)..(26): D-amino acid
YCFTLKGCTVCAPNPEDAKTELKKCA

SEQ ID NO: 167 - protein, 26 aa, Artificial Sequence
  Synthetic peptide
  MISC_FEATURE (1)..(6): D-amino acid
ACVCVNGVCVCASSPQEAEEIARKIR

SEQ ID NO: 168 - protein, 26 aa, Artificial Sequence
  Synthetic peptide
  MISC_FEATURE (1)..(5): D-amino acid
  MISC_FEATURE (7)..(26): D-amino acid
VTERYGDCEIHCPTQDCADQYKEECK

SEQ ID NO: 169 - protein, 26 aa, Artificial Sequence
  Synthetic peptide
  MISC_FEATURE (1)..(24): D-amino acid
  MISC_FEATURE (26)..(26): D-amino acid
CEVQIDDCRVPACTEDEAKELCKKGE

SEQ ID NO: 170 - protein, 26 aa, Artificial Sequence
  Synthetic peptide
  MISC_FEATURE (1)..(6): D-amino acid
  MISC_FEATURE (8)..(26): D-amino acid
CEVTLNGCTYRASSCEEAKRYLEKYC

SEQ ID NO: 171 - protein, 26 aa, Artificial Sequence
  Synthetic peptide
  MISC_FEATURE (1)..(6): D-amino acid
  MISC_FEATURE (8)..(26): D-amino acid
STVCCNGYCEEAHDEDEEREIRERCK

SEQ ID NO: 172 - protein, 26 aa, Artificial Sequence
  Synthetic peptide
  MISC_FEATURE (1)..(26): D-amino acid
YCITCNNQTFCAPDPEKAKELCKRAL

SEQ ID NO: 173 - protein, 26 aa, Artificial Sequence
  Synthetic peptide
  MISC_FEATURE (1)..(5): D-amino acid
  MISC_FEATURE (7)..(27): D-amino acid
TELRRGDLRCECSTDEECKRLSKEIC

SEQ ID NO: 174 - protein, 26 aa, Artificial Sequence
  Synthetic peptide
  MISC_FEATURE (1)..(5): D-amino acid
  MISC_FEATURE (7)..(26): D-amino acid
CKVKCGPVEYQATSQDECNEWRKKYC

SEQ ID NO: 175 - protein, 27 aa, Artificial Sequence
  Synthetic peptide
  MISC_FEATURE (1)..(20): D-amino acid
  MISC_FEATURE (22)..(27): D-amino acid
PPECEKYKKKYPNCQVTTDNGQCTFRC

SEQ ID NO: 176 - protein, 27 aa, Artificial Sequence
  Synthetic peptide
  MISC_FEATURE (1)..(20): D-amino acid
  MISC_FEATURE (22)..(27): D-amino acid
SDECEKLKKKYPNCKVEDHNGECRVKC

SEQ ID NO: 177 - protein, 27 aa, Artificial Sequence
  Synthetic peptide
  MISC_FEATURE (1)..(19): D-amino acid
  MISC_FEATURE (21)..(27): D-amino acid
EPQCEELKRRYPNCTVTKDGNTCKVDC

SEQ ID NO: 178 - protein, 27 aa, Artificial Sequence
  Synthetic peptide
  MISC_FEATURE (1)..(20): D-amino acid
  MISC_FEATURE (22)..(27): D-amino acid
NPECEKYKKKYPNCDVKEKNGQCTFEC

SEQ ID NO: 179 - protein, 27 aa, Artificial Sequence
  Synthetic peptide
  MISC_FEATURE (1)..(20): D-amino acid
  MISC_FEATURE (22)..(27): D-amino acid
PPQCEEYKKKYPNCEVRDHNGECRVHC

SEQ ID NO: 180 - protein, 27 aa, Artificial Sequence
  Synthetic peptide
  MISC_FEATURE (1)..(20): D-amino acid
  MISC_FEATURE (22)..(27): D-amino acid
SEDCKELQKKFPECQVEEHNGDCQVRC

SEQ ID NO: 181 - protein, 27 aa, Artificial Sequence
  Synthetic peptide
  MISC_FEATURE (1)..(20): D-amino acid
  MISC_FEATURE (22)..(27): D-amino acid
YEKQKELQKKFPDCEVRCKDGQCQVHC

SEQ ID NO: 182 - protein, 27 aa, Artificial Sequence
  Synthetic peptide
  MISC_FEATURE (1)..(19): D-amino acid
  MISC_FEATURE (21)..(27): D-amino acid
TERCKEYKKRYPNCEVRSHGNTCKVQC

SEQ ID NO: 183 - protein, 27 aa, Artificial Sequence
  Synthetic peptide
  MISC_FEATURE (1)..(19): D-amino acid
  MISC_FEATURE (21)..(27): D-amino acid
SDKCKELKKRYPNCEVRCDGNRYEVHC

SEQ ID NO: 184 - protein, 27 aa, Artificial Sequence
  Synthetic peptide
  MISC_FEATURE (1)..(20): D-amino acid
  MISC_FEATURE (22)..(27): D-amino acid
PPECEKLKKKYPNCDVTCDNGDSQIQC

SEQ ID NO: 185 - protein, 27 aa, Artificial Sequence
  Synthetic peptide
SDECKEYKDKYPNCKVTQKNGQCHVQC

SEQ ID NO: 186 - protein, 27 aa, Artificial Sequence
  Synthetic peptide
  MISC_FEATURE (1)..(20): D-amino acid
  MISC_FEATURE (22)..(27): D-amino acid
TPECEKLKKKYPNCDVSEDNGDCQVRC

SEQ ID NO: 187 - protein, 27 aa, Artificial Sequence
  Synthetic peptide
  MISC_FEATURE (1)..(19): D-amino acid
  MISC_FEATURE (21)..(27): D-amino acid
SDEQRQLEEKRPDCEVRCRGTTCELKC

SEQ ID NO: 188 - protein, 27 aa, Artificial Sequence
  Synthetic peptide
  MISC_FEATURE (1)..(27): D-amino acid
YECERQLKEKYPDCEVRVQDTECRWRC

SEQ ID NO: 189 - protein, 27 aa, Artificial Sequence
  Synthetic peptide
  MISC_FEATURE (1)..(19): D-amino acid
  MISC_FEATURE (21)..(27): D-amino acid
CPIAEELKKRFPNCKVECHGDEYRVHC

SEQ ID NO: 190 - protein, 27 aa, Artificial Sequence
  Synthetic peptide
  MISC_FEATURE (1)..(27): D-amino acid
YEREKELQKRFPNCEVRCRSNQCQVNC

SEQ ID NO: 191 - protein, 27 aa, Artificial Sequence
  Synthetic peptide
  MISC_FEATURE (1)..(19): D-amino acid
  MISC_FEATURE (21)..(27): D-amino acid
SDECEEYKRKYPNCTVEQKGNTCEYRC

SEQ ID NO: 192 - protein, 27 aa, Artificial Sequence
  Synthetic peptide
  MISC_FEATURE (1)..(20): D-amino acid
  MISC_FEATURE (22)..(27): D-amino acid
NPRCEEYKKRYPNCEVRDDNGRCEYRC

SEQ ID NO: 193 - protein, 27 aa, Artificial Sequence
  Synthetic peptide
  MISC_FEATURE (1)..(19): D-amino acid
  MISC_FEATURE (21)..(27): D-amino acid
QPECEKLKRKYPNCEVTQDGTQCKVRC

SEQ ID NO: 194 - protein, 27 aa, Artificial Sequence
  Synthetic peptide
  MISC_FEATURE (1)..(20): D-amino acid
  MISC_FEATURE (22)..(27): D-amino acid
TERCKEYKKRYPTCRVEDDNGDCRVHC

SEQ ID NO: 195 - protein, 27 aa, Artificial Sequence
  Synthetic peptide
  MISC_FEATURE (1)..(19): D-amino acid
  MISC_FEATURE (21)..(27): D-amino acid
SDTCEELKRRYKNCEVRCRGTEYEVRC

SEQ ID NO: 196 - protein, 27 aa, Artificial Sequence
  Synthetic peptide
  MISC_FEATURE (1)..(20): D-amino acid
  MISC_FEATURE (22)..(27): D-amino acid
SDRCEEYKRRYPNCEVRDENGNCKVRC

SEQ ID NO: 197 - protein, 27 aa, Artificial Sequence
  Synthetic peptide
  MISC_FEATURE (1)..(20): D-amino acid
  MISC_FEATURE (22)..(27): D-amino acid
TPQCEEYKKRYPNCEVEDDNGDCQVRC

SEQ ID NO: 198 - protein, 27 aa, Artificial Sequence
  Synthetic peptide
  MISC_FEATURE (1)..(20): D-amino acid
  MISC_FEATURE (22)..(27): D-amino acid
SEKCKELKKKYPNCEVREDNGRCEVHC

SEQ ID NO: 199 - protein, 27 aa, Artificial Sequence
  Synthetic peptide
  MISC_FEATURE (1)..(20): D-amino acid
  MISC_FEATURE (22)..(27): D-amino acid
NPECEKLKKKYPNCNVECDNGDTRIEC

SEQ ID NO: 200 - protein, 27 aa, Artificial Sequence
  Synthetic peptide
  MISC_FEATURE (2)..(20): D-amino acid
  MISC_FEATURE (22)..(27): D-amino acid
GEKCKEYKKKYPNCRVEERNGDCQVTC

SEQ ID NO: 201 - protein, 27 aa, Artificial Sequence
  Synthetic peptide
  MISC_FEATURE (1)..(20): D-amino acid
  MISC_FEATURE (22)..(27): D-amino acid
SQECEDYKEKYRNCQISEDNGQCTFQC

SEQ ID NO: 202 - protein, 27 aa, Artificial Sequence
  Synthetic peptide
  MISC_FEATURE (1)..(19): D-amino acid
  MISC_FEATURE (22)..(27): D-amino acid
DEDCEELKRRYKSCDVTKSGGQCKVDC

SEQ ID NO: 203 - protein, 27 aa, Artificial Sequence
  Synthetic peptide
  MISC_FEATURE (1)..(20): D-amino acid
  MISC_FEATURE (22)..(27): D-amino acid
NPRCEEYKRRWPNCEVREHNGQCTYRC

SEQ ID NO: 204 - protein, 46 aa, Artificial Sequence
  Synthetic peptide
CRFRAECQGNNVHVRGDGCKKEEIEKAWKKAEEWCKNGMQSSEREE

SEQ ID NO: 205 - protein, 39 aa, Artificial Sequence
  Synthetic peptide
CCKQQNENCYFAERTNKTFCYQDSKEQAREDCEEECRRS

SEQ ID NO: 206 - protein, 36 aa, Artificial Sequence
  Synthetic peptide
CSDCETECYCFVSKGKQWHGTSEECKKYKEEAEREC

SEQ ID NO: 207 - protein, 40 aa, Artificial Sequence
  Synthetic peptide
SCEEEAKKEADKCRKNGCQYRVDSDNCEVECRNCNIRKQF

SEQ ID NO: 208 - protein, 41 aa, Artificial Sequence
  Synthetic peptide
DCFFVIGGQDDQQCHTHQEECRKECEEKAEEQNRQCFDHCT

SEQ ID NO: 209 - protein, 42 aa, Artificial Sequence
  Synthetic peptide
KCYVICGNHDDYEFDTTREEECRRECEKARQEQNHECNCHYS

SEQ ID NO: 210 - protein, 43 aa, Artificial Sequence
  Synthetic peptide
EQYHCHGNYVRYICEDGQDCEYHADCSDEEAEREAKEECERQC

SEQ ID NO: 211 - protein, 42 aa, Artificial Sequence
  Synthetic peptide
KPEEYCRKVKDECKKRGLTRCHVTAKYGCECEVRGDTYQLRC

SEQ ID NO: 212 - protein, 51 aa, Artificial Sequence
  Synthetic peptide
ECEKKAEECKRYAEEQNTSEECAERAEEYARRHCESSEEECREYAEECKKN

SEQ ID NO: 213 - protein, 43 aa, Artificial Sequence
  Synthetic peptide
PCEDLKERLKKLGMSEECRQRLEKMCKEGTSEDAERMARNCES

SEQ ID NO: 214 - protein, 38 aa, Artificial Sequence
  Synthetic peptide
TCQERVKEIKERCKKRGQEIRERPGDHEVQCGTERYRC

SEQ ID NO: 215 - protein, 40 aa, Artificial Sequence
  Synthetic peptide
TCETYHVKRPDCREAEEEARKLRQECKDRGQCCTVTWTCK

SEQ ID NO: 216 - protein, 50 aa, Artificial Sequence
  Synthetic peptide
PCQECERELEEAKRNNQCREERAEEIRREREEGQTSCEECKREAERCRQE

SEQ ID NO: 217 - protein, 44 aa, Artificial Sequence
  Synthetic peptide
SECSKEACKQAETGTCDQFDEWLKRQGCPPTEDLDECRKRCKEN

SEQ ID NO: 218 - protein, 37 aa, Artificial Sequence
  Synthetic peptide
CHITITCTHGTETRTETVKTTDPNECEKREKEIKNRC

SEQ ID NO: 219 - protein, 27 aa, Artificial Sequence
  Synthetic peptide
AQCEKDLKKVKKTGDPEKLDKIRKKCA

SEQ ID NO: 220 - protein, 43 aa, Artificial Sequence
  Synthetic peptide
PCWKELKKSAEKRGNEKCKKLAEECHRRNLSCDECEKLYRKCS

SEQ ID NO: 221 - protein, 26 aa, Artificial Sequence
  Synthetic peptide
CEKFKCNGQTYKYCDPNEAKKAKKKC

SEQ ID NO: 222 - protein, 32 aa, Artificial Sequence
  Synthetic peptide
NCQINGDTCQIGNEQCQNQEECKRLCEECEKS

SEQ ID NO: 223 - protein, 37 aa, Artificial Sequence
  Synthetic peptide
CVQRHPGKKVRCGNREEYQCTTDECVREMEEKCEKRC

SEQ ID NO: 224 - protein, 39 aa, Artificial Sequence
  Synthetic peptide
CVRCRHGNEERTYCCTSEECKREVKEKCDNDSTSRFHTG

SEQ ID NO: 225 - protein, 32 aa, Artificial Sequence
  Synthetic peptide
KTCEFTIPNCSEEEARRYSKKKGCDETRWQCG

SEQ ID NO: 226 - protein, 38 aa, Artificial Sequence
  Synthetic peptide
DCEIRSQCSHVRTDDPNECERICKECKKRGYEVHCDNR

SEQ ID NO: 227 - protein, 32 aa, Artificial Sequence
  Synthetic peptide
ADCDKKLKKVQEKSKKGLTETVRKLKEKVEKC

SEQ ID NO: 228 - protein, 37 aa, Artificial Sequence
  Synthetic peptide
QCVRFEFRPNDEEKKRKAEKACRELKKEGKCCEEKEG

SEQ ID NO: 229 - protein, 31 aa, Artificial Sequence
  Synthetic peptide
TCIKYTNPNCGRTVERCGQDPEKIKKEASKC

SEQ ID NO: 230 - protein, 40 aa, Artificial Sequence
  Synthetic peptide
CRIEVRGTEVRCCDGTRCERYEMTSKEEAKKMEKKCRKKC

SEQ ID NO: 231 - protein, 45 aa, Artificial Sequence
  Synthetic peptide
DREERRCRGGKEEECRREAEKRCKEHNGTCEVRKQGNEIRIEIRR

SEQ ID NO: 232 - protein, 42 aa, Artificial Sequence
  Synthetic peptide
CKEEMEKVCKEIGTEEKCKRIRKVAERGNCEEAQREAKRMKS

SEQ ID NO: 233 - protein, 40 aa, Artificial Sequence
  Synthetic peptide
CQEDIDGSHYRCFIRQTGSHCQCTTEECAKECDRQCEEEC

SEQ ID NO: 234 - protein, 45 aa, Artificial Sequence
  Synthetic peptide
NRDRRCYSSGRAEEIARRLAEEARRKGKTYEERKTGGTICVEIDE

SEQ ID NO: 235 - protein, 42 aa, Artificial Sequence
  Synthetic peptide
SDDKAEQCCKEIGNEEKCRRLKEVAKDGSEEEVDEMCRRMRS

SEQ ID NO: 236 - protein, 44 aa, Artificial Sequence
  Synthetic peptide
SSECEKKICKEWKKGTSEDELRKLCSSCTNNDKECDEAIKKCKK

SEQ ID NO: 237 - protein, 41 aa, Artificial Sequence
  Synthetic peptide
CRCHITSSCVRVEGDNGEEYRYCSSDEEDLRRFCKEMQKQC

SEQ ID NO: 238 - protein, 48 aa, Artificial Sequence
  Synthetic peptide
TSCEEEIKKLCKSGKRDPEEEKKVEKICRKCGVSEDQCEELKKKFRKC

SEQ ID NO: 239 - protein, 42 aa, Artificial Sequence
  Synthetic peptide
CTTFRFTSPCGNTEVRVTTCDPNEKKEAQKEAEKLKKKCKKS

SEQ ID NO: 240 - protein, 40 aa, Artificial Sequence
  Synthetic peptide
SEECAERLREECERRNIPYEVRKTSTCITVQCGTERYTCC

SEQ ID NO: 241 - protein, 50 aa, Artificial Sequence
  Synthetic peptide
KCEEAEREARECQENNQCREEELEKIEEKREKGETSCEEAKEEIERCCQS

SEQ ID NO: 242 - protein, 41 aa, Artificial Sequence
  Synthetic peptide
NPEDCARKVEEHCQRQGVRYTTHRQPTCIEVRCEKTTIRCC

SEQ ID NO: 243 - protein, 29 aa, Artificial Sequence
  Synthetic peptide
ADDIKKCEKKVRKDSNPDVKKKLKKCKKA

SEQ ID NO: 244 - protein, 51 aa, Artificial Sequence
  Synthetic peptide
KCWRKAKEECRKAQEGKTQEEECKEACRECKERGESSEEECKEAEKEARKE

SEQ ID NO: 245 - protein, 41 aa, Artificial Sequence
  Synthetic peptide
ECYFFIGGTDDQECQSEQEECRKKAEEKCREQNQQCVDDCK

SEQ ID NO: 246 - protein, 37 aa, Artificial Sequence
  Synthetic peptide
TCDCKDHETIFCNCPGNDDDQASTREECKKKCEERES

SEQ ID NO: 247 - protein, 45 aa, Artificial Sequence
  Synthetic peptide
EERRYKRCGQDEERVRRECKERGERQNCQYQIRKEGNCYVCEIRC

SEQ ID NO: 248 - protein, 44 aa, Artificial Sequence
  Synthetic peptide
CIVICDCETDDDDDQQNCREEEAREEARKREEECGEQFTCHVQT

SEQ ID NO: 249 - protein, 48 aa, Artificial Sequence
  Synthetic peptide
PVECRRTSKHVEVRCGNVQVRTSEDCQCSEKNNRVHIQCSKTREEYQC

SEQ ID NO: 250 - protein, 41 aa, Artificial Sequence
  Synthetic peptide
CCREEYQNHEWFVEHPEPRRFRCDNTRCEEAEERCDEECRK

SEQ ID NO: 251 - protein, 47 aa, Artificial Sequence
  Synthetic peptide
VCRIEWTTTSCRIDCGTEEYHVEPGKEICVGNFCVRVTNTTCTVQSN

SEQ ID NO: 252 - protein, 51 aa, Artificial Sequence
  Synthetic peptide
KECRIRHRGDKARVRVRDGGTSEEREVKCDGDDNKCKEAYQRICEEWERKR

SEQ ID NO: 253 - protein, 51 aa, Artificial Sequence
  Synthetic peptide
CQMREETRGNTIVMRVQGGRDSEEFRKKGGAREEEERKYRKKAEDKCKNNQ

SEQ ID NO: 254 - protein, 39 aa, Artificial Sequence
  Synthetic peptide
TCNVTCDNRDTQTFDDCEECKKKAKECKSEGRDVQIQCG

SEQ ID NO: 255 - protein, 45 aa, Artificial Sequence
  Synthetic peptide
ECRTYRQKGKREEECRRLCEEIRKRENGTVDCQIDGNECEIRACR

SEQ ID NO: 256 - protein, 44 aa, Artificial Sequence
  Synthetic peptide
SCDECYKKMQKTGPPNTEKVKELWKRCQKDESSEYCRRMKKMAK

SEQ ID NO: 257 - protein, 38 aa, Artificial Sequence
  Synthetic peptide
QCYTFRSECTNKEFTVCRPNPEEVEKEARRTKEEECRK

SEQ ID NO: 258 - protein, 45 aa, Artificial Sequence
  Synthetic peptide
QRTRKECDSNNMDECEKRCREEARRKNCRVEIRTRGNKVYCRFEC

SEQ ID NO: 259 - protein, 42 aa, Artificial Sequence
  Synthetic peptide
CEDELRELCKRVGDPKCCEEMKKMLKTGTCDEARKMLEKCLK

SEQ ID NO: 260 - protein, 42 aa, Artificial Sequence
  Synthetic peptide
CCEVTSRSGESRTFCGASRDECEKEAQRCEKEAGVECRWEDK

SEQ ID NO: 261 - protein, 42 aa, Artificial Sequence
  Synthetic peptide
TCHVRCGNITEQTFTTGTCDEMCRKMEEECRKLGGQVDCTSL

SEQ ID NO: 262 - protein, 40 aa, Artificial Sequence
  Synthetic peptide
CKYTFQFCNYDTEQAKEECRKAEEKVKKTHPECEVQCQEC

SEQ ID NO: 263 - protein, 41 aa, Artificial Sequence
  Synthetic peptide
SQETRKKCTEMKKKFKNCEVRCDESNHCVEVRCSDTKYTLC

SEQ ID NO: 264 - protein, 26 aa, Artificial Sequence
  Synthetic peptide
TIKIDCNGEEYKCEDPNRCEEIKRKC

SEQ ID NO: 265 - protein, 36 aa, Artificial Sequence
  Synthetic peptide
PCECDVNGETYTVSSSEECERLCRKLGVTNCRVHCG

SEQ ID NO: 266 - protein, 33 aa, Artificial Sequence
  Synthetic peptide
TCSVTVTGSRSQCEEVQRQLKKKGQPCQVECDN

SEQ ID NO: 267 - protein, 32 aa, Artificial Sequence
  Synthetic peptide
CQTWTFPGCNQTVTECTDEDHKKAREVEKKCG

SEQ ID NO: 268 - protein, 45 aa, Artificial Sequence
  Synthetic peptide
TYCLTVEFTCPRGERYEETFCSDTPEEAKKERKKFETEAEKKCRG

SEQ ID NO: 269 - protein, 30 aa, Artificial Sequence
  Synthetic peptide
CDDVKKEVEEIKKKLTSEDLKKVQEKLDKC

SEQ ID NO: 270 - protein, 37 aa, Artificial Sequence
  Synthetic peptide
CEECKEMARECKEKNQDNCEKTDSQCTYKDNQVKCQS

SEQ ID NO: 271 - protein, 47 aa, Artificial Sequence
  Synthetic peptide
TCEIRVTDTHCKVHCGTQEYKVPPGRTLKVGNCRFTYHDTTCTVECR

SEQ ID NO: 272 - protein, 43 aa, Artificial Sequence
  Synthetic peptide
DCERIRKTVKDLGCSDEMKEKAERCCRGEYNPEECDRELKKCK

SEQ ID NO: 273 - protein, 34 aa, Artificial Sequence
  Synthetic peptide
ADDCKKVQKKVKELNKTNSDDSLKEVKKLQKKCA

SEQ ID NO: 274 - protein, 39 aa, Artificial Sequence
  Synthetic peptide
CVICICGNQEQQTSNTHEKECKEEAEEAERQGCDCKVTT

SEQ ID NO: 275 - protein, 43 aa, Artificial Sequence
  Synthetic peptide
KCEDLRKECRKVGGNPEYEKRIEKMCRDGNDEEAERVARKCKS

SEQ ID NO: 276 - protein, 43 aa, Artificial Sequence
  Synthetic peptide
TCEVRCENGQRIEYPATSDEECERWCRKAKKEFPNYRCTCTHK

SEQ ID NO: 277 - protein, 41 aa, Artificial Sequence
  Synthetic peptide
GCEIRCGNGYTWTVSDNEEKCKRECEKAKKSGCQDVNCTRR

SEQ ID NO: 278 - protein, 38 aa, Artificial Sequence
  Synthetic peptide
CVEKRGSRVHCKAHNKEFQCPPTPDEIERCREECEKRC

SEQ ID NO: 279 - protein, 42 aa, Artificial Sequence
  Synthetic peptide
RCTVELCGRRYECRTDESQLENCAREMQRRVGCPQKPRLECR

SEQ ID NO: 280 - protein, 37 aa, Artificial Sequence
  Synthetic peptide
TCSVTVNTGTPDEDKKECKRVQEEAERKGTQCQCQQE

SEQ ID NO: 281 - protein, 30 aa, Artificial Sequence
  Synthetic peptide
ADDIEKCRKKVEKNSSSQDVQEQLRKCKEA

SEQ ID NO: 282 - protein, 41 aa, Artificial Sequence
  Synthetic peptide
CAQELEDRVRKLEKKLRKKNDDTQVEKLQKKLDELKKRAVC

SEQ ID NO: 283 - protein, 40 aa, Artificial Sequence
  Synthetic peptide
CSYTVRFCYTTEEERKEREERVKKNCKRSGCECRWTNERC

SEQ ID NO: 284 - protein, 37 aa, Artificial Sequence
  Synthetic peptide
CDFNQHGNNMTCNGENDTHCNNDEECKKECEKMKENC

SEQ ID NO: 285 - protein, 38 aa, Artificial Sequence
  Synthetic peptide
TTCVTRRNDDCGQEVTVCSDSEEEARKRAEEILQRRCN

SEQ ID NO: 286 - protein, 36 aa, Artificial Sequence
  Synthetic peptide
CQKDDNGQDCRIDGKHQVECDNDEECCKEIEERACK

SEQ ID NO: 287 - protein, 35 aa, Artificial Sequence
  Synthetic peptide
TCVTVESSCGRRVTVCRPNPEEAEREARKELKKEC

SEQ ID NO: 288 - protein, 46 aa, Artificial Sequence
  Synthetic peptide
PCKEQAKKCYKERPKCNQEELERRVCEAEKRGLDEEEKKKLCNSCD

SEQ ID NO: 289 - protein, 46 aa, Artificial Sequence
  Synthetic peptide
ECERAKEEAKKECSQGSSKEECRERCQEAAKDSDECVEKACQEAAE

SEQ ID NO: 290 - protein, 49 aa, Artificial Sequence
  Synthetic peptide
NCEKLKRKLEKACREGNCDKARKAYEEAQRQNCETDEIRKIYKECEKNC

SEQ ID NO: 291 - protein, 47 aa, Artificial Sequence
  Synthetic peptide
CERCKKKLEECKGSSREDARERCEEAKQESCCSEEERREAEEEKQRA

SEQ ID NO: 292 - protein, 40 aa, Artificial Sequence
  Synthetic peptide
CSTRVTVCNSNDEEAKKIKKRVCEEAKKRGCQCETETCRK

SEQ ID NO: 293 - protein, 43 aa, Artificial Sequence
  Synthetic peptide
EDIQCQSEGYIVVDCGQHQCKFDYDCSDEQQREEAREEAEKCC

SEQ ID NO: 294 - protein, 41 aa, Artificial Sequence
  Synthetic peptide
SEKTRKECEKQREKCGGRPCEYKGPNNCRCEIDGNTYSVDC

SEQ ID NO: 295 - protein, 28 aa, Artificial Sequence
  Synthetic peptide
AEDCERIRKELEKNPNDEIKKKLEKCQA

SEQ ID NO: 296 - protein, 39 aa, Artificial Sequence
  Synthetic peptide
ECVVVCSDGQEQQRQDPCEQVCEEEQRKKGNHDCRCTQT

SEQ ID NO: 297 - protein, 44 aa, Artificial Sequence
  Synthetic peptide
PCDRCARELEEAYPNNPEVNEEARRVKKNCTDEMCKEVKKMKKR

SEQ ID NO: 298 - protein, 41 aa, Artificial Sequence
  Synthetic peptide
DCCVICSGNDQYCAGDNNEEQAEREAKRCEEEGKQYHKYCH

SEQ ID NO: 299 - protein, 43 aa, Artificial Sequence
  Synthetic peptide
SEVRCDGNYCFVIACSGDEQSRDFRCDDEQEKEECKKEAEKEC

SEQ ID NO: 300 - protein, 43 aa, Artificial Sequence
  Synthetic peptide
SDENKKRCETEAKKCKKNGYRVECRNRGTCWEVDCEETTYTIC

SEQ ID NO: 301 - protein, 47 aa, Artificial Sequence
  Synthetic peptide
TCEVRWTNTHCRIKCGTQEYECPPRRRCEIGNFHVDVHDTTCRLHSR

SEQ ID NO: 302 - protein, 35 aa, Artificial Sequence
  Synthetic peptide
CKQRRRYRGSEEECRKYAEELSRRTGCEVEVECET

SEQ ID NO: 303 - protein, 41 aa, Artificial Sequence
  Synthetic peptide
PCCIVYCETQFQHCADTKEKCERQCEEDERQDSQCRSRCTS

SEQ ID NO: 304 - protein, 32 aa, Artificial Sequence
  Synthetic peptide
SCHIDGNQCTYNNTDCNNREECKEYCEKCEKS

SEQ ID NO: 305 - protein, 33 aa, Artificial Sequence
  Synthetic peptide
TCITTTCKGENETKTFCSDDEERIKKESKRCEG

SEQ ID NO: 306 - protein, 39 aa, Artificial Sequence
  Synthetic peptide
TCSETYTFRGNPDECEKRHQELEREAREKGCQFQLECRN

SEQ ID NO: 307 - protein, 32 aa, Artificial Sequence
  Synthetic peptide
ADCDKKLKKVEERSKNGLTEEVQQLRDKVKKC

SEQ ID NO: 308 - protein, 38 aa, Artificial Sequence
  Synthetic peptide
TCKKVTVEGNPDECQEVKKEARKEEEKKGTCVEVECKN

SEQ ID NO: 309 - protein, 36 aa, Artificial Sequence
  Synthetic peptide
ADDCKKLKEKLKKVKKNNGSDEIKKRVEKLRKKCEA

SEQ ID NO: 310 - protein, 42 aa, Artificial Sequence
  Synthetic peptide
RECRINNCREVRFRCPSGQTWTMTVTSCEEAKKMCEKMKKQC

SEQ ID NO: 311 - protein, 43 aa, Artificial Sequence
  Synthetic peptide
CRVECKPGGTCEVHRDSGKREEYTFPTSQDEVCKECKKLQKKC

SEQ ID NO: 312 - protein, 43 aa, Artificial Sequence
  Synthetic peptide
QCERCCEAAKQKNREEAKEACERCQSGDTHEKDAEERCKEAET

SEQ ID NO: 313 - protein, 42 aa, Artificial Sequence
  Synthetic peptide
PCEINSDGCTRQEIPATSPEECKEACERAKKKCTSPVDCQHK

SEQ ID NO: 314 - protein, 43 aa, Artificial Sequence
  Synthetic peptide
PCDEIEKKVRKRGCDPQVEKEVRRVCEEQNDSEQMKQIWKDCS

SEQ ID NO: 315 - protein, 41 aa, Artificial Sequence
  Synthetic peptide
ECTVRCGNQKYRCTTGTCDECAREIEEKCRKLGLEVEIRTL

SEQ ID NO: 316 - protein, 46 aa, Artificial Sequence
  Synthetic peptide
DEAECRIDGNECRLDAKGASDDAREECRELCEEACKKGQKRLQCKR

SEQ ID NO: 317 - protein, 45 aa, Artificial Sequence
  Synthetic peptide
QKETRHCSGQRCEQEARRWCEECKKKGKRVRCRKHGNQVEVQCDK

SEQ ID NO: 318 - protein, 43 aa, Artificial Sequence
  Synthetic peptide
GCEDIDREVEKRGCTEDARRELQKLCKNGQTEDEIRRAADELC

SEQ ID NO: 319 - protein, 47 aa, Artificial Sequence
  Synthetic peptide
QCEVRFTDTHCRVRCGTQEYKLEPGRRVRIGTSEFDVQPTTCTYSHI

SEQ ID NO: 320 - protein, 41 aa, Artificial Sequence
  Synthetic peptide
QCRVICQGHSTTEFSDDSKEECEKECERCEKDGYDSDCHQS

SEQ ID NO: 321 - protein, 48 aa, Artificial Sequence
  Synthetic peptide
ESRCKKSSNTWFCEVGTVQVECPPGRRCTINNQYICEVQGNTCRTENE

SEQ ID NO: 322 - protein, 42 aa, Artificial Sequence
  Synthetic peptide
PCREEAKKRKEEAERKCTTLRVQCPSGCHFEIRCGNQIQEKC

SEQ ID NO: 323 - protein, 44 aa, Artificial Sequence
  Synthetic peptide
NCHEYHGECWYCFVDGDSQFHYHKCDKNAEEAKERKERCERDCS

SEQ ID NO: 324 - protein, 42 aa, Artificial Sequence
  Synthetic peptide
DERDKCAEEIRRECEERGLEVEIRKTDDCVRIRCGTEERTCC

SEQ ID NO: 325 - protein, 43 aa, Artificial Sequence
  Synthetic peptide
EEYRCHGNFVVFYCEQGQEYRCQADCSDEQERERCREEAEKQC

SEQ ID NO: 326 - protein, 39 aa, Artificial Sequence
  Synthetic peptide
ECIICCEGNQCRKFTQEEECKRQAKECEKQGLRYTTIDK

SEQ ID NO: 327 - protein, 44 aa, Artificial Sequence
  Synthetic peptide
SESEKMCRQCEEERKKYPTQETSVRLPKQNCECRVGSTTVDCDC

SEQ ID NO: 328 - protein, 41 aa, Artificial Sequence
  Synthetic peptide
CRYEKETRGDDEQCRKEKEKLCEEAKKEEPRCQCHFRCQKG

SEQ ID NO: 329 - protein, 48 aa, Artificial Sequence
  Synthetic peptide
QCEEYARELREEAERQNCEEAREKAEECEEKNDCECAKEAEEKLRECS

SEQ ID NO: 330 - protein, 40 aa, Artificial Sequence
  Synthetic peptide
REEEVKKCCKEWHRRMKPDTFQVRTREGKCTVSRGRTYQC

SEQ ID NO: 331 - protein, 42 aa, Artificial Sequence
  Synthetic peptide
EEERRCAEECCQQFSQKEECCERCEECANQQERAEKAKKDAC

SEQ ID NO: 332 - protein, 46 aa, Artificial Sequence
  Synthetic peptide
ECYKEYCQEIKECQSTSEEEAEERAREACNTSCEEARKKAEEACQS

SEQ ID NO: 333 - protein, 44 aa, Artificial Sequence
  Synthetic peptide
QCFEVEVNCPDKNQSFRYRFCSSNPEEAERRAREAEKRARENCK

SEQ ID NO: 334 - protein, 49 aa, Artificial Sequence
  Synthetic peptide
GSTCEIRVTDTHCKVHCGTQEYKVPPGRTLKVGNCRFTYHDTTCTVECR

SEQ ID NO: 335 - protein, 49 aa, Artificial Sequence
  Synthetic peptide
GSGCEIRVTSQYCEVRCGTQKYKVPPGRTLKVGNCRFTYHDTTCTVECR

SEQ ID NO: 336 - protein, 49 aa, Artificial Sequence
  Synthetic peptide
GSGCEIYVHSQYCRVRCGTQEYKVPPGRTLKVGNCRFTYHDTTCTVECR

SEQ ID NO: 337 - protein, 45 aa, Artificial Sequence
  Synthetic peptide
GSTCEVRCENGQRIEYPATSDEECERWCRKAKKEFPNYRCTCTHK

SEQ ID NO: 338 - protein, 46 aa, Artificial Sequence
  Synthetic peptide
GSAPCKVYCENGQEIYYPATSDEECERWCREAKKRFPNYDCQCTRA

SEQ ID NO: 339 - protein, 46 aa, Artificial Sequence
  Synthetic peptide
GSAPCEVYCEDGQTIRYPATSDEECERWCREAKKRFPNYDCTCTRA

SEQ ID NO: 340 - protein, 51 aa, Artificial Sequence
  Synthetic peptide
GSNCEKLKRKLEKACREGNCDKARKAYEEAQRCNCETDEIRKIYKECEKNC

SEQ ID NO: 341 - protein, 51 aa, Artificial Sequence
  Synthetic peptide
GSNCDKLRDKLEKACREGYCDKARKAYKEAQDCNCHTDEIEKIYRECEKNC

SEQ ID NO: 342 - protein, 51 aa, Artificial Sequence
  Synthetic peptide
GSNCDELREKLRKACEEGYCDKARKAYEEAQRCNCHTDEIEKIYRECEKNC

(19) United States
(12) Patent Application Publication
(10) Pub. No.: US 2018/0068054 A1
(43) Pub. Date: Mar. 8, 2018

(54) Hyperstable Constrained Peptides and Their Design

(71) Applicants: University of Washington, Seattle, WA (US); University of Queensland, St. Lucia (AU)

(72) Inventors: David BAKER, Seattle, WA (US); Christopher BAHL, Seattle, WA (US); Jason GILMORE, Seattle, WA (US); Gaurav BHARDWAJ, Seattle, WA (US); Vikram K. MULLIGAN, Seattle, WA (US); Peta HARVEY, St. Lucia (AU); Olivier CHENEVAL, St. Lucia (AU); David CRAIK, St. Lucia (AU)

(21) Appl. No.: 15/696,889

(22) Filed: Sep. 6, 2017

Related U.S. Application Data
(60) Provisional application No. 62/383,721, filed on Sep. 6, 2016.
(60) Provisional application No. 62/383,733, filed on Sep. 6, 2016.

(51) Int. Cl. G06F 19/16; C07K 14/00; C07K 7/08; G01N 33/68

(52) U.S. Cl. CPC G06F 19/16; G01N 33/6818; C07K 7/08; C07K 14/00

(57) ABSTRACT

Hyperstable constrained peptides and methods and apparatus for designing such peptides are provided. A computing device can determine a peptide backbone using a computing device. The computing device can place zero or more disulfide bonds in the peptide backbone. The computing device can design one or more peptide sequences based on the peptide backbone. The computing device can validate at least one validated peptide sequence of the one or more peptide sequences. An output can be generated based on the at least one validated peptide sequence.

### CROSS-REFERENCE TO RELATED-APPLICATIONS

The present application claims priority to U.S. Provisional Patent Application No. 62/383,721 entitled “Accurate de novo design of Hyperstable Constrained Peptides”, filed Sep. 6, 2016 and to 62/383,733 entitled “De novo Design of Heterochiral Constrained Peptides with Non-canonical Backbones and Sequences”, filed Sep. 6, 2016, all of which are entirely incorporated by reference herein for all purposes.

### BACKGROUND

The vast majority of drugs currently approved for use in humans are either proteins or small molecules. Lying between the two in size, and integrating the advantages of both constrained peptides are an underexplored frontier for drug discovery. Naturally-occurring constrained peptides, such as conotoxins, chlorotoxin, knottins, and cyclotides, play critical roles in signaling, virulence and immunity, and are among the most potent pharmacologically active compounds known. These peptides are constrained by disulfide bonds or backbone cyclization to favor binding-competent conformations that precisely complement their targets. Inspired by the potency of these compounds, there have been considerable efforts to generate new bioactive molecules by re-engineering existing constrained peptides using loop grafting, sequence randomization, and selection. These approaches are hindered by the limited variety of naturally-occurring constrained peptide structures and the inability to achieve global shape complementarity with targets.

### SUMMARY

Naturally occurring, pharmacologically active peptides constrained with covalent crosslinks generally have shapes evolved to fit precisely into binding pockets on their targets. Such peptides can have excellent pharmaceutical properties, combining the stability and tissue penetration of small molecule drugs with the specificity of much larger protein therapeutics. The ability to design constrained peptides with precisely specified tertiary structures would enable the design of shape-complementary inhibitors of arbitrary targets. Computational methods for de novo design of conformationally-restricted peptides are described herein, and the use of these methods to design 15-50 residue disulfide-crosslinked and heterochiral N—C backbone-cyclized peptides. These peptides are exceptionally stable to thermal and chemical denaturation, and twelve experimentally-determined X-ray and NMR structures are nearly identical to the computational models. The computational design methods and stable scaffolds presented here provide the basis for development of a new generation of peptide-based drugs.

In one aspect, a method is provided. A computing device determines a peptide backbone. The computing device places one or more disulfide bonds in the peptide backbone. The computing device designs one or more peptide sequences based on the peptide backbone. The computing device validates at least one validated peptide sequence of the one or more peptide sequence. An output is generated that is based on the at least one validated peptide sequence.

In another aspect, a computing device is provided. The computing device includes one or more processors; and a non-transitory computer-readable medium that is configured to store at least computer-readable instructions that, when executed by the one or more processors, cause the computing device to perform functions. The functions include: determining a peptide backbone; placing one or more disulfide bonds in the peptide backbone; designing one or more peptide sequences based on the peptide backbone; validating at least one validated peptide sequence of the one or more peptide sequences; and generating an output based on the at least one validated peptide sequence.

In another aspect, a non-transitory computer-readable medium is provided. The non-transitory computer-readable medium is configured to store at least computer-readable instructions that, when executed by one or more processors of a computing device, cause the computing device to perform functions. The functions include: determining a peptide backbone; placing one or more disulfide bonds in the peptide backbone; designing one or more peptide sequences based on the peptide backbone; validating at least one validated peptide sequence of the one or more peptide sequences; and generating an output based on the at least one validated peptide sequence.

In another aspect, a device is provided. The device includes means for determining a peptide backbone; means for placing one or more disulfide bonds in the peptide backbone; means for designing one or more peptide sequences based on the peptide backbone; means for validating at least one validated peptide sequence of the one or more peptide sequences; and means for generating an output based on the at least one validated peptide sequence.

In a further aspect, the invention provides non-naturally occurring polypeptides comprising

(a) 2-6 secondary structure domains, wherein each secondary structure domain is either a β-sheet (E domain) of between 4-9 amino acid residues in length, or an α-helix (H domain) of between 4-15 amino acid residues in length;

(b) a loop of 2-5 amino acid residues in length connecting adjacent secondary structure domains;

wherein the polypeptide is between 15-50 amino acid residues in length.

In one embodiment, the polypeptide includes at least two cysteine residues capable of forming a disulfide bond. In another embodiment, the at least two cysteine residues capable of forming on a disulfide bond are present on separate secondary structure domains. In a further embodiment, the polypeptide comprises a secondary structure domain arrangement selected from the group consisting of HH, EE, HHH, EHE, EEH, HEE, HEEE, EEHE, EHEE, EEEH, and EEEEEE.

In one embodiment, the polypeptide is non-cyclic. In another embodiment, the polypeptide does not include any D-amino acid residues. In a further embodiment, each E domain is between 4-9 amino acid residues in length, each H domain is between 9-15 amino acid residues in length, and each loop is between 2-5 amino acid residues in length. In another embodiment, each E domain and each H domain includes at least one non-polar amino acid other than alanine. In another embodiment, proline residues are not present within the interior of any secondary structure domain. In a further embodiment, the polypeptide includes 2-8 cysteine residues capable of forming disulfide bonds. In another embodiment, the polypeptide includes 1-4 disulfide bonds, wherein the disulfide bonds bind cysteine pairs that are separated by at least 5 amino acids in the primary amino acid sequence of the polypeptide. In one embodiment, each disulfide bond binds a first cysteine residue present in a first secondary structure domain to a second cysteine residue present in a second secondary structure domain.

In another embodiment, the polypeptide includes 1 or more D-amino acid residues. In one embodiment, each E domain is between 4-6 amino acid residues in length, each H domain is between 4-14 amino acid residues in length, and each loop is between 2-4 amino acid residues in length. In another embodiment, the polypeptide is 18-32 amino acids in length. In a further embodiment, the polypeptide comprises a secondary structure domain arrangement selected from the group consisting of EHE, EEH, and HEE. In one embodiment, the polypeptide includes at least 4 cysteine residues capable of forming disulfide bonds. In another embodiment, the polypeptide includes at least two disulfide bonds. In one embodiment, each disulfide bond binds a first cysteine residue present in a first secondary structure domain to a second cysteine residue present in a second secondary structure domain.

In another embodiment, the polypeptide comprises a peptide bond linking the terminal amino acid residues. In one embodiment, each E domain is between 4-6 amino acid residues in length, each H domain is between 4-14 amino acid residues in length, and each loop is between 2-4 amino acid residues in length. In another embodiment, the polypeptide is 18-32 amino acids in length. In a further embodiment, the polypeptide includes 1 or more D-amino acid residues. In another embodiment, the polypeptide comprises a secondary structure domain arrangement selected from the group consisting of H_{R}H_{R}, H_{L}H_{R}, EE, and HHH, wherein H_{R} is a right handed α-helix, and H_{L} is a left-handed α-helix. In one embodiment, the polypeptide includes at least 2 cysteine residues capable of forming disulfide bonds. In another embodiment, the polypeptide includes at least one disulfide bond. In a further embodiment, each disulfide bond binds a first cysteine residue present in a first secondary structure domain to a second cysteine residue present in a second secondary structure domain.

In one embodiment, the polypeptide is at least 30% identical along its entire length to the amino acid sequence of any one of SEQ ID NOS: 1-333.

In another aspect, the invention provides an isolated nucleic acid encoding the polypeptide of any embodiment or combination of embodiments of the invention. In another embodiment, the invention provides a recombinant expression vector comprising the isolated nucleic acid of any embodiment or combination of embodiments of the invention operatively linked to a promoter. In a further embodiment, the invention provides a recombinant host cell comprising the recombinant expression vector of any embodiment of the invention.

### BRIEF DESCRIPTION OF THE DRAWINGS

The following figures are in accordance with example embodiments.

FIG. 1: Designed peptide topologies. The designed secondary structure architectures for each of the three classes of constrained peptides (genetically-encodable disulfide-rich, heterochiral disulfide-crosslinked, and cyclic) span most of the topologies that can be formed with four or fewer secondary structure elements. Arrows: β-strands, orange cylinders: right-handed α-helices, green cylinder: left-handed α-helix; red: loop segments containing D-amino acid residues.

FIG. 2: Computational design and biophysical characterization of genetically-encodable disulfide-rich peptides. Genetically-encodable peptides are given the prefix “g” and a number to differentiate designs that share a common topology. (column a) Cartoon renderings of each design are shown with rainbow coloring from the N-terminus (blue) to the C-terminus (red), and disulfide bonds are shown as sticks. (column b) The energy landscape of each designed sequence was assessed by Rosetta™ structure prediction calculations starting from an extended chain (blue dots) or from the design model (orange dots); lower energy structures were sometimes sampled in the former because disulfide constraints were only present in the latter. (column c) CD spectra at 20° C. (blue line), after heating to 95° C. (red line), and upon cooling back to 20° C. (green line). Spectra collected with 2.5 mM TCEP are shown in purple. (column d) CD steady-state wavelength spectra as a function of GdnHCl concentration.

FIG. 3: X-ray crystal structures and NMR solution structures of designed peptides are very close to design models. Structures for gEHE_06, gEEH_04, gEEHE_02, and gHHH_06 were determined by NMR spectroscopy, and the structure of gEHEE_06 was determined by X-ray crystallography. (column a) C_{α} traces of NMR ensembles, or superimposed members of the asymmetric unit, (grey) are aligned against the design model (rainbow). Disulfide bonds are shown with sidechain atoms rendered as sticks with sulfur atoms colored yellow. (column b) A cartoon representation of the lowest energy conformer of each NMR ensemble or crystallographic asymmetric unit (grey) is shown aligned to the design model (rainbow). Sidechain atoms of hydrophobic core residues are rendered as sticks.

FIG. 4: Design and characterization of heterochiral disulfide-constrained peptides The prefix “NC” denotes non-canonical sequence or backbone architecture, and a numerical suffix differentiates designs sharing a common topology. (Column a) Cartoon representations of design models with the N-terminus in blue and C-terminus in red. (Column b) Folding energy landscapes from Rosetta™ ab initio structure prediction calculations. Blue dots indicate lowest-energy structures identified in independent Monte Carlo trajectories. Orange dots are from trajectories starting with the design model. (r.e.u: Rosetta™ Energy Units, RMSD: root mean square deviation from the designed topology). (Column c) Five representative trajectories from a total of 50 independent molecular dynamics simulations starting from the design model with different initial velocities. (Column d) NMR-determined structure ensembles. Cartoon representations colored and oriented as in column a. (Column e) Superposition of the designed structure (blue) with the lowest-energy NMR structure (green). (Column f) CD wavelength spectra between 195 nm and 260 nm recorded at 25° C. (black), 55° C. (blue), 95° C. (red), and after cooling back to 25° C. (green). (Column g) CD spectra recorded at 0 M (black), 2 M (blue), 4 M (green), or 6 M GdnHCl (red), or with 2.5 mM TCEP/0 M GdnHCl (purple). Data are truncated in the far-UV region for spectra acquired in the presence of high GdnHCl concentrations (due to GdnHCl absorbance).

FIG. 5: Design and characterization of N—C backbone cyclic peptides Columns are as indicated for FIG. 4. A lowercase “c” in the peptide name indicates N—C cyclic backbone.

FIG. 6: Design and characterization of a peptide with non-canonical secondary and tertiary structure. a) NC_H_{L}H_{R}_{_}D1 design (cyan: L-amino acids, orange: D-amino acids) b) Folding energy landscape generated using a new structure prediction algorithm compatible with non-canonical secondary structures. c) Five representative molecular dynamics trajectories (from a total of 50) starting from the design model with different initial velocities. d) NMR-determined structure ensembles, colored and oriented as in first panel. e) Superposition of designed structure (blue) with lowest-energy NMR structure (green). f) CD spectra between 195 nm and 260 nm recorded at 25° C. (black), 55° C. (blue), 95° C. (red), and after cooling back to 25° C. (green). The CD spectrum of NC_H_{L}H_{R}_{_}D1 exhibits very weak signals because the L- and D-helical signals largely cancel. g) Secondary ¹H_{α} chemical shifts (ppm) show no change from 25° C. (black) to 75° C. (red) (SEQ ID NO:09).

FIG. 7 Disulfide bonds are well defined by X-ray crystallography. An Fₒ−F_{c} omit-map is shown contoured at 4σ for design gEHEE_06. Disulfide sulfur atoms were removed, and the omit-map was calculated following real-space refinement.

FIG. 8: Sidechain placement in non-canonical peptide designs chosen for experimental characterization. Designs are shown as cartoon and stick representations (top row in each box) and as van der Waals spheres showing sidechain packing (bottom row in each box). L-amino acid residues are shown in cyan, and D-amino acid residues are colored orange. Sidechains of D- or L-variants of alanine, phenylalanine, isoleucine, leucine, valine, tryptophan, and tyrosine are colored grey to aid visualization of hydrophobic packing interactions.

FIG. 9: Molecular dynamics screening of designed peptides. Fifty independent molecular dynamics (MD) simulations in explicit solvent conditions, all starting from the designed peptide, were used for discriminating good, kinetically-stable (e.g. ERE_D1) designs from non-optimal designs of the same topology (e.g. ERE_X18 and ERE_X11). a) Five representative trajectories from MD simulation runs. Designs that showed good convergence, and smaller fluctuations were selected for further experimental characterization. b) RMSD distribution from all 50 trajectories. Only the last one-third of the trajectory was used for this analysis. Designs with narrower distributions were picked for further testing. c) Concatenated trajectory of all 50 independent runs shows lower fluctuations for the more optimal designs.

FIG. 10: Structural characterization of NC_EEH_D1. NMR structure of NC_EEH_D1 does not match the designed topology. a) Rosetta™-designed model for NC_EEH_D1. b) Ensemble of conformers representing the NMR solution structure. c) Superposition of the designed model (blue) with a representative NMR conformer (green).

FIG. 11: Structural mapping of sequence-aligned region between NC_EHE_D1 and 2MA5. Design NC_EHE_D1 and PDB entry 2MA5 show weak but significant (e-value: 2×10⁻⁴) sequence alignment, which is highlighted in purple. The aligned region folds into very different structures in the different contexts of peptide and protein.

FIG. 12: Mutational tolerance of selected genetically-encodable designs. RP-HPLC traces for the parental designs are shown next to the redesigned variants where applicable. Proteins run under oxidized conditions are shown in black while proteins run following reduction with 10 mM DTT are shown in red. Insets within each panel are shown only to highlight the SDS-PAGE mobility of each purified protein under oxidizing (left band) and reducing conditions (right band). Sequence alignments are shown with the mutated positions highlight in red, along with theoretical isoelectric points as calculated by ProtParam (Sequences from the sequence alignments are: EEE_EEE_1.1_02 is SEQ ID NO:334; EE_EEE_1.1_02_0002 is SEQ ID NO:335; EE_EEE_1.1_02_0003 is SEQ ID NO:336; EEHE_2.1_02 is SEQ ID NO:337; EEHE_2.1_02_0005 is SEQ ID NO:338; EEHE_2.1_02_0008 is SEQ ID NO:339; HHH_3.0_06 is SEQ ID NO:340; HHH_3.0_06_0005 is SEQ ID NO:341; HHH_3.0_06_0008 is SEQ ID NO:342).

FIG. 13: Mutational tolerance of selected NC designs. α-b) Mutational tolerance of D-proline, L-proline loop of design NC_cEE_D1 (green in panel a), assessed by secondary ¹H_{α} chemical shift for the design sequence (black bars in panel b) (SEQ ID NO:05) and the p18d loop mutation (red bars). Eliminating this key proline residue does not result in loss of β-strand signal. c-d) Mutational tolerance of loop region of design NC_HEE_D1 (green in panel c), as assessed by CD spectroscopy for the design sequence (left plot, panel d) and for the D19T, p20q, P21D triple mutant (right plot, panel d). Both proline residues may be mutated without loss of secondary structure or major change in the thermal stability. e-g) computationally predicted mutational tolerance of design NC_H_{L}H_{R}_{_}D1, across the entire sequence. Each position was successively mutated in silico to D- or L-alanine, arginine, aspartate, phenylalanine, or valine (preserving the position's chirality), and full folding simulations were carried out with the Rosetta™ simple_cycpep_predict application. Folding funnel quality was evaluated using the Pₙₑₐᵣ metric. e) Representative plots of energy vs. RMSD from the design structure, plotted for the design sequence (top), for the non-disruptive R14F mutation (middle), and for the e18v mutation (bottom). Results from generalized kinematic loop closure (GenKIC)-based structure prediction runs are shown in blue, and relaxation runs, in orange. Note that the bottom case shows many sampled states far from the design state with energy equal to or less than the design state energy. f) Mutational tolerance by position (vertical axis) and mutation (horizontal axis). Blue rectangles represent well-tolerated mutations, and red to black rectangles represent disruptive mutations, based on Pₙₑₐᵣ evaluation of the folding funnel. Black borders indicate the design sequence. g) Mutational tolerance mapped onto the NC_H_{L}H_{R}_{_}D1 structure, with colors as in the previous panel. Most positions tolerate mutation well, with only the disulfide bridge (C8-c21) and the salt bridges formed by e18 being highly sensitive. The hydrogen bond networks formed by residues Q5, e24, and s25 show some moderate sensitivity to mutation, as do residues E3 and e16.

FIG. 14: The ¹H-¹⁵N HSQC spectrum for gEHE_06 (˜1 mM) collected at a proton resonance frequency of 500 MHz, 20° C., in 50 mM sodium chloride, 25 mM sodium acetate, pH 4.8. The wide chemical shift dispersion of the amide resonances in the nitrogen and proton dimension is characteristic of a structured protein.

FIG. 15: The ¹H-¹⁵N HSQC spectrum for gEEHE_02 (˜0.5 mM) collected at a proton resonance frequency of 500 MHz, 20° C. in 50 mM sodium chloride, 25 mM sodium acetate, pH 4.8. The wide chemical shift dispersion of the amide resonances in the nitrogen and proton dimension is characteristic of a structured protein.

FIG. 16: The ¹H-¹⁵N HSQC spectrum for gHHH_06 (˜1 mM) collected at a proton resonance frequency of 750 MHz, 20° C., 50 mM sodium phosphate, pH 6.0, 4 μM 4,4-dimethyl-4-silapentane-1-sulfonic acid salt, 0.02% sodium azide with the backbone amide resonances labeled. The side chain Asn, Gln, and Gln resonances are labeled with an asterisk.

FIG. 17: The ¹H-¹⁵N HSQC spectrum for gEEH_04 (1 mM) collected at a proton resonance frequency of 750 MHz, 20° C., 50 mM sodium phosphate, pH 6.0, 4 μM 4,4-dimethyl-4-silapentane-1-sulfonic acid, 0.02% sodium azide with the backbone amide resonances labeled. The side chain Asn, Gln, and Gln resonances are labeled with an asterisk.

FIG. 18: NMR spectroscopy analysis of designed non-canonical peptides. a) Proton NMR spectra for each of the seven designed topologies recorded at a ¹H resonance frequency of 600 MHz, 25° C. Spectra are well-dispersed and sharp, consistent with folded proteins. b) Secondary ¹H_{α} chemical shifts (in ppm) for each of the seven designed topologies.

FIG. 19: Secondary ¹H_{α} chemical shifts at a range of temperatures for peptide NC_cH_{L}H_{R}_{_}D1 (SEQ ID NO:09). NMR spectra were collected at 25° C. (black bars), 55° C. (blue bars), 75° C. (red bars), and again after cooling to 25° C. (green bars). Secondary chemical shifts are largely unchanged during heating, showing clear alpha-helical signatures for residues 2-11 (the designed α_{R}-helix) and residues 16-25 (the designed α_{L}-helix), indicating no significant loss of secondary structure resulting from heating. Secondary chemical shifts are identical to the original values after cooling, indicating that the peptide is also not aggregation-prone or otherwise prone to irreversible conformation changes on heating. Overall, these results indicate considerable thermostability.

FIG. 20: Flowchart of a method for designing non-canonical cyclic peptides. The flowchart illustrates a combined fragment assembly-based design pipeline and a fragment-free GenKIC-based design pipeline. Final computational validation was carried out using MD simulations and fragment-based Rosetta™ ab initio structure prediction. For peptides containing isolated D-amino acids, these residues were mutated to glycine for Rosetta™ ab initio structure prediction. The GenKIC-based design pipeline permits design of non-canonical topologies like the mixed αLαR topology, which occurs in no known natural protein.

FIG. 21: Flowchart of a method for a generalized kinematic closure technique. GenKIC permits the sampling of closed conformations of arbitrary chains of atoms. These chains can pass through canonical or non-canonical backbone or sidechain linkages. Bond length, bond angle, and torsional degrees of freedom in the chain can be fixed, perturbed from a starting value by small amounts, set to user-defined values, or sampled randomly, as the user sees fit. The algorithm then solves for six torsion angles adjacent to three user-defined pivot atoms in order to enforce closure of the loop. The many solutions from the closure are then filtered internally, and each can be subjected to arbitrary user-defined Rosetta™ protocols and filtration in order to further prune the solution list. A single solution is selected from those passing filters by user-defined selection criteria. This flowchart shows the steps in a single invocation of the algorithm; for sampling, a user may specify that the algorithm be applied any number of times.

FIGS. 22A and 22B: Flowchart of a method for structure prediction using generalized kinematic closure. GenKIC allows sampling of closed conformations of arbitrary chains of atoms, passing through canonical or non-canonical backbone or sidechain linkages. Bond length, bond angle, and torsional degrees of freedom in the chain can be fixed, perturbed from a starting value by small amounts, set to user-defined values, or sampled randomly. The algorithm then solves for six torsion angles adjacent to three user-defined pivot atoms in order to enforce closure of the loop. The many solutions from the closure are then filtered internally, and each can be subjected to arbitrary user-defined Rosetta™ protocols and filtration in order to prune the solution list further. A single solution is selected from those passing filters by a user-defined selection criterion. This flowchart shows the steps in a single invocation of the algorithm; for sampling, a user may specify that the algorithm be applied any number of times. User inputs are shown in blue, steps carried out by the GenKIC algorithm itself are in green, steps carried out by Rosetta™ code external to the GenKIC algorithm are shown in yellow, and outputs are shown in salmon.

FIG. 22C: Images related to the method for structure prediction using generalized kinematic closure of FIGS. 22A and 22B. b) The initial, random peptide conformation with bad terminal peptide bond geometry. c) Ensemble of closed conformations found for a single closure attempt. In this example, residue 7 (cyan) is the fixed anchor residue. Certain regions of the peptide have been set to left- or right-handed helical conformations prior to solving closure equations. d) A single closed solution with relative cysteine sidechain orientations that pass the initial, low-stringency filter for disulfide (fa_dslj) conformational energy. e) The resulting structure, following sidechain repacking, energy-minimization, and cyclic de-permutation.

FIG. 23: A block diagram of an example computing network.

FIG. 24A: A block diagram of an example computing device.

FIG. 24B: A block diagram of an example network of computing devices arranged as a cloud-based server system.

FIG. 25: A flowchart of a method.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In one aspect, the invention provides non-naturally occurring polypeptides comprising or consisting of:

(a) 2-6 secondary structure domains, wherein each secondary structure domain is either a β-sheet (E domain) of between 4-9 amino acid residues in length, or an α-helix (H domain) of between 4-15 amino acid residues in length;

(b) a loop of 2-5 amino acid residues in length connecting adjacent secondary structure domains;

wherein the polypeptide is between 15-50 amino acid residues in length.

As demonstrated in the examples, the inventors have developed computational methods for de novo design of conformationally-restricted peptides, and the use of these methods to design a large number of exemplary 15-50 residue constrained peptides. These peptides are exceptionally stable to thermal and chemical denaturation, and experimentally-determined X-ray and NMR structures are nearly identical to the computational models. The hyperstable polypeptides disclosed herein provide robust starting scaffolds for generating peptides that bind targets of interest using computational interface design or experimental selection methods. Solvent-exposed hydrophobic residues can be introduced without impairing folding or solubility, suggesting high mutational tolerance. Hence it should be possible to reengineer the peptide surfaces, incorporating target-binding residues to construct binders, agonists, or inhibitors.

As used herein, a β-sheet secondary structure domain comprises β strands connected laterally by backbone hydrogen bonds, as is understood by those of skill in the art. As used herein, an α-helix secondary structure domain is a right-handed or left-handed (when D amino acids are involved) helix in which backbone amine groups donate a hydrogen bond to backbone carbonyl groups of amino acids 3-4 residues before it along the primary amino acid sequence of the polypeptide, as is understood by those of skill in the art.

In various embodiments, the polypeptide comprises or consists of 2-6, 2-5, 2-4, 2-3, 3-6, 3-5, 3-4, 4-6, 4-5, 5-6, 2, 3, 4, 5, or 6 secondary structure domains. In various non-limiting embodiments, the secondary structure arrangement of the polypeptide may be selected from the group consisting of HH, EE, HHH, EHE, EEH, HEE, HEEE, EEHE, EHEE, EEEH, and EEEEEE, wherein H is a helix and E is a beta strand.

In various embodiments, each E domain is independently between 4-9, 4-8, 4-7, 4-6, 4-5, 5-9, 5-8, 5-7, 5-6, 6-9, 6-8, 6-7, 7-9, 7-8, 8-9, 4, 5, 6, 7, 8, or 9 amino acid residues in length. In one embodiment, each E domain in the polypeptide is the same length; in another embodiment, not all E domains in the polypeptide are the same length. In other embodiments, each H domain is independently between 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, 14-15, 4, 5, 6, 7, 8, 9, 10, 11, 12, 13, 14, or 15 amino acid residues in length. In one embodiment, each H domain in the polypeptide is the same length; in another embodiment, not all H domains in the polypeptide are the same length. In further embodiments, each loop is independently 2-5, 2-4, 2-3, 3-5, 3-4, 4-5, 2, 3, 4, or 5 amino acids in length. In one embodiment, each loop in the polypeptide is the same length; in another embodiment, not all loops in the polypeptide are the same length.

As used throughout the present application, the term “polypeptide” is used in its broadest sense to refer to a sequence of subunit amino acids. The polypeptides of the invention may comprise glycine, L-amino acids, D-amino acids (which are resistant to L-amino acid-specific proteases in vivo), or a combination of glycine and D- and L-amino acids. As disclosed herein, L-amino acids and glycine are shown in upper case letters, and D-amino acids are shown in lower case letters.

In another embodiment, the polypeptide includes at least two cysteine residues capable of forming a disulfide bond. In this embodiment, a disulfide bond can form between a pair of cysteine residues; the polypeptide may have multiple pairs of cysteine residues capable for forming disulfide bonds. In various embodiments, the polypeptide may have 1, 2, 3, 4, 5, or more pair of cysteine residues capable of forming 1, 2, 3, 4, or 5 disulfide bonds. In one embodiment, each member of a given pair of cysteine residues capable of forming a disulfide bond is present on separate secondary structure domains. In other embodiments, each member of a given pair of cysteine residues capable of forming a disulfide bond is present on the same secondary structure domain.

In a further embodiment, the polypeptide is non-cyclic. In one embodiment, the non-cyclic polypeptide does not include any D-amino acid residues (i.e.: it contains L-amino acid residues and may contain glycine residues). In a further embodiment of non-cyclic polypeptides of the invention, each E domain is between 4-9 amino acid residues in length, each H domain is between 9-15 amino acid residues in length, and each loop is between 2-5 amino acid residues in length. Variations on these embodiments of the length of the secondary structure domains and loops are provided above. In another embodiment, each E domain and each H domain includes at least one (i.e.: 1, 2, 3, or more) non-polar amino acid other than alanine (i.e.: Val (V), Leu (L), Ile (I), Pro (P), Phe (F), Trp (W), or Met (M)) to direct folding to the polypeptide core. In a further embodiment, proline residues are not present within the interior of any secondary structure domain; in this embodiment proline residues may only be present in the loop(s) or in the secondary structure domains as the first or last residue in an E or H domain. In a further embodiment, the polypeptide includes 2-8 cysteine residues capable of forming disulfide bonds; in this embodiment, the polypeptide may further include 1-4 disulfide bonds. In a further embodiment, the disulfide bonds bind cysteine pairs that are separated by at least 5 amino acids in the primary amino acid sequence of the non-cyclic polypeptide. In still further embodiment, each disulfide bond binds a first cysteine residue present in a first secondary structure domain to a second cysteine residue present in a second secondary structure domain. In various further embodiments, the polypeptide is 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acid residues in length.

In another embodiment, the polypeptide includes 1 or more (i.e.: 1, 2, 3, 4, 5, 6, 7, 8, or more) D-amino acid residues. In one embodiment, each E domain is between 4-6 amino acid residues in length, each H domain is between 4-14 amino acid residues in length, and each loop is between 2-4 amino acid residues in length. In another embodiment, each E domain may independently include 1-6, 2-6, 3-6, 4-6, 5-6, 1-5, 2-5, 3-5, 4-5, 1-4, 2-4, 3-4, 1-3, 2-3, 1-2, 1, 2, 3, 4, 5, or 6 D-amino acids. In a further embodiment, each H domain may independently include 1-14, 1-13, 1-12, 1-11, 1-10, 1-9, 1-8, 1-7, 1-6, 1-5, 1-4, 1-3, 1-2, 2-14, 2-13, 2-12, 2-11, 2-10, 2-9, 2-8, 2-7, 2-6, 2-5, 2-4, 2-3, 3-14, 3-13, 3-12, 3-11, 3-10, 3-9, 3-8, 3-7, 3-6, 3-5, 3-4, 4-14, 4-13, 4-12, 4-11, 4-10, 4-9, 4-8, 4-7, 4-6, 4-5, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, 13-14, 4, 5, 6, 7, 8, 9, 10, 11, 12, 13, or 14 D amino acid residues. In another embodiment, each loop may independently include 1-4, 1-3, 1-2, 2-4, 2-3, 3-4, 1, 2, 3, or 4 D amino acids. In a further embodiment, the polypeptide is 18-32 amino acids in length; in various further embodiments, the polypeptide is 18-30, 18-28, 18-25, 18-22, 18-20, 20-32, 20-30, 20-28, 20-25, 20-22, 22-32, 22-30, 22-25, 25-32, 25-30, 25-28, 28-32, 28-30, or 30-32 amino acids in length. In another embodiment, the polypeptide comprises a secondary structure domain arrangement selected from the group consisting of EHE, EEH, and HEE. In a further embodiment, the polypeptide includes at least 4 cysteine residues capable of forming disulfide bonds. In another embodiment, the polypeptide includes at least two disulfide bonds; in one such embodiment, each disulfide bond may bind a first cysteine residue present in a first secondary structure domain to a second cysteine residue present in a second secondary structure domain.

In another embodiment, the polypeptide comprises a peptide bond linking the terminal amino acid residues (i.e.: the polypeptide is cyclic). In one such embodiment, each E domain is between 4-6 amino acid residues in length, each H domain is between 4-14 amino acid residues in length, and each loop is between 2-4 amino acid residues in length. Variations on these embodiments of the length of the secondary structure domains and loops are provided above. In a further embodiment, the polypeptide is 18-32 amino acids in length; in various further embodiments, the polypeptide is 18-30, 18-28, 18-25, 18-22, 18-20, 20-32, 20-30, 20-28, 20-25, 20-22, 22-32, 22-30, 22-25, 25-32, 25-30, 25-28, 28-32, 28-30, or 30-32 amino acids in length. In another embodiment, the polypeptide includes 1 or more D-amino acid residues.. In another embodiment, each E domain may independently include 1-6, 2-6, 3-6, 4-6, 5-6, 1-5, 2-5, 3-5, 4-5, 1-4, 2-4, 3-4, 1-3, 2-3, 1-2, 1, 2, 3, 4, 5, or 6 D-amino acids. In a further embodiment, each H domain may independently include 1-14, 1-13, 1-12, 1-11, 1-10, 1-9, 1-8, 1-7, 1-6, 1-5, 1-4, 1-3, 1-2, 2-14, 2-13, 2-12, 2-11, 2-10, 2-9, 2-8, 2-7, 2-6, 2-5, 2-4, 2-3, 3-14, 3-13, 3-12, 3-11, 3-10, 3-9, 3-8, 3-7, 3-6, 3-5, 3-4, 4-14, 4-13, 4-12, 4-11, 4-10, 4-9, 4-8, 4-7, 4-6, 4-5, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, 13-14, 4, 5, 6, 7, 8, 9, 10, 11, 12, 13, or 14 D amino acid residues. In another embodiment, each loop may independently include 1-4, 1-3, 1-2, 2-4, 2-3, 3-4, 1, 2, 3, or 4 D amino acids. In another embodiment, the polypeptide comprises a secondary structure domain arrangement selected from the group consisting of H_{R}H_{R}, H_{L}H_{R}, EE, and HHH, wherein H_{R} is a right handed α-helix, and H_{L} is a left-handed α-helix. In a further embodiment, the polypeptide includes at least 2 cysteine residues capable of forming disulfide bonds; in one such embodiment, the polypeptide includes at least one disulfide bond. In a further embodiment, each disulfide bond binds a first cysteine residue present in a first secondary structure domain to a second cysteine residue present in a second secondary structure domain.

In another embodiment, the polypeptide is at least 30% identical along its entire length to the amino acid sequence of any one of SEQ ID NOS: 1-333. In various further embodiments, the polypeptide is at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to the amino acid sequence of any one of SEQ ID NOS: 1-333, shown below, or mirror image thereof (i.e.: L amino acids substituted with D amino acids; D amino acids substituted with L amino acids). L amino acids and glycine are shown in upper case letters; D amino acids are shown in lower case letters. The secondary structure arrangement of each polypeptide is shown. “NC” means “non-canonical” (i.e.: either includes D-amino acids or is cyclic); “c” means that the peptide is cyclic, “mirror” means that the peptide is a mirror image of another peptide shown.

These designed peptides were screened against various protein databases, and are believed to share no more than 25% identity to any known peptide sequence.

NC_cHHH_D1
(SEQ ID NO: 01)
NPEDCRQDPEANKSPEECKKLK
NC_cHHH_D1_mirror
(SEQ ID NO: 02)
npedcrqdpeankspeeckklk
NC_cHH_D1
(SEQ ID NO: 03)
HDPEKRKECEKKYTDPKKREECKRKA
NC_cHH_D1_mirror
(SEQ ID NO: 04)
hdpekrkecekkytdpkkreeckrka
NC_cEE_D1
(SEQ ID NO: 05)
PVTWCVRIpPTVRCTVRp
NC_cEE_D1_mirror
(SEQ ID NO: 06)
pytwcyriPptyrctyrP
NC_cEE_D2
(SEQ ID NO: 07)
PVTWCVRIpPTVRCTVRd
NC_cEE_D2_mirror
(SEQ ID NO: 08)
pytwcyriPptyrctyrD
NC_cHLHR_D1
(SEQ ID NO: 09)
NPELQRKCKELdTRpeaerkcreeSD
NC_cHLHR_D1_mirror
(SEQ ID NO: 10)
npelqrkckelDtrPEAERKCREEsd
NC_EHE_D1
(SEQ ID NO: 11)
CQTWRrVSPEECRKYKEEYnCVRCTE
NC_EHE_D1_mirror
(SEQ ID NO: 12)
cqtwrRyspeecrkykeeyNcyrcte
NC_HEE_D1
(SEQ ID NO: 13)
NDKCKELKKRYPNCEVRCDpPRYEVHC
NC_HEE_D1_mirror
(SEQ ID NO: 14)
ndkckelkkrypncevrcdPpryevhc
NC_EEH_D2
(SEQ ID NO: 15)
TCVECapVKVCRPDPEEARREAEERC
NC_EEH_D2_mirror
(SEQ ID NO: 16)
tcvecAPvkvcrpdpeearreaeerc
NC_cHH_D2
(SEQ ID NO: 17)
PDPNRCEEYKRKVPNEDEVRKYCKKF
NC_cH_D2_mirror
(SEQ ID NO: 18)
pdpnrceeykrkvpnedevrkyckkf
NC_cHH_D3
(SEQ ID NO: 19)
PTDEKCEELKKRATDPEKRKELCKRA
NC_cHH_D3_mirror
(SEQ ID NO: 20)
PTDEKCEELKKRATDPEKRKELCKRA
NC_cHH_D3_mirror
(SEQ ID NO: 21)
ptdekceelkkratdpekrkelckra
NC_cHH32_D2
(SEQ ID NO: 22)
CDPRQKKTWTERARKSASEEEKKTWKDQCSKG
NC_cHH32_D5
(SEQ ID NO: 23)
ASPEYKKECEKRERDGDDPREISKCKTNAKRG
NC_cHH32_D39
(SEQ ID NO: 24)
QTEECKKKADEWKKKAEDPREHKKADELKKKC
NC_cHH32_D37
(SEQ ID NO: 25)
QSEECKKKADEWAKKAEDPREHETAKELKKKC
NC_cHH32_D30
(SEQ ID NO: 26)
QDPDCQSKAREKLKKAQNPEQKKDAKRIEKEC
NC_cHH32_D21
(SEQ ID NO: 27)
CSEEDEKKAKKLDKDGDDPRKAESLKRKCKKG
NC_cHH32_D26
(SEQ ID NO: 28)
SDPEEQKDLKRLIKECTDPDCRKDLKRKIKET
NC_cHH32_D28
(SEQ ID NO: 29)
QDPTCQKQADEWAKKAQDPNQKKHYKKLKETC
NC_cHH32_D13
(SEQ ID NO: 30)
ASEEWKDRCDKWKKSGADPSIQKECDEKIKKG
NC_cHH32_D14
(SEQ ID NO: 31)
ASPEECSKYRKLIKDGASEEEQKKFKKYCKDG
NC_cHH32_D31
(SEQ ID NO: 32)
PNPEKCSKAEELKRKYPDPTVQKKADELCKKD
NC_cHH32_D36
(SEQ ID NO: 33)
SDPDQHKKADELKKKCQTPECKTKADEWKKKA
NC_cHH32_D38
(SEQ ID NO: 34)
QSEECKKKADEWAKKAEDPTEHEQAKELKKKC
NC_cHH32_D4
(SEQ ID NO: 35)
ASPEICKKAEEAEKKNDDPRKIKELQEKCKKG
NC_cHH32_D3
(SEQ ID NO: 36)
CSEEDKKKAKTWKDQGADPTIQKKADDKCSKG
NC_cHH32_D15
(SEQ ID NO: 37)
CSDEQRKTAEELEKKGDDPTKIKKAKDTCSKG
NC_cHH32_D12
(SEQ ID NO: 38)
CSEEDKKRLEEARKKGADPTEIKKLTEKCQKG
NC_cHH32_D29
(SEQ ID NO: 39)
SDKECRDRLKKLIKDIPDPEARKELEKRAREC
NC_cHH32_D27
(SEQ ID NO: 40)
QDPRAKETAKEWKKKCQTEECQKRADKYAKDH
NC_cHH32_D20
(SEQ ID NO: 41)
ASEEICKKAEEAKKKGDDPKKIKTLDELCKKG
NC_cHH32_D11
(SEQ ID NO: 42)
DDPTVCKQAEEAKKKGDDPRKIKTLDTRCKQG
NC_cHH32_D16
(SEQ ID NO: 43)
ADPEQCKTWEKQAKEGADPSQQKDWKRKCKEG
NC_cHH32_D18
(SEQ ID NO: 44)
SSEEVCKSAEEAKKKGDDEKKAKDLDKECKDG
NC_cHH32_D23
(SEQ ID NO: 45)
ASPEECSKYRKLIKDGASEEEQKKYKKACKDG
NC_cHH32_D24
(SEQ ID NO: 46)
ADPTQCKRWKEEAKKGADPSQQETWEKQCKSG
NC_cHH32_D35
(SEQ ID NO: 47)
KDPKEQKKAKEQYKKCQTKECKDKAKERLDKA
NC_cHH32_D32
(SEQ ID NO: 48)
QSEECKKKADEWKKKAEDPEERKKAEELKQKC
NC_cHH32_D40
(SEQ ID NO: 49)
SDPECQKTLDTLIKQIPDPETQKDLKKKKKEC
NC_cHH32_D9
(SEQ ID NO: 50)
SDPSDCKTAEELKRKGDDPEKIKHYETLCKRG
NC_cHH32_D7
(SEQ ID NO: 51)
GSEEDCKTAEKLKKDGADPREIKTADEKCKKG
NC_cHH32_D25
(SEQ ID NO: 52)
QSEECKKKADTWKKQAQNPEERKKYDELKKKC
NC_cHH32_D22
(SEQ ID NO: 53)
DDPSVCKSAEKAKKKGDNPEKIKTLETRCKQG
NC_cHH32_D19
(SEQ ID NO: 54)
ASEEECDTARQLKEKGDDPTKIKHYDRRCKEG
NC_cHH32_D17
(SEQ ID NO: 55)
ASEEYKKTCEKKKKDGASEEEKKTCDENIKKG
NC_cHH32_D10
(SEQ ID NO: 56)
CSEEDKKKLEEARRKGDDPTNIKRLEDKCKKG
NC_cHH32_D6
(SEQ ID NO: 57)
ADPSVCKKAEEAKKKGDDPRRIKTWDELCKKG
NC_cHH32_D1
(SEQ ID NO: 58)
ASPEICTKAEEAEKKGDDPRKIKELQDKCKKG
NC_cHH32_D8
(SEQ ID NO: 59)
CSEEDKKTAETLKRQGADPTEQKKMDDKCSKG
NC_cHH32_D33
(SEQ ID NO: 60)
SDPETQKKLEEKAQKCSDPECRKTLKKLIKDT
NC_cHH32_D34
(SEQ ID NO: 61)
SDEDCQKTLDKLKKDVPDPNQQKEYDERKKKC
NC_cHH32_D2_mirror
(SEQ ID NO: 62)
cdprqkktwterarksaseeekktwkdqcskg
NC_cHH32_D5_mirror
(SEQ ID NO: 63)
aspeykkecekrerdgddpreiskcktnakrg
NC_cHH32_D39_mirror
(SEQ ID NO: 64)
qteeckkkadewkkkaedprehkkadelkkkc
NC_cHH32_D37_mirror
(SEQ ID NO: 65)
qseeckkkadewakkaedprehetakelkkkc
NC_cHH32_D30_mirror
(SEQ ID NO: 66)
qdpdcqskareklkkaqnpeqkkdakriekec
NC_cHH32_D21_mirror
(SEQ ID NO: 67)
cseedekkakkldkdgddprkaeslkrkckkg
NC_cHH32_D26_mirror
(SEQ ID NO: 68)
sdpeeqkdlkrlikectdpdcrkdlkrkiket
NC_cHH32_D28_mirror
(SEQ ID NO: 69)
qdptcqkqadewakkaqdpnqkkhykklketc
NC_cHH32_D13_mirror
(SEQ ID NO: 70)
aseewkdrcdkwkksgadpsiqkecdekikkg
NC_cHH32_D14_mirror
(SEQ ID NO: 71)
aspeecskyrklikdgaseeeqkkfkkyckdg
NC_cHH32_D31_mirror
(SEQ ID NO: 72)
pnpekcskaeelkrkypdptvqkkadelckkd
NC_cHH32_D36_mirror
(SEQ ID NO: 73)
sdpdqhkkadelkkkcqtpecktkadewkkka
NC_cHH32_D38_mirror
(SEQ ID NO: 74)
qseeckkkadewakkaedpteheqakelkkkc
NC_cHH32_D4_mirror
(SEQ ID NO: 75)
aspeickkaeeaekknddprkikelqekckkg
NC_cHH32_D3_mirror
(SEQ ID NO: 76)
cseedkkkaktwkdqgadptiqkkaddkcskg
NC_cHH32_D15_mirror
(SEQ ID NO: 77)
csdeqrktaeelekkgddptkikkakdtcskg
NC_cHH32_D12_mirror
(SEQ ID NO: 78)
cseedkkrleearkkgadpteikkltekcqkg
NC_cHH32_D29_mirror
(SEQ ID NO: 79)
sdkecrdrlkklikdipdpearkelekrarec
NC_cHH32_D27_mirror
(SEQ ID NO: 80)
qdpraketakewkkkcqteecqkradkyakdh
NC_cHH32_D20_mirror
(SEQ ID NO: 81)
aseeickkaeeakkkgddpkkiktldelckkg
NC_cHH32_D11_mirror
(SEQ ID NO: 82)
ddptvckqaeeakkkgddprkiktldtrckqg
NC_cHH32_D16_mirror
(SEQ ID NO: 83)
adpeqcktwekqakegadpsqqkdwkrkckeg
NC_cHH32_D18_mirror
(SEQ ID NO: 84)
sseevcksaeeakkkgddekkakdldkeckdg
NC_cHH32_D23_mirror
(SEQ ID NO: 85)
aspeecskyrklikdgaseeeqkkykkackdg
NC_cHH32_D24_mirror
(SEQ ID NO: 86)
adptqckrwkeeakkgadpsqqetwekqcksg
NC_cHH32_D35_mirror
(SEQ ID NO: 87)
kdpkeqkkakeqykkcqtkeckdkakerldka
NC_cHH32_D32_mirror
(SEQ ID NO: 88)
qseeckkkadewkkkaedpeerkkaeelkqkc
NC_cHH32_D40_mirror
(SEQ ID NO: 89)
sdpecqktldtlikqipdpetqkdlkkkkkec
NC_cHH32_D9_mirror
(SEQ ID NO: 90)
sdpsdcktaeelkrkgddpekikhyetickrg
NC_cHH32_D7_mirror
(SEQ ID NO: 91)
gseedcktaeklkkdgadpreiktadekckkg
NC_cHH32_D25_mirror
(SEQ ID NO: 92)
qseeckkkadtwkkqaqnpeerkkydelkkkc
NC_cHH32_D22_mirror
(SEQ ID NO: 93)
ddpsvcksaekakkkgdnpekiktletrckqg
NC_cHH32_D19_mirror
(SEQ ID NO: 94)
aseeecdtarqlkekgddptkikhydrrckeg
NC_cHH32_D17_mirror
(SEQ ID NO: 95)
aseeykktcekkkkdgaseeekktcdenikkg
NC_cHH32_D10_mirror
(SEQ ID NO: 96)
cseedkkkleearrkgddptnikrledkckkg
NC_cHH32_D6_mirror
(SEQ ID NO: 97)
adpsvckkaeeakkkgddprriktwdelckkg
NC_cHH32_D1_mirror
(SEQ ID NO: 98)
aspeictkaeeaekkgddprkikelqdkckkg
NC_cHH32_D8_mirror
(SEQ ID NO: 99)
cseedkktaetlkrqgadpteqkkmddkcskg
NC_cHH32_D33_mirror
(SEQ ID NO: 100)
sdpetqkkleekaqkcsdpecrktlkklikdt
NC_cHH32_D34_mirror
(SEQ ID NO: 101)
sdedcqktldklkkdvpdpnqqkeyderkkkc
sEEH_D9
(SEQ ID NO: 102)
YTVCCNGICYTNDNKDEAEKVKKKIC
sEEH_D7
(SEQ ID NO: 103)
TCVECNGVKVCRPDPEEARRLAEEKC
sEEH_D18
(SEQ ID NO: 104)
CRVCENNFCVDASSCEEAQRILEKYK
sEEH_D16
(SEQ ID NO: 105)
TRCCINGYCVESDSTKEVEDKCKKYA
sEEH_D11
(SEQ ID NO: 106)
TTVCINGFCCTAPTPEEAKRCAKELS
sEEH_D6
(SEQ ID NO: 107)
VTVCINGYCCTAPTPDEAEECARRLS
sEEH_D1
(SEQ ID NO: 108)
ACVTYCHVTVCTKDPEEAKRKAKEIC
sEEH_D8
(SEQ ID NO: 109)
CEVTYCNITVRAESCEKAEKIARKLC
sEEH_D22
(SEQ ID NO: 110)
LCICVNGECICIPNPDEARKAEKKMR
sEEH_D10
(SEQ ID NO: 111)
ACVTVCGYTVCRPDPEEARRIAEELC
sEEH_D17
(SEQ ID NO: 112)
VKVCICGYCYTASTDEEAKQAKKEMC
sEEH_D19
(SEQ ID NO: 113)
CCLTFGGRTFCADDCEEAKKLAKKAG
sEEH_D21
(SEQ ID NO: 114)
YCITCGNETYCSDDPEDAKRLCKEAL
sEEH_D14
(SEQ ID NO: 115)
YCFTLKGCTVCAPNPEDAKTELKKCA
sEEH_D13
(SEQ ID NO: 116)
ACVCVNGVCVCASSPQEAEEIARKIR
sEEH_D2
(SEQ ID NO: 117)
VTERYGDCEIHCPTQDCADQYKEECK
sEEH_D5
(SEQ ID NO: 118)
CEVQIDDCRVPACTEDEAKELCKKGE
sEEH_D12
(SEQ ID NO: 119)
CEVTLNGCTYRASSCEEAKRYLEKYC
sEEH_D15
(SEQ ID NO: 120)
STVCCNGYCEEAHDEDEEREIRERCK
sEEH_D20
(SEQ ID NO: 121)
YCITCNNQTFCAPDPEKAKELCKRAL
sEEH_D4
(SEQ ID NO: 122)
TELRRGDLRCECSTDEECKRLSKEIC
sEEH_D3
(SEQ ID NO: 123)
CKVKCGPVEYQATSQDECNEWRKKYC
sHEE_D18
(SEQ ID NO: 124)
PPECEKYKKKYPNCQVTTDNGQCTFRC
sHEE_D16
(SEQ ID NO: 125)
SDECEKLKKKYPNCKVEDHNGECRVKC
sHEE_D11
(SEQ ID NO: 126)
EPQCEELKRRYPNCTVTKDGNTCKVDC
sHEE_D24
(SEQ ID NO: 127)
NPECEKYKKKYPNCDVKEKNGQCTFEC
sHEE_D23
(SEQ ID NO: 128)
PPQCEEYKKKYPNCEVRDHNGECRVHC
sHEE_D3
(SEQ ID NO: 129)
SEDCKELQKKFPECQVEEHNGDCQVRC
sHEE_D4
(SEQ ID NO: 130)
YEKQKELQKKFPDCEVRCKDGQCQVHC
sHEE_D22
(SEQ ID NO: 131)
TERCKEYKKRYPNCEVRSHGNTCKVQC
sHEE_D25
(SEQ ID NO: 132)
SDKCKELKKRYPNCEVRCDGNRYEVHC
sHEE_D10
(SEQ ID NO: 133)
PPECEKLKKKYPNCDVTCDNGDSQIQC
sHEE_D17
(SEQ ID NO: 134)
SDECKEYKDKYPNCKVTQKNGQCHVQC
sHEE_D19
(SEQ ID NO: 135)
TPECEKLKKKYPNCDVSEDNGDCQVRC
sHEE_D5
(SEQ ID NO: 136)
SDEQRQLEEKRPDCEVRCRGTTCELKC
sHEE_D2
(SEQ ID NO: 137)
YECERQLKEKYPDCEVRVQDTECRWRC
sHEE_D1
(SEQ ID NO: 138)
CPIAEELKKRFPNCKVECHGDEYRVHC
sHEE_D6
(SEQ ID NO: 139)
YEREKELQKRFPNCEVRCRSNQCQVNC
sHEE_D8
(SEQ ID NO: 140)
SDECEEYKRKYPNCTVEQKGNTCEYRC
sHEE_D28
(SEQ ID NO: 141)
NPRCEEYKKRYPNCEVRDDNGRCEYRC
sHEE_D26
(SEQ ID NO: 142)
QPECEKLKRKYPNCEVTQDGTQCKVRC
sHEE_D21
(SEQ ID NO: 143)
TERCKEYKKRYPTCRVEDDNGDCRVHC
sHEE_D14
(SEQ ID NO: 144)
SDTCEELKRRYKNCEVRCRGTEYEVRC
sHEE_D13
(SEQ ID NO: 145)
SDRCEEYKRRYPNCEVRDENGNCKVRC
sHEE_D9
(SEQ ID NO: 146)
TPQCEEYKKRYPNCEVEDDNGDCQVRC
sHEE_D7
(SEQ ID NO: 147)
SEKCKELKKKYPNCEVREDNGRCEVHC
sHEE_D12
(SEQ ID NO: 148)
NPECEKLKKKYPNCNVECDNGDTRIEC
sHEE_D15
(SEQ ID NO: 149)
GEKCKEYKKKYPNCRVEERNGDCQVTC
sHEE_D20
(SEQ ID NO: 150)
SQECEDYKEKYRNCQISEDNGQCTFQC
sHEE_D27
(SEQ ID NO: 151)
DEDCEELKRRYKSCDVTKSGGQCKVDC
sHEE_D29
(SEQ ID NO: 152)
NPRCEEYKRRWPNCEVREHNGQCTYRC
NC_sEEH_D9_mirror
(SEQ ID NO: 153)
ytvccnGicytndnkdeaekykkkic
NC_sEEH_D7_mirror
(SEQ ID NO: 154)
tcvecnGykvcrpdpeearrlaeekc
NC_sEEH_D18_mirror
(SEQ ID NO: 155)
crycennfcvdassceeaqrilekyk
NC_sEEH_D16_mirror
(SEQ ID NO: 156)
trccinGycvesdstkevedkckkya
NC_sEEH_D11_mirror
(SEQ ID NO: 157)
ttycinGfcctaptpeeakrcakels
NC_sEEH_D6_mirror
(SEQ ID NO: 158)
vtvcinGycctaptpdeaeecarrls
NC_sEEH_D1_mirror
(SEQ ID NO: 159)
acytychytvctkdpeeakrkakeic
NC_sEEH_D8_mirror
(SEQ ID NO: 160)
ceytycnityraescekaekiarklc
NC_sEEH_D22_mirror
(SEQ ID NO: 161)
lcicvnGecicipnpdearkaekkmr
NC_sEEH_D10_mirror
(SEQ ID NO: 162)
acytycGytvcrpdpeearriaeelc
NC_sEEH_D17_mirror
(SEQ ID NO: 163)
ykycicGycytastdeeakqakkemc
NC_sEEH_D19_mirror
(SEQ ID NO: 164)
ccltfGGrtfcaddceeakklakkaG
NC_sEEH_D21_mirror
(SEQ ID NO: 165)
ycitcGnetycsddpedakrlckeal
NC_sEEH_D14_mirror
(SEQ ID NO: 166)
ycftlkGctvcapnpedaktelkkca
NC_sEEH_D13_mirror
(SEQ ID NO: 167)
acycvnGycycasspqeaeeiarkir
NC_sEEH_D2_mirror
(SEQ ID NO: 168)
vteryGdceihcptqdcadqykeeck
NC_sEEH_D5_mirror
(SEQ ID NO: 169)
cevqiddcrypactedeakelckkGe
NC_sEEH_D12_mirror
(SEQ ID NO: 170)
cevtlnGctyrassceeakrylekyc
NC_sEEH_D15_mirror
(SEQ ID NO: 171)
stvccnGyceeandedeereirerck
NC_sEEH_D20_mirror
(SEQ ID NO: 172)
ycitcnnqtfcapdpekakelckral
NC_sEEH_D4_mirror
(SEQ ID NO: 173)
telrrGdlrcecstdeeckrlskeic
NC_sEEH_D3_mirror
(SEQ ID NO: 174)
ckykcGpveyqatsqdecnewrkkyc
NC_sHEE_D18_mirror
(SEQ ID NO: 175)
ppecekykkkypncqyttdnGqctfrc
NC_sHEE_D16_mirror
(SEQ ID NO: 176)
sdeceklkkkypnckvedhnGecrykc
NC_sHEE_D11_mirror
(SEQ ID NO: 177)
epqceelkrrypnctytkdGntckvdc
NC_sHEE_D24_mirror
(SEQ ID NO: 178)
npecekykkkypncdykeknGqctfec
NC_sHEE_D23_mirror
(SEQ ID NO: 179)
ppqceeykkkypncevrdhnGecrvhc
NC_sHEE_D3_mirror
(SEQ ID NO: 180)
sedckelqkkfpecqyeehnGdcqvrc
NC_sHEE_D4_mirror
(SEQ ID NO: 181)
yekqkelqkkfpdcevrckdGqcqvhc
NC_sHEE_D22_mirror
(SEQ ID NO: 182)
terckeykkrypncevrshGntckvqc
NC_sHEE_D25_mirror
(SEQ ID NO: 183)
sdkckelkkrypncevrcdGnryevhc
NC_sHEE_D10_mirror
(SEQ ID NO: 184)
ppeceklkkkypncdvtcdnGdsqiqc
NC_sHEE_D17_mirror
(SEQ ID NO: 185)
sdeckeykdkypnckvtqknGqchvqc
NC_sHEE_D19_mirror
(SEQ ID NO: 186)
tpeceklkkkypncdvsednGdcqvrc
NC_sHEE_D5_mirror
(SEQ ID NO: 187)
sdeqrqleekrpdcevrcrGttcelkc
NC_sHEE_D2_mirror
(SEQ ID NO: 188)
yecerqlkekypdcevrvqdtecrwrc
NC_sHEE_D1_mirror
(SEQ ID NO: 189)
cpiaeelkkrfpnckvechGdeyrvhc
NC_sHEE_D6_mirror
(SEQ ID NO: 190)
yerekelqkrfpncevrcrsnqcqvnc
NC_sHEE_D8_mirror
(SEQ ID NO: 191)
sdeceeykrkypnctveqkGntceyrc
NC_sHEE_D28_mirror
(SEQ ID NO: 192)
nprceeykkrypncevrddnGrceyrc
NC_sHEE_D26_mirror
(SEQ ID NO: 193)
qpeceklkrkypncevtqdGtqckvrc
NC_sHEE_D21_mirror
(SEQ ID NO: 194)
terckeykkryptcrveddnGdcrvhc
NC_sHEE_D14_mirror
(SEQ ID NO: 195)
sdtceelkrrykncevrcrGteyevrc
NC_sHEE_D13_mirror
(SEQ ID NO: 196)
sdrceeykrrypncevrdenGnckvrc
NC_sHEE_D9_mirror
(SEQ ID NO: 197)
tpqceeykkrypnceveddnGdcqvrc
NC_sHEE_D7_mirror
(SEQ ID NO: 198)
sekckelkkkypncevrednGrcevhc
NC_sHEE_D12_mirror
(SEQ ID NO: 199)
npeceklkkkypncnvecdnGdtriec
NC_sHEE_D15_mirror
(SEQ ID NO: 200)
GekckeykkkypncrveernGdcqvtc
NC_sHEE_D20_mirror
(SEQ ID NO: 201)
sqecedykekyrncqisednGqctfqc
NC_sHEE_D27_mirror
(SEQ ID NO: 202)
dedceelkrrykscdvtksGGqckvdc
NC_sHEE_D29_mirror
(SEQ ID NO: 203)
nprceeykrrwpncevrehnGqctyrc
EEHE_1.3_04
(SEQ ID NO: 204)
CRFRAECQGNNVHVRGDGCKKEEIEKAWKKAEEWCKNGMQSSEREE
EEEH_3.0_08
(SEQ ID NO: 205)
CCKQQNENCYFAERTNKTFCYQDSKEQAREDCEEECRRS
EEEH_3.0_06
(SEQ ID NO: 206)
CSDCETECYCFVSKGKQWHGTSEECKKYKEEAEREC
HEEE_2.1_01
(SEQ ID NO: 207)
SCEEEAKKEADKCRKNGCQYRVDSDNCEVECRNCNIRKQF
EEHE_2.0_04
(SEQ ID NO: 208)
DCFFVIGGQDDQQCHTHQEECRKECEEKAEEQNRQCFDHCT
EEHE_2.0_03
(SEQ ID NO: 209)
KCYVICGNHDDYEFDTTREEECRRECEKARQEQNHECNCHYS
EEEH_3.0_01
(SEQ ID NO: 210)
EQYHCHGNYVRYICEDGQDCEYHADCSDEEAEREAKEECERQC
HEEE_2.1_06
(SEQ ID NO: 211)
KPEEYCRKVKDECKKRGLTRCHVTAKYGCECEVRGDTYQLRC
HHH_2.0_05
(SEQ ID NO: 212)
ECEKKAEECKRYAEEQNTSEECAERAEEYARRHCESSEEECREYAEECKKN
gHHH_06
(SEQ ID NO: 213)
PCEDLKERLKKLGMSEECRQRLEKMCKEGTSEDAERMARNCES
HEEE_2.2_05
(SEQ ID NO: 214)
TCQERVKEIKERCKKRGQEIRERPGDHEVQCGTERYRC
EHE_1.0_12
(SEQ ID NO: 215)
TCETYHVKRPDCREAEEEARKLRQECKDRGQCCTVTWTCK
HHH_2.0_02
(SEQ ID NO: 216)
PCQECERELEEAKRNNQCREERAEEIRREREEGQTSCEECKREAERCRQE
HHH_3.0_03
(SEQ ID NO: 217)
SECSKEACKQAETGTCDQFDEWLKRQGCPPTEDLDECRKRCKEN
EEH_1.0_11
(SEQ ID NO: 218)
CHITITCTHGTETRTETVKTTDPNECEKREKEIKNRC
HH_2.0_29
(SEQ ID NO: 219)
AQCEKDLKKVKKTGDPEKLDKIRKKCA
HHH_3.0_04
(SEQ ID NO: 220)
PCWKELKKSAEKRGNEKCKKLAEECHRRNLSCDECEKLYRKCS
EEH_1.0_07
(SEQ ID NO: 221)
CEKFKCNGQTYKYCDPNEAKKAKKKC
EEEH_4.0_01
(SEQ ID NO: 222)
NCQINGDTCQIGNEQCQNQEECKRLCEECEKS
EEEH_3.2_01
(SEQ ID NO: 223)
CVQRHPGKKVRCGNREEYQCTTDECVREMEEKCEKRC
EEHE_2.2_03
(SEQ ID NO: 224)
CVRCRHGNEERTYCCTSEECKREVKEKCDNDSTSRFHTG
EHE_1.0_03
(SEQ ID NO: 225)
KTCEFTIPNCSEEEARRYSKKKGCDETRWQCG
EEHE_2.2_04
(SEQ ID NO: 226)
DCEIRSQCSHVRTDDPNECERICKECKKRGYEVHCDNR
HH_2.0_36
(SEQ ID NO: 227)
ADCDKKLKKVQEKSKKGLTETVRKLKEKVEKC
EHE_1.0_04
(SEQ ID NO: 228)
QCVRFEFRPNDEEKKRKAEKACRELKKEGKCCEEKEG
EEH_1.0_09
(SEQ ID NO: 229)
TCIKYTNPNCGRTVERCGQDPEKIKKEASKC
EEEH_3.2_06
(SEQ ID NO: 230)
CRIEVRGTEVRCCDGTRCERYEMTSKEEAKKMEKKCRKKC
EHEE_1.7_04
(SEQ ID NO: 231)
DREERRCRGGKEEECRREAEKRCKEHNGTCEVRKQGNEIRIEIRR
HHH_4.0_03
(SEQ ID NO: 232)
CKEEMEKVCKEIGTEEKCKRIRKVAERGNCEEAQREAKRMKS
EEEH_3.0_10
(SEQ ID NO: 33)
CQEDIDGSHYRCFIRQTGSHCQCTTEECAKECDRQCEEEC
EHEE_1.7_03
(SEQ ID NO: 234)
NRDRRCYSSGRAEEIARRLAEEARRKGKTYEERKTGGTICVEIDE
HHH_4.0_04
(SEQ ID NO: 235)
SDDKAEQCCKEIGNEEKCRRLKEVAKDGSEEEVDEMCRRMRS
HHH_3.0_05
(SEQ ID NO: 236)
SSECEKKICKEWKKGTSEDELRKLCSSCTNNDKECDEAIKKCKK
gEEEH_04
(SEQ ID NO: 237)
CRCHITSSCVRVEGDNGEEYRYCSSDEEDLRRFCKEMQKQC
HHH_3.0_02
(SEQ ID NO: 238)
TSCEEEIKKLCKSGKRDPEEEKKVEKICRKCGVSEDQCEELKKKFRKC
EEH_1.0_10
(SEQ ID NO: 239)
CTTFRFTSPCGNTEVRVTTCDPNEKKEAQKEAEKLKKKCKKS
HEEE_2.2_04
(SEQ ID NO: 240)
SEECAERLREECERRNIPYEVRKTSTCITVQCGTERYTCC
HHH_2.0_03
(SEQ ID NO: 241)
KCEEAEREARECQENNQCREEELEKIEEKREKGETSCEEAKEEIERCCQS
HEEE_2.2_03
(SEQ ID NO: 242)
NPEDCARKVEEHCQRQGVRYTTHRQPTCIEVRCEKTTIRCC
HH_2.0_26
(SEQ ID NO: 243)
ADDIKKCEKKVRKDSNPDVKKKLKKCKKA
HHH_2.0_04
(SEQ ID NO: 244)
KCWRKAKEECRKAQEGKTQEEECKEACRECKERGESSEEECKEAEKEARKE
EEHE_2.0_02
(SEQ ID NO: 245)
ECYFFIGGTDDQECQSEQEECRKKAEEKCREQNQQCVDDCK
EEEH_3.0_07
(SEQ ID NO: 246)
TCDCKDHETIFCNCPGNDDDQASTREECKKKCEERES
gEHEE_06
(SEQ ID NO: 247)
EERRYKRCGQDEERVRRECKERGERQNCQYQIRKEGNCYVCEIRC
EEHE_2.0_05
(SEQ ID NO: 248)
CIVICDCETDDDDDQQNCREEEAREEARKREEECGEQFTCHVQT
EEE_EEE_1.1_06
(SEQ ID NO: 249)
PVECRRTSKHVEVRCGNVQVRTSEDCQCSEKNNRVHIQCSKTREEYQC
EEEH_3.0_09
(SEQ ID NO: 250)
CCREEYQNHEWFVEHPEPRRFRCDNTRCEEAEERCDEECRK
EEE_EEE_1.1_01
(SEQ ID NO: 251)
VCRIEWTTTSCRIDCGTEEYHVEPGKEICVGNFCVRVTNTTCTVQSN
EEEH_1.4_03
(SEQ ID NO: 252)
KECRIRHRGDKARVRVRDGGTSEEREVKCDGDDNKCKEAYQRICEEWERKR
EEEH_1.4_12
(SEQ ID NO: 253)
CQMREETRGNTIVMRVQGGRDSEEFRKKGGAREEEERKYRKKAEDKCKNNQ
EEHE_2.1_06
(SEQ ID NO: 254)
TCNVTCDNRDTQTFDDCEECKKKAKECKSEGRDVQIQCG
EHEE_1.7_02
(SEQ ID NO: 255)
ECRTYRQKGKREEECRRLCEEIRKRENGTVDCQIDGNECEIRACR
HHH_4.0_05
(SEQ ID NO: 256)
SCDECYKKMQKTGPPNTEKVKELWKRCQKDESSEYCRRMKKMAK
gEEH_04
(SEQ ID NO: 257)
QCYTFRSECTNKEFTVCRPNPEEVEKEARRTKEEECRK
EHEE_1.7_05
(SEQ ID NO: 258)
QRTRKECDSNNMDECEKRCREEARRKNCRVEIRTRGNKVYCRFEC
HHH_4.0_02
(SEQ ID NO: 259)
CEDELRELCKRVGDPKCCEEMKKMLKTGTCDEARKMLEKCLK
EEHE_2.1_01
(SEQ ID NO: 260)
CCEVTSRSGESRTFCGASRDECEKEAQRCEKEAGVECRWEDK
EEHE_2.2_05
(SEQ ID NO: 261)
TCHVRCGNITEQTFTTGTCDEMCRKMEEECRKLGGQVDCTSL
EHE_1.0_05
(SEQ ID NO: 262)
CKYTFQFCNYDTEQAKEECRKAEEKVKKTHPECEVQCQEC
gHEEE_02
(SEQ ID NO: 263)
SQETRKKCTEMKKKFKNCEVRCDESNHCVEVRCSDTKYTLC
EEH_1.0_08
(SEQ ID NO: 264)
TIKIDCNGEEYKCEDPNRCEEIKRKC
gEEHE_02
(SEQ ID NO: 265)
PCECDVNGETYTVSSSEECERLCRKLGVTNCRVHCG
EHE_1.0_02
(SEQ ID NO: 266)
TCSVTVTGSRSQCEEVQRQLKKKGQPCQVECDN
EEH_1.0_01
(SEQ ID NO: 267)
CQTWTFPGCNQTVTECTDEDHKKAREVEKKCG
EEH_1.0_06
(SEQ ID NO: 268)
TYCLTVEFTCPRGERYEETFCSDTPEEAKKERKKFETEAEKKCRG
HH_2.0_45
(SEQ ID NO: 269)
CDDVKKEVEEIKKKLTSEDLKKVQEKLDKC
HEEE_3.0_01
(SEQ ID NO: 270)
CEECKEMARECKEKNQDNCEKTDSQCTYKDNQVKCQS
gEEE_EEE_02
(SEQ ID NO: 271)
TCEIRVTDTHCKVHCGTQEYKVPPGRTLKVGNCRFTYHDTTCTVECR
HHH_4.0_08
(SEQ ID NO: 272)
DCERIRKTVKDLGCSDEMKEKAERCCRGEYNPEECDRELKKCK
HH_2.0_01
(SEQ ID NO: 273)
ADDCKKVQKKVKELNKTNSDDSLKEVKKLQKKCA
EEHE_2.0_10
(SEQ ID NO: 274)
CVICICGNQEQQTSNTHEKECKEEAEEAERQGCDCKVTT
HHH_4.0_01
(SEQ ID NO: 275)
KCEDLRKECRKVGGNPEYEKRIEKMCRDGNDEEAERVARKCKS
EEHE_2.1_02
(SEQ ID NO: 276)
TCEVRCENGQRIEYPATSDEECERWCRKAKKEFPNYRCTCTHK
EEHE_2.1_05
(SEQ ID NO: 277)
GCEIRCGNGYTWTVSDNEEKCKRECEKAKKSGCQDVNCTRR
EEEH_3.2_03
(SEQ ID NO: 278)
CVEKRGSRVHCKAHNKEFQCPPTPDEIERCREECEKRC
EEHE_2.2_01
(SEQ ID NO: 279)
RCTVELCGRRYECRTDESQLENCAREMQRRVGCPQKPRLECR
EHE_1.0_01
(SEQ ID NO: 280)
TCSVTVNTGTPDEDKKECKRVQEEAERKGTQCQCQQE
HH_2.0_34
(SEQ ID NO: 281)
ADDIEKCRKKVEKNSSSQDVQEQLRKCKEA
HH_2.0_48
(SEQ ID NO: 282)
CAQELEDRVRKLEKKLRKKNDDTQVEKLQKKLDELKKRAVC
EHE_1.0_08
(SEQ ID NO: 283)
CSYTVRFCYTTEEERKEREERVKKNCKRSGCECRWTNERC
EEEH_4.0_04
(SEQ ID NO: 284)
CDFNQHGNNMTCNGENDTHCNNDEECKKECEKMKENC
EEH_1.0_05
(SEQ ID NO: 285)
TTCVTRRNDDCGQEVTVCSDSEEEARKRAEEILQRRCN
EEEH_4.0_03
(SEQ ID NO: 286)
CQKDDNGQDCRIDGKHQVECDNDEECCKEIEERACK
EEH_1.0_02
(SEQ ID NO: 287)
TCVTVESSCGRRVTVCRPNPEEAEREARKELKKEC
HHH_3.0_01
(SEQ ID NO: 288)
PCKEQAKKCYKERPKCNQEELERRVCEAEKRGLDEEEKKKLCNSCD
HHH_2.0_09
(SEQ ID NO: 289)
ECERAKEEAKKECSQGSSKEECRERCQEAAKDSDECVEKACQEAAE
HHH_3.0_06
(SEQ ID NO: 290)
NC_EKLKRKLEKACREGNCDKARKAYEEAQRQNCETDEIRKIYKECEKNC
HHH_2.0_07
(SEQ ID NO: 291)
CERCKKKLEECKGSSREDARERCEEAKQESCCSEEERREAEEEKQRA
EHE_1.0_10
(SEQ ID NO: 292)
CSTRVTVCNSNDEEAKKIKKRVCEEAKKRGCQCETETCRK
EEEH_3.0_04
(SEQ ID NO: 293)
EDIQCQSEGYIVVDCGQHQCKFDYDCSDEQQREEAREEAEKCC
HEEE_2.1_03
(SEQ ID NO: 294)
SEKTRKECEKQREKCGGRPCEYKGPNNCRCEIDGNTYSVDC
gHH_44
(SEQ ID NO: 295)
AEDCERIRKELEKNPNDEIKKKLEKCQA
EEHE_2.0_06
(SEQ ID NO: 296)
ECVVVCSDGQEQQRQDPCEQVCEEEQRKKGNHDCRCTQT
HHH_4.0_10
(SEQ ID NO: 297)
PCDRCARELEEAYPNNPEVNEEARRVKKNCTDEMCKEVKKMKKR
EEHE_2.0_01
(SEQ ID NO: 298)
DCCVICSGNDQYCAGDNNEEQAEREAKRCEEEGKQYHKYCH
EEEH_3.0_03
(SEQ ID NO: 299)
SEVRCDGNYCFVIACSGDEQSRDFRCDDEQEKEECKKEAEKEC
HEEE_2.1_04
(SEQ ID NO: 300)
SDENKKRCETEAKKCKKNGYRVECRNRGTCWEVDCEETTYTIC
EEE_EEE_1.1_05
(SEQ ID NO: 301)
TCEVRWTNTHCRIKCGTQEYECPPRRRCEIGNFHVDVHDTTCRLHSR
gEHE_06
(SEQ ID NO: 302)
CKQRRRYRGSEEECRKYAEELSRRTGCEVEVECET
EEHE_2.0_08
(SEQ ID NO: 303)
PCCIVYCETQFQHCADTKEKCERQCEEDERQDSQCRSRCTS
EEEH_4.0_02
(SEQ ID NO: 304)
SCHIDGNQCTYNNTDCNNREECKEYCEKCEKS
EEH_1.0_03
(SEQ ID NO: 305)
TCITTTCKGENETKTFCSDDEERIKKESKRCEG
EHE_1.0_09
(SEQ ID NO: 306)
TCSETYTFRGNPDECEKRHQELEREAREKGCQFQLECRN
HH_2.0_47
(SEQ ID NO: 307)
ADCDKKLKKVEERSKNGLTEEVQQLRDKVKKC
EHE_1.0_07
(SEQ ID NO: 308)
TCKKVTVEGNPDECQEVKKEARKEEEKKGTCVEVECKN
HH_2.0_35
(SEQ ID NO: 309)
ADDCKKLKEKLKKVKKNNGSDEIKKRVEKLRKKCEA
EEEH_3.2_05
(SEQ ID NO: 310)
RECRINNCREVRFRCPSGQTWTMTVTSCEEAKKMCEKMKKQC
EEEH_3.2_02
(SEQ ID NO: 311)
CRVECKPGGTCEVHRDSGKREEYTFPTSQDEVCKECKKLQKKC
HHH_2.0_10
(SEQ ID NO: 312)
QCERCCEAAKQKNREEAKEACERCQSGDTHEKDAEERCKEAET
EEHE_2.1_04
(SEQ ID NO: 313)
PCEINSDGCTRQEIPATSPEECKEACERAKKKCTSPVDCQHK
HHH_4.0_07
(SEQ ID NO: 314)
PCDEIEKKVRKRGCDPQVEKEVRRVCEEQNDSEQMKQIWKDCS
EEHE_2.1_03
(SEQ ID NO: 315)
ECTVRCGNQKYRCTTGTCDECAREIEEKCRKLGLEVEIRTL
EEHE_1.3_18
(SEQ ID NO: 316)
DEAECRIDGNECRLDAKGASDDAREECRELCEEACKKGQKRLQCKR
EHEE_1.7_09
(SEQ ID NO: 317)
QKETRHCSGQRCEQEARRWCEECKKKGKRVRCRKHGNQVEVQCDK
HHH_4.0_09
(SEQ ID NO: 318)
GCEDIDREVEKRGCTEDARRELQKLCKNGQTEDEIRRAADELC
EEE_EEE_1.1_04
(SEQ ID NO: 319)
QCEVRFTDTHCRVRCGTQEYKLEPGRRVRIGTSEFDVQPTTCTYSHI
EEHE_2.0_09
(SEQ ID NO: 320)
QCRVICQGHSTTEFSDDSKEECEKECERCEKDGYDSDCHQS
EEE_EEE_1.1_03
(SEQ ID NO: 321)
ESRCKKSSNTWFCEVGTVQVECPPGRRCTINNQYICEVQGNTCRTENE
HEEE_2.1_05
(SEQ ID NO: 322)
PCREEAKKRKEEAERKCTTLRVQCPSGCHFEIRCGNQIQEKC
EEEH_3.0_02
(SEQ ID NO: 323)
NCHEYHGECWYCFVDGDSQFHYHKCDKNAEEAKERKERCERDCS
HEEE_2.1_02
(SEQ ID NO: 324)
DERDKCAEEIRRECEERGLEVEIRKTDDCVRIRCGTEERTCC
EEEH_3.0_05
(SEQ ID NO: 325)
EEYRCHGNFVVFYCEQGQEYRCQADCSDEQERERCREEAEKQC
EEHE_2.0_07
(SEQ ID NO: 326)
ECIICCEGNQCRKFTQEEECKRQAKECEKQGLRYTTIDK
HEEE_2.2_06
(SEQ ID NO: 327)
SESEKMCRQCEEERKKYPTQETSVRLPKQNCECRVGSTTVDCDC
EHE_1.0_11
(SEQ ID NO: 328)
CRYEKETRGDDEQCRKEKEKLCEEAKKEEPRCQCHFRCQKG
HHH2.0_01
(SEQ ID NO: 329)
QCEEYARELREEAERQNCEEAREKAEECEEKNDCECAKEAEEKLRECS
HEEE_2.2_01
(SEQ ID NO: 330)
REEEVKKCCKEWHRRMKPDTFQVRTREGKCTVSRGRTYQC
HHH_2.0_06
(SEQ ID NO: 331)
EEERRCAEECCQQFSQKEECCERCEECANQQERAEKAKKDAC
HHH_2.0_08
(SEQ ID NO: 332)
ECYKEYCQEIKECQSTSEEEAEERAREACNTSCEEARKKAEEACQS
EEH_1.0_12
(SEQ ID NO: 333)
QCFEVEVNCPDKNQSFRYRFCSSNPEEAERRAREAEKRARENCK

The polypeptides described herein may be chemically synthesized or recombinantly expressed (when the polypeptide is genetically encodable). The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

As will be understood by those of skill in the art, the polypeptides of the invention may include additional residues at the N-terminus, C-terminus, or both that are not present in the polypeptides of the invention; these additional residues are not included in determining the percent identity of the polypeptides of the invention relative to the reference polypeptide.

As shown in the examples that follow, the specific primary amino acid sequence is not a critical determinant of maintaining the structure of the constrained peptide. Thus, the polypeptides of SEQ ID NO: 1-333 may be substituted with conservative or non-conservative substitutions. In one embodiment, changes from the reference polypeptide may be conservative amino acid substitutions. As used herein, “conservative amino acid substitution” means an amino acid substitution that does not alter or substantially alter polypeptide function or other characteristics. In one such embodiment, L amino acids are substituted with other L-amino acids, D amino acids are substituted with other L amino acids, and glycine may be substituted with L or D amino acids, preferably with D amino acids.

In other embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

As noted above, the polypeptides of the invention may include additional residues at the N-terminus, C-terminus, or both. Such residues may be any residues suitable for an intended use, including but not limited to detection tags (i.e.: fluorescent proteins, antibody epitope tags, etc.), linkers, ligands suitable for purposes of purification (His tags, etc.), and peptide domains that add functionality to the polypeptides.

In a further aspect, the present invention provides isolated nucleic acids encoding a polypeptide of the present invention that can be genetically encoded. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present invention provides recombinant expression vectors comprising the isolated nucleic acid of any aspect of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting host cells is well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In various embodiments, the expression vector may comprise a plasmid, viral-based vector, or any other suitable expression vector. In a further aspect, the present invention provides host cells that comprise the recombinant expression vectors disclosed herein, wherein the host cells can be either prokaryotic or eukaryotic. The cells can be transiently or stably engineered to incorporate the expression vector of the invention, using standard techniques in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium. Methods to recover polypeptide from cell free extracts or culture medium are well known to the person skilled in the art.

I. Accurate De Novo Design of Hyperstable Constrained Peptides

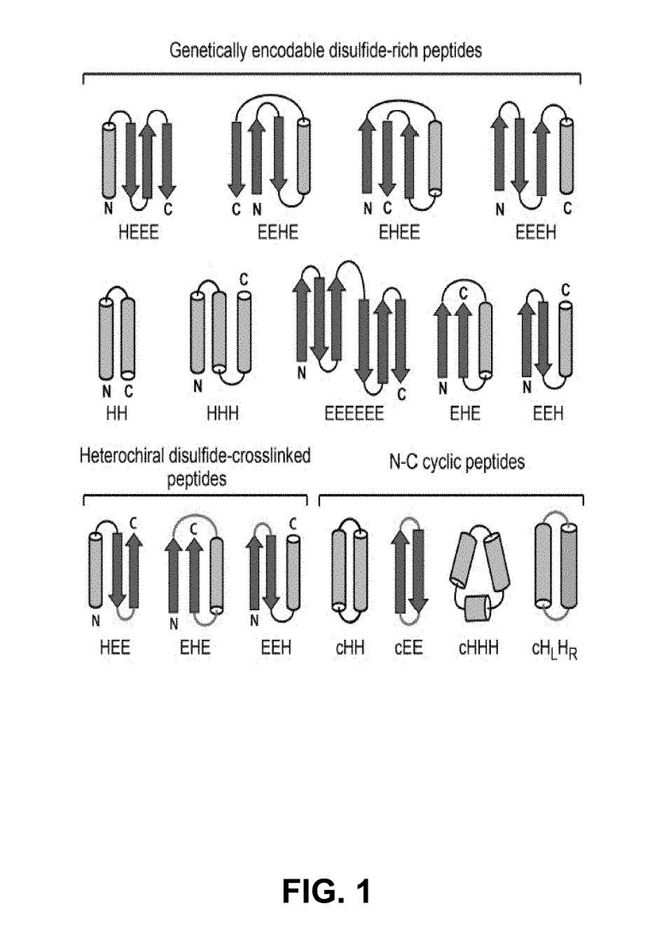

A structurally diverse array of 15-50 residue peptides has been designed spanning two broad categories: (i) genetically-encodable peptides, such as disulfide-rich peptides; and (ii) heterochiral peptides with non-canonical architectures and sequences. Genetic encodability has the advantage of being compatible with high-throughput selection methods, such as phage, ribosome, and yeast display, while incorporation of non-canonical components allows access to new types of structures, and can confer enhanced pharmacokinetic properties. To explore the folds accessible to genetically-encoded constrained peptides under 50 amino acids, nine topologies were selected: HH, HHH, EHE, EEH, HEEE, EHEE, EEHE, EEEH, and EEEEEE (FIG. 1; a “topology” is defined as the sequence of secondary structure elements in the folded peptide, where H denotes α-helix and E denotes β-strand). To explore the expanded design space accessible with inclusion of non-canonical amino acids and backbone cyclization, topologies containing two to three canonical secondary structure elements: HH, HHH, EEH, EHE, HEE, and EE, were sought, along with H_{L}H_{R}, a cyclic topology with right- and left-handed helices.

All of the design calculations described herein were carried out with the Rosetta™ software suite and followed the same basic approach. Large numbers of peptide backbones were stochastically generated as described in the following sections, combinatorial sequence design calculations were carried out to identify sequences (including disulfide crosslinks) stabilizing each backbone conformation, and the designed sequence-structure pairs were assessed by determining the energy gap between the designed structure and alternative structures found in large-scale structure prediction calculations based on the designed sequence. A subset of the designs in deep energy minima were then produced in the laboratory, and their stabilities and structures were determined experimentally.

Genetically-Encodable Disulfide-Constrained Peptides

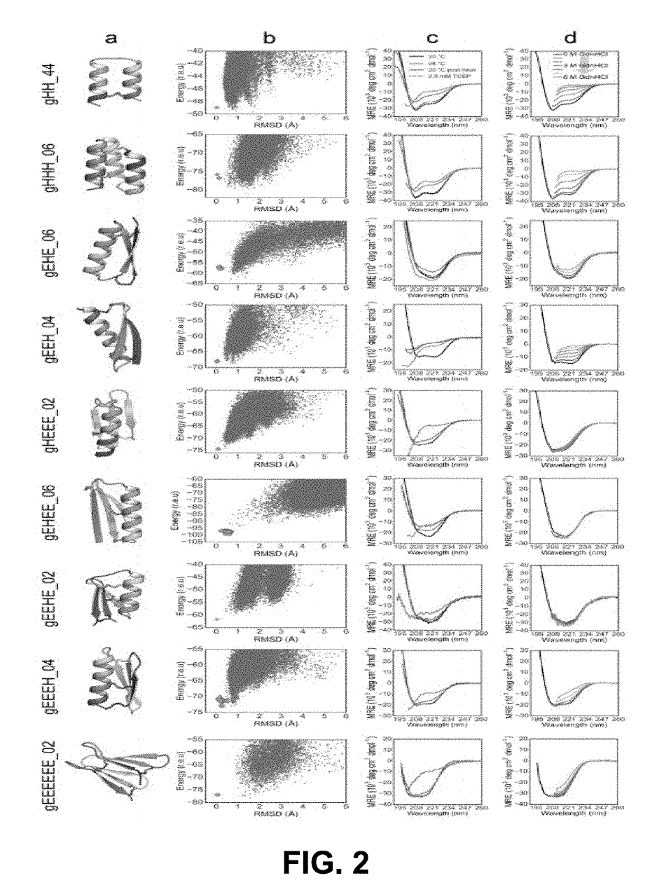

To design disulfide-stabilized genetically-encodable peptides, a “blueprint” was created specifying the lengths of each secondary structure and connecting loop for each topology. Ensembles of backbone conformations were generated for each blueprint by Monte Carlo-based assembly of short protein fragments, or, in the case of HH and HHH topologies, by varying the parameters in parametric generating equations. The backbones were scanned for sites capable of hosting disulfide bonds with near-ideal geometry and one to three disulfide bonds were incorporated. Low-energy amino acid sequences were designed for each disulfide-crosslinked backbone using iterative rounds of Monte Carlo-based combinatorial sequence optimization while allowing the backbone and disulfide linkages to relax in the Rosetta™ all-atom force field. Except for the EHEE topology, no manual amino acid sequence optimization was performed. Rosetta™ ab initio structure prediction calculations were carried for each designed sequence, and synthetic genes were obtained for a diverse set of 130 for which the target structure was in a deep global free energy minimum (FIG. 2 a,b).

Disulfide bonds in peptides are unlikely to form in the reducing environment of the cytoplasm, so designs were secreted from Escherichia coli or cultured mammalian cells. Twenty-nine designs exhibited a redox-sensitive gel-shift, redox-sensitive HPLC migration, and/or a CD spectrum consistent with the designed topology. All twenty-nine contain at least one non-alanine hydrophobic residue on each secondary structure element contributing van der Waals interactions in the core, which are likely important for proper peptide folding. One representative design from each topology for further biochemical characterization was chosen. Since eight of the nine topologies contained four or more cysteine residues, multiple-stage mass spectrometry to investigate the disulfide connectivity were used. In all cases the data were consistent with the designed connectivity.

The stability of the designs to thermal and chemical denaturation was assessed by CD spectroscopy. Samples were heated to 95° C. (FIG. 2c), or incubated with increasing concentrations of guanidinium hydrochloride (GdnHCl) (FIG. 2d). The contribution of disulfide bonds to protein folding was assessed by incubating samples with a ˜100-fold molar excess of the reductant tris (2-carboxyethyl) phosphine (TCEP). Designs gHEEE_02, gEEEH_04, and gEEEEEE_02 are resistant to both thermal and chemical denaturation, while design gHH_44 is resistant to thermal denaturation. gHEEE_02 contains three disulfide bonds, with each secondary structure element participating in at least one disulfide bond, and no two secondary structure elements sharing more than one disulfide bond. gEEEH_04 has two of three disulfide bonds linking the N-terminal β-strand to the C-terminal α-helix. gEEEEEE_02 consists of two antiparallel β-sheets packing against one another in a sandwich-like arrangement, with each β-sheet stabilized by a disulfide bond linking one terminus to its adjacent β-strand. gHH_44 consists of two α-helices with a single disulfide bond connecting the termini.

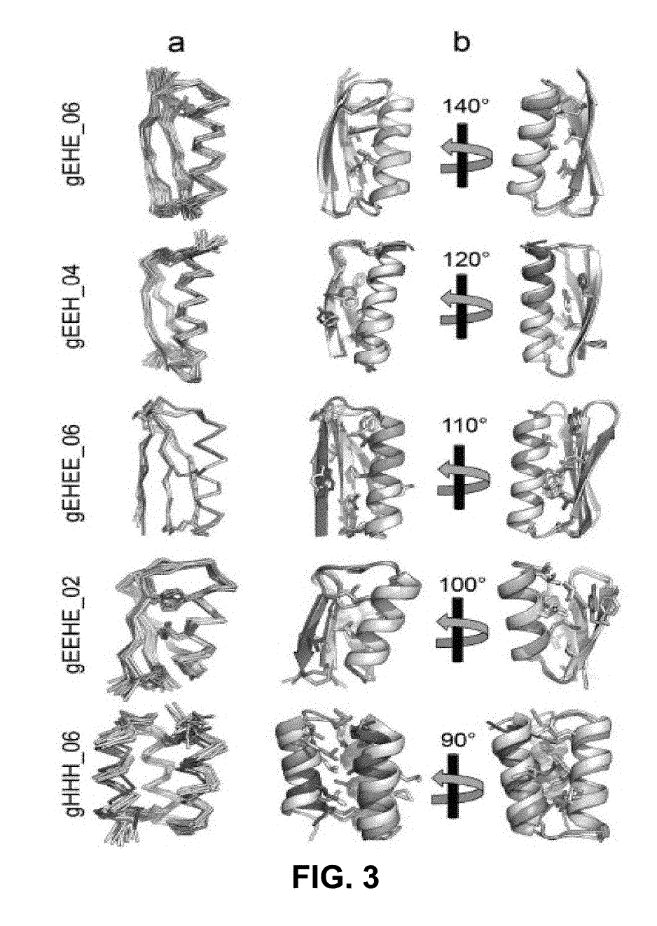

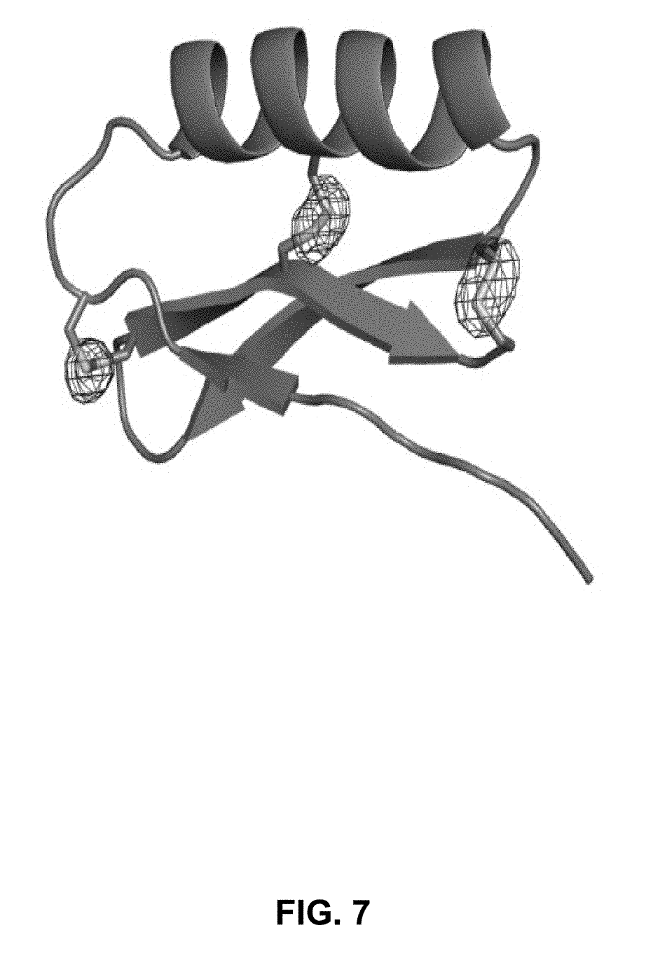

Design gEHEE_06 was crystallized and the structure determined to a resolution of 2.09 Å (FIG. 3, Table 2). The crystals had threefold non-crystallographic symmetry, and each protomer aligns to the design model with a mean all-atom RMSD of 1.12 Å. All three of the designed disulfide bonds were well-defined by electron density (FIG. 7), and rotamers of core residues exhibited excellent agreement with the design model. The protein was thermostable and completely resistant to chemical denaturation (FIG. 2c,d). While gEHEE_06 shares the short-chain scorpion toxin topology, the length of secondary structure elements and loops, and the position of the disulfide bonds, are entirely divergent from known natural peptides.

As crystallization efforts for other designs were unsuccessful (with phase-separation rather than protein precipitation observed), isotope-labelled peptides in E. coli were expressed and structures were determined by nuclear magnetic resonance (NMR) spectroscopy (see Experimental Methods). Upfield chemical shifts of the cysteine β-carbons (deposited in the BMRB) confirmed the formation of the designed disulfide bonds. Design gEEHE_02, with one disulfide bond connecting the termini within the β-sheet and two between the α-helix and β-sheet, aligns to the NMR ensemble with a mean all-atom RMSD of 1.44 Å. This design was impervious to both thermal and chemical denaturation (monitored by CD spectroscopy), and remained partially folded in the presence of TCEP. The final three designs are each composed of three secondary structure elements, with termini located at opposite ends of the molecule and two disulfide bonds connecting each terminus to the middle structural element or adjacent loop. gEEH_04 was less stable than the others to thermal denaturation, but its NMR structure is nearly identical to the design model (mean all-atom RMSD of 1.29 Å) gEHE_06, which contains a solvent-exposed two-strand parallel β-sheet (rare in natural protein structures), aligns to the NMR ensemble with an all-atom mean RMSD of 1.95 Å. It was thermally and chemically stable based on CD measurements, and remained folded in the presence of TCEP. gHHH_06 partially unfolds upon heating to 95° C. but returns to the folded state upon cooling; the design model aligns to the NMR ensemble with a mean all-atom RMSD of 1.74 Å. Taken together, the X-ray crystallographic and NMR structures demonstrate that this computational approach enables accurate design of protein mainchain conformation, disulfide bonds, and core residue rotamers.

Synthetic Heterochiral Disulfide-Constrained Peptides

Shorter disulfide-constrained peptides incorporating both L- and D-amino acids were also designed The Rosetta™ energy function was generalized to support D-amino acids by inverting the torsional potentials used for the equivalent L-amino acids (see Experimental Methods), and sequence design algorithms were extended to enable mixed-chirality design. Since chemical synthesis is labor-intensive, the development of automated computational screening techniques was prioritized, supplementing Rosetta™ ab initio screening with molecular dynamics (MD) evaluation.

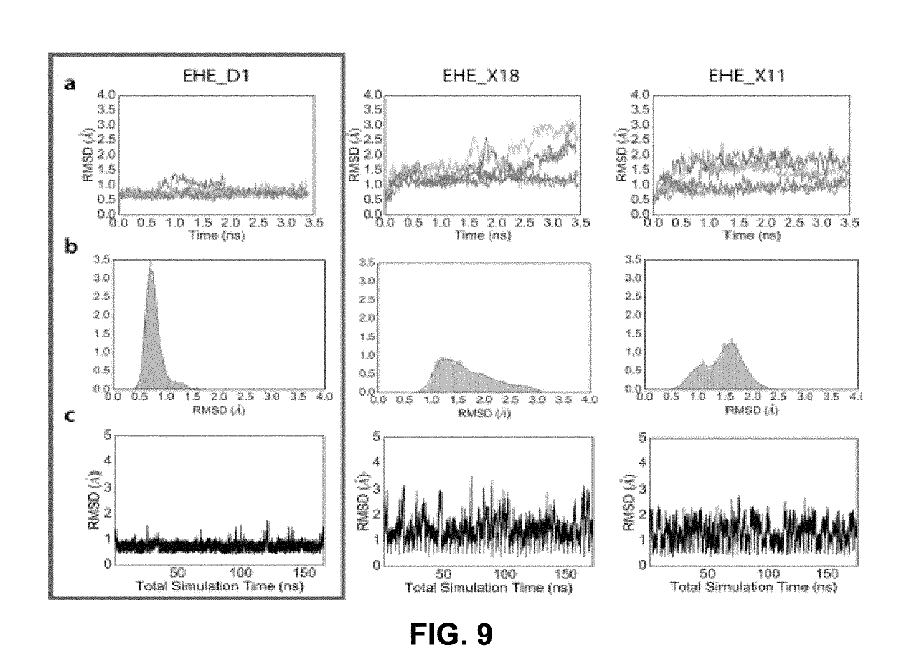

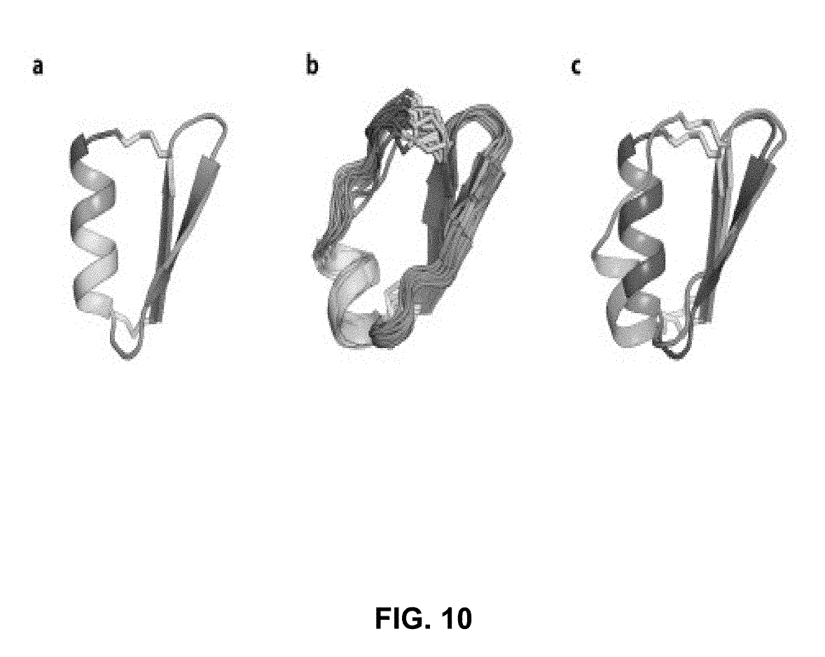

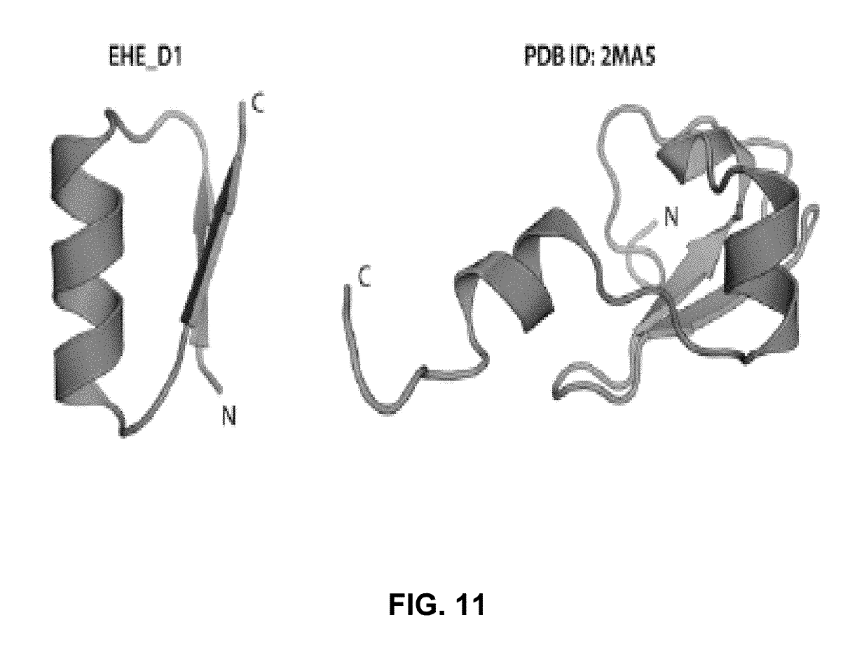

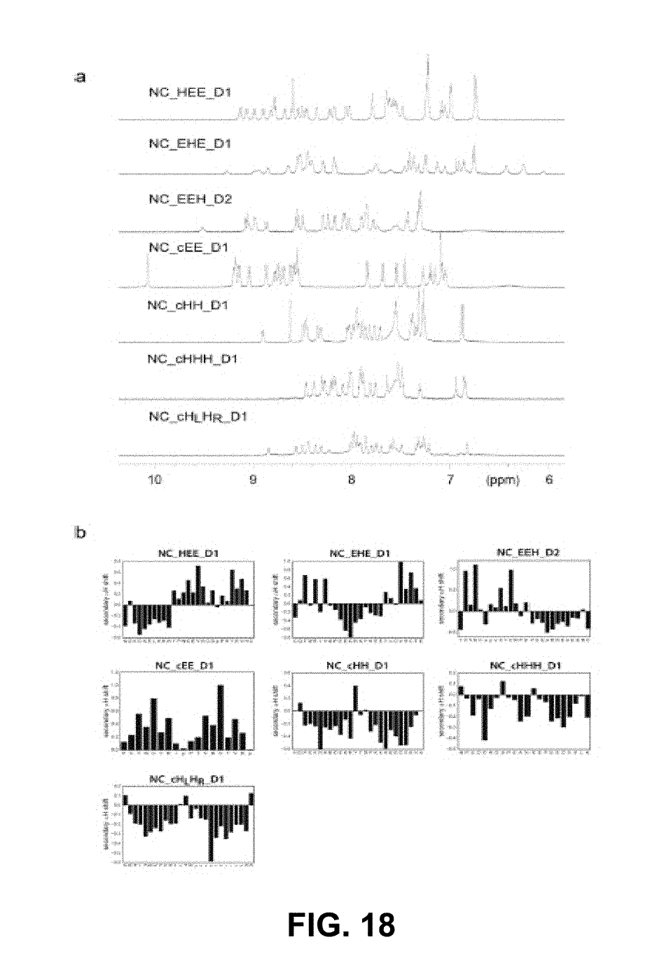

Large numbers of disulfide-constrained backbones for topologies HEE, EHE, and EEH were generated by fragment assembly as described above for genetically-encodable peptides. Sequences were designed (permitting D-amino acids at positive-phi positions), and the resultant low-energy designs were evaluated using MD and ab initio structure prediction (FIG. 9). For each topology, a single, low-energy design was selected (FIG. 10) which underwent only small (<1.0 Å RMSD) fluctuations in the MD simulations (FIG. 11) and had a significant energy gap in the structure prediction calculations. Selected peptides were chemically synthesized, and structurally characterized by NMR. In all three cases, the NMR spectra had well-dispersed, sharp peaks and secondary ¹H_{α} chemical shifts consistent with the secondary structure of the design model (FIG. 18).

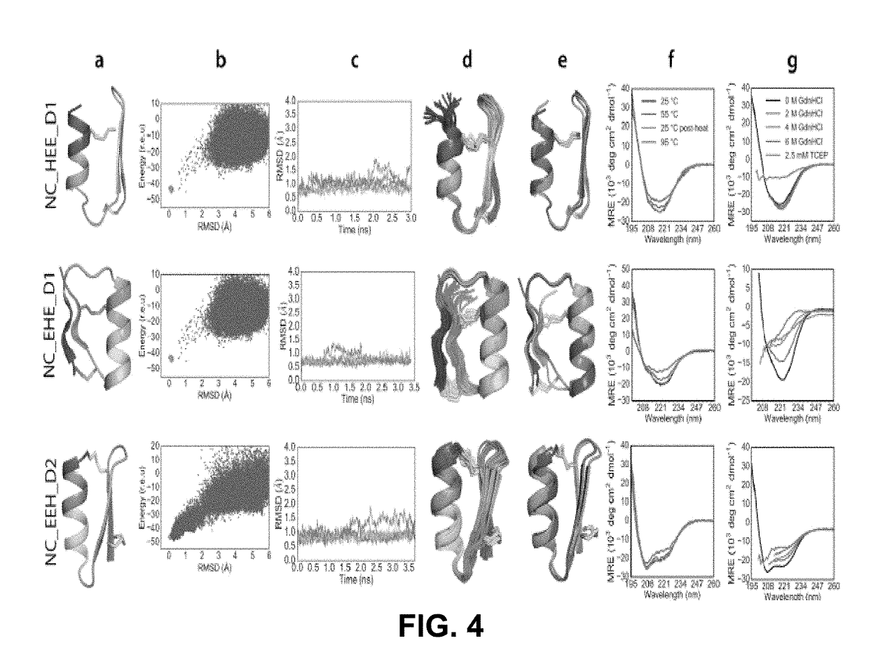

High-resolution NMR solution structures were determined for each of the designs (Table 3). NC_HEE_D1 is a 27-residue peptide with a D-proline, L-proline turn at the β-β junction; in this case, Rosetta™ re-identified a motif known previously to stabilize type II′ turns. The NMR structure closely matches the design model: the C_{α} RMSD is 0.99 Å between the designed structure and the lowest-energy NMR model (FIG. 4, top row). NC_EHE_D1 is a 26-residue peptide crosslinked using two disulfide bonds with a D-arginine residue in the β-a loop and a D-asparagine residue as the C-terminal capping residue for the α-helix. The design model has a 1.9 Å C_{α} RMSD to the lowest-energy NMR ensemble member, and 0.68 Å C_{α} RMSD to the closest member of the ensemble (FIG. 4, middle row; the last two residues at C-terminal vary considerably in the ensemble). NMR characterization of NC_EEH_D1 design showed an unwound C-terminal α-helix adopting an extended conformation, differing from the design model (FIG. 10). It was hypothesized that substantial strain was introduced by the angle between the helix and the preceding strand, and by the disulfide bonds at both ends of the helix. A second design for the same topology, NC_EEH_D2, has a type I′ turn at the β-β connection and a different disulfide pattern. The NMR ensemble for NC_EEH_D2 is very close to the design model (0.86 Å C_{α} RMSD to the lowest-energy NMR model; FIG. 4, bottom row).

The stability of the designed peptides was explored using CD spectroscopy to monitor thermal and chemical denaturation. All three peptides are very thermostable: there is no loss in secondary structure for NC_HEE_D1 and NC_EEH_D2 at 95° C., and only a small decrease for NC_EHE_D1 (FIG. 4f). Remarkably, NC_HEE_D1 does not denature in 6 M GdnHCl (FIG. 4g, top row). Treatment with TCEP causes unfolding of all three designs, highlighting the importance of disulfide bonds.

Both the genetically-encoded and non-canonical disulfide crosslinked designs were created de novo without sequence information from natural proteins. Searches for similar sequences in the Protein Database (PDB) and National Center for Biotechnology information (NCBI) non-redundant database using PSI-BLAST found a significant alignment (e-value <0.01) only for NC_EHE_D1. This sequence has weak similarity (e-value of 2×10⁻⁴) to the zinc-finger domain of lysine-specific demethylase (PDB ID: 2MA5), but the aligned regions adopt different structures. (FIG. 11)

Synthetic Backbone-Cyclized Peptides

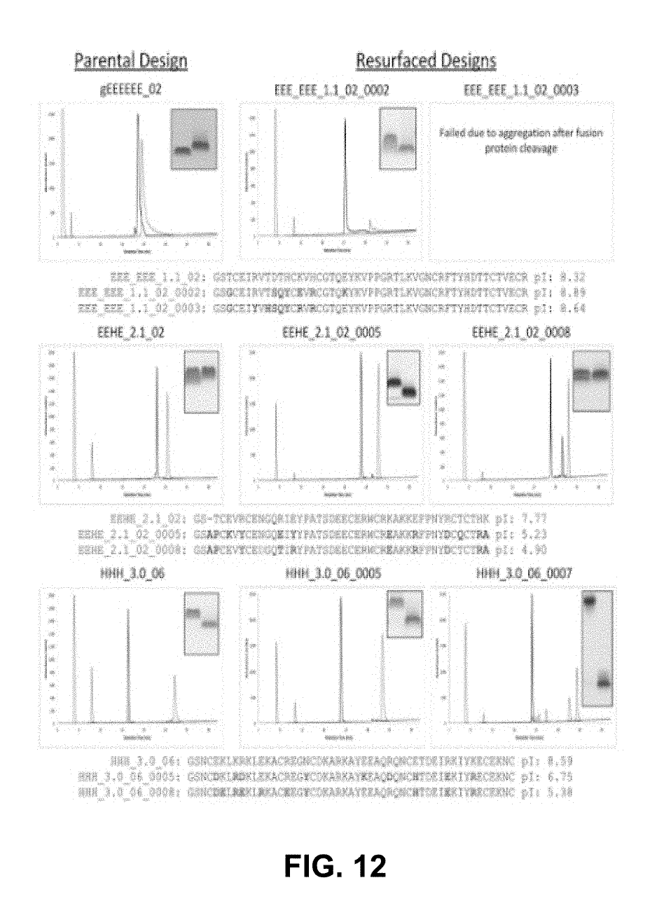

Next, the design of peptides with cyclized backbones was explored, which can increase stability and protect against exopeptidases. To generate such backbones without dependence on fragments of known structures, a GenKIC technique was implemented to sample arbitrary covalently-linked atom chains capable of connecting the termini. Each GenKIC chain-closure attempt involves perturbing multiple chain degrees of freedom, then analytically solving kinematic equations to enforce loop closure with ideal peptide bond geometry in the case of N—C cyclic peptides (see Experimental Methods, FIG. 12). Sequence design, backbone relaxation, and in silico structure validation using MD simulation and Rosetta™ ab initio structure prediction were carried out with terminal bond geometry constraints (FIG. 9).

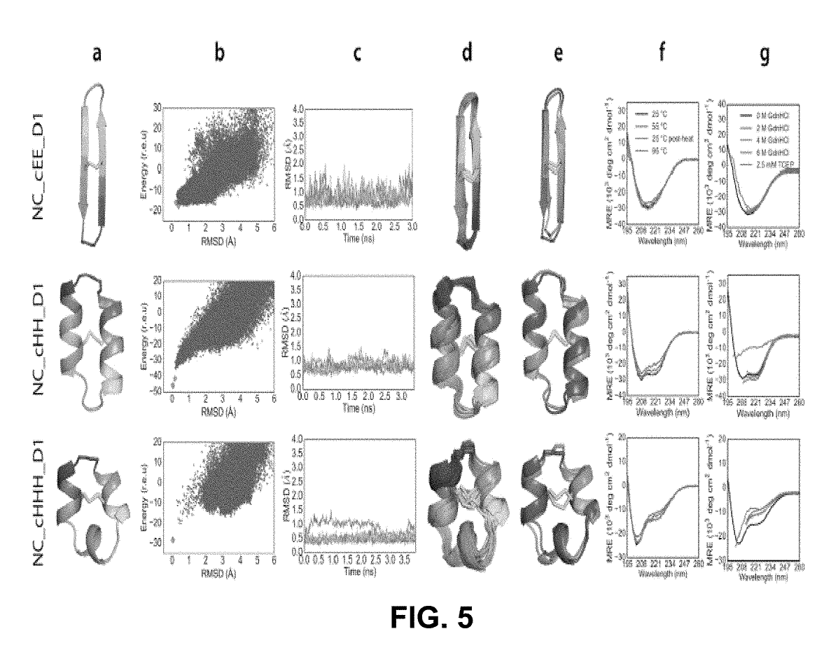

Cyclic peptides for three topologies (cEE, cHH, and cHHH) were synthesized and their structures were determined by NMR spectroscopy. The 18-residue NC_cEE_D1 design has a cyclic anti-parallel β-sheet fold similar to natural theta-defensins, but with one (rather than three) disulfide bonds, and non-canonical turns. The lowest-energy NMR model has a C_{α} RMSD of 1.26 Å to the designed structure. The variability in the curvature of the sheets across the NMR ensemble is similar to the variability observed in the structure calculations (FIG. 5, top row). The 26-residue NC_cHH_D1 design, which has one disulfide bond linking the two α-helices, has a 1.03 Å C_{α} RMSD from the lowest-energy NMR structure (FIG. 5, second row). The 22-residue NC_cHHH_D1 design has three short regions of α-helical structure and a single disulfide bond. The NMR structure of the design was again very close to the design model (FIG. 5, third row), with a C_{α} RMSD of 1.06 Å to the lowest-energy NMR structure.

All three cyclic topologies were found to be extremely stable in thermal denaturation experiments, retaining CD signal when heated to 95° C. (FIG. 5f). The CD spectra of NC_cHH_D1 and NC_cEE_D1 were nearly identical in 0 and 6 M GdnHCl, indicating that these peptides do not chemically denature (FIG. 5g; NC_cHHH_D1 showed some loss of secondary structure in 6M GdnHCl). After treatment with TCEP, both NC_cHH_D1 and NC_cHHH_D1 lost secondary structure, but the CD spectrum of NC_cEE_D1 was not changed by reduction of the central disulfide bond (FIG. 5g, top row). Overall, the cyclic designs are exceptionally stable given their very small sizes.

Beyond Natural Secondary and Tertiary Structure

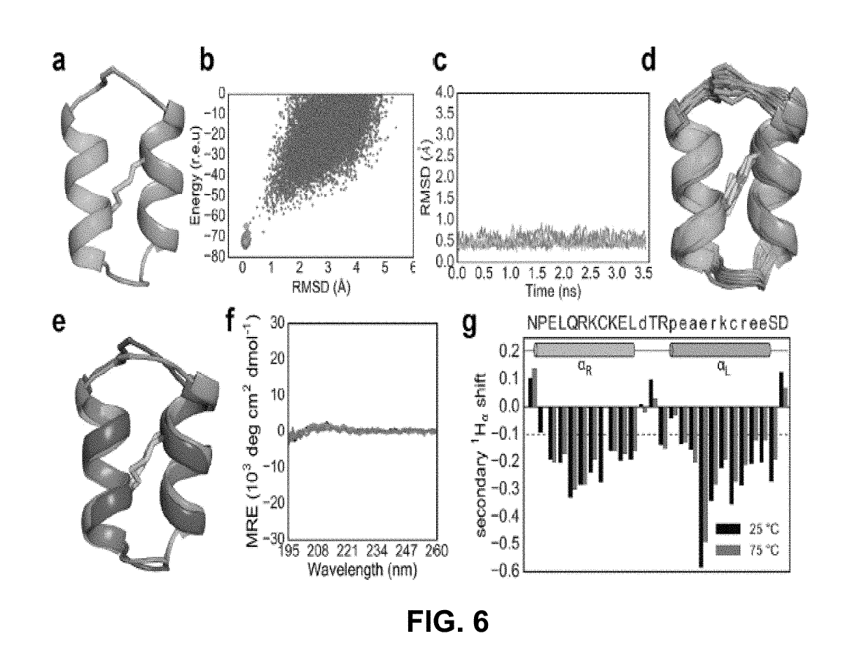

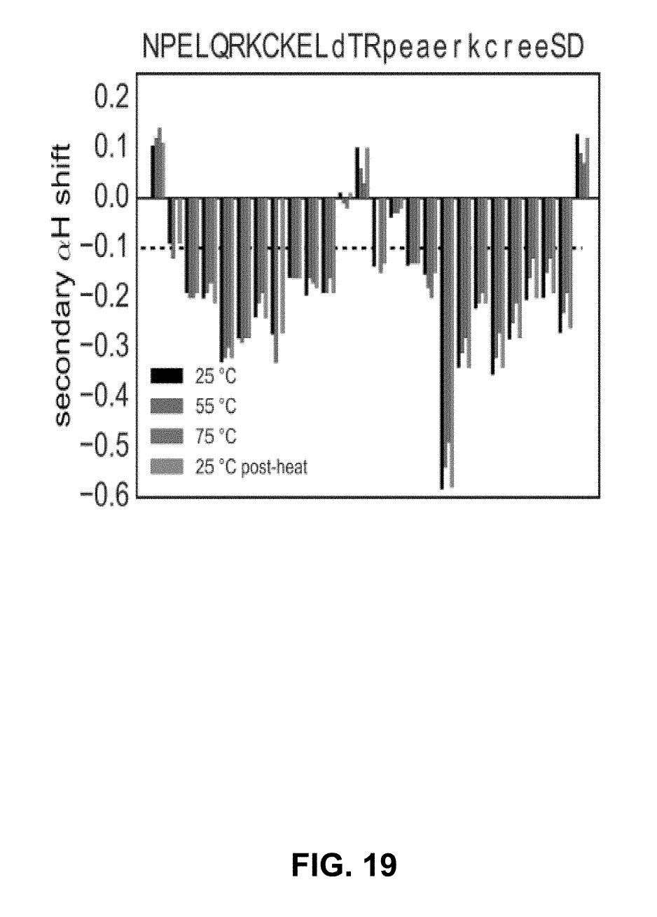

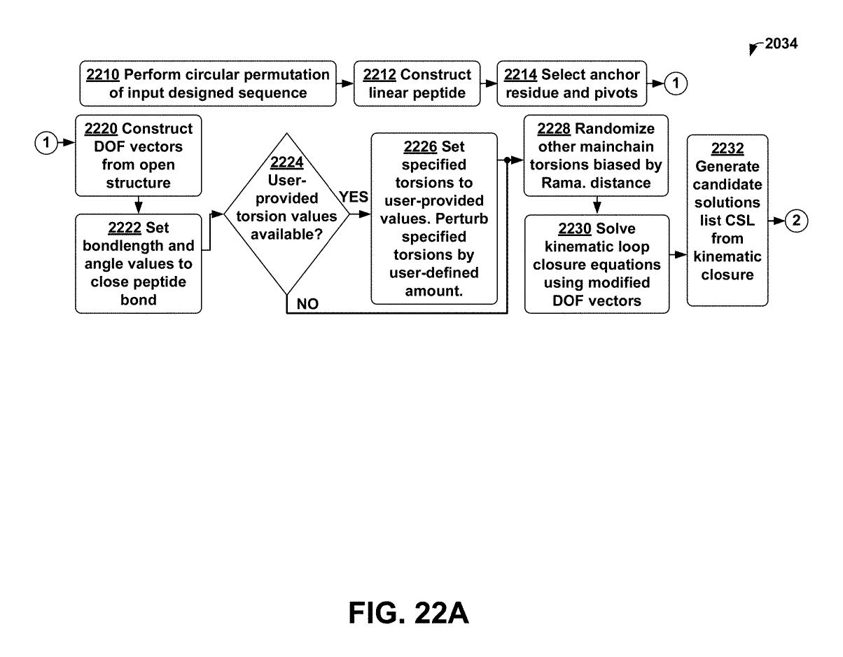

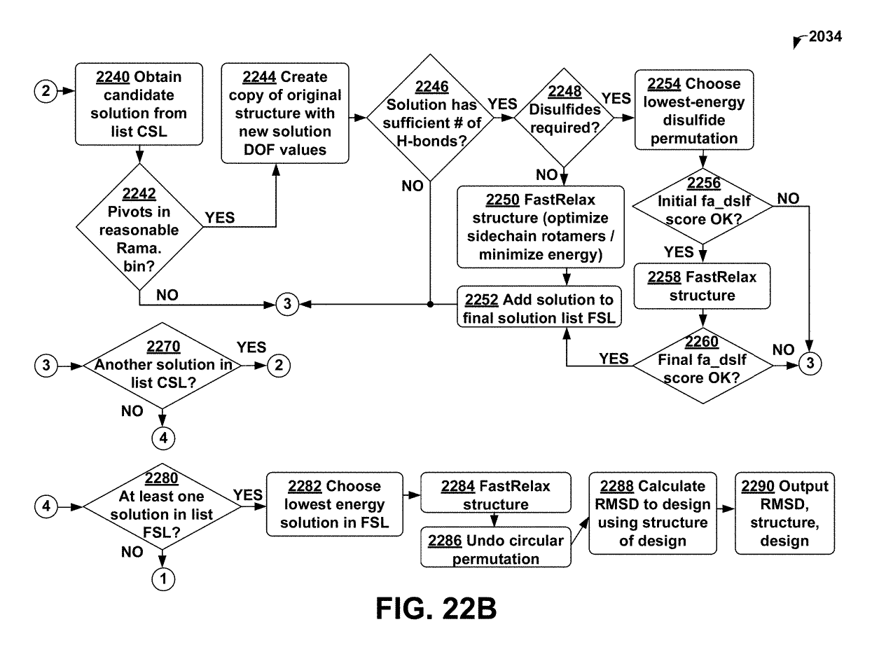

As a final test of the generality of the new design methodology, a heterochiral, backbone-cyclized, two-helix topology with one non-canonical left-handed α-helix and one canonical right-handed α-helix (H_{L}H_{R}) assembling into a tertiary structure not observed in natural proteins was designed. As before, designs were validated by MD; however, for validation by ab initio structure prediction it was necessary to develop a new, GenKIC-based structure prediction protocol (see Computational Methods, and FIGS. 22A, 22B) since the standard Rosetta™ ab initio structure prediction method utilizes fragments of native proteins, which typically do not contain left-handed helices. A selected design for this topology, NC_H_{L}H_{R}_{_}D1, is a 26-residue peptide with one D-cysteine, L-cysteine disulfide bond connecting the right-handed and left-handed α-helices. There is an excellent match between the NMR structure ensemble and design model (C_{α} RMSD: 0.79 Å) (FIG. 6). As expected for the nearly achiral topology, the CD signal is very small (as observed for a previously-studied two-chain, four-helix mixed D/L system), and no change was observable on heating to 95° C. The secondary ¹H_{α} chemical shifts also show no significant change on heating to 75° C. (FIGS. 6g and 19), indicating that the peptide is thermostable. Successful design of this topology demonstrates that these computational methods are sufficiently versatile and robust to design in a conformational space not explored by nature.

The key advances in computational design presented here—notably the methods for designing constrained peptide backbones spanning a broad range of topologies and incorporating natural and non-natural building-blocks—enable high-accuracy design of new peptides with exceptional thermostability and resistance to chemical denaturation. All twelve experimentally-determined structures are in close agreement with the design models, including one with helices of different chirality. Unlike the natural constrained peptide families, designed peptides are not limited to particular shapes, sizes, nucleating motifs, or disulfide connectivities; indeed, the sequences of these de novo peptides are quite different from those of any known peptides. In some examples, the herein-described techniques can be used for extending sampling and scoring methods to permit design with D-amino acids and cyclic backbones. In other examples, the herein-described techniques can fully generalized to peptides containing more exotic building-blocks, such as amino acids with non-canonical sidechains or non-canonical backbones.

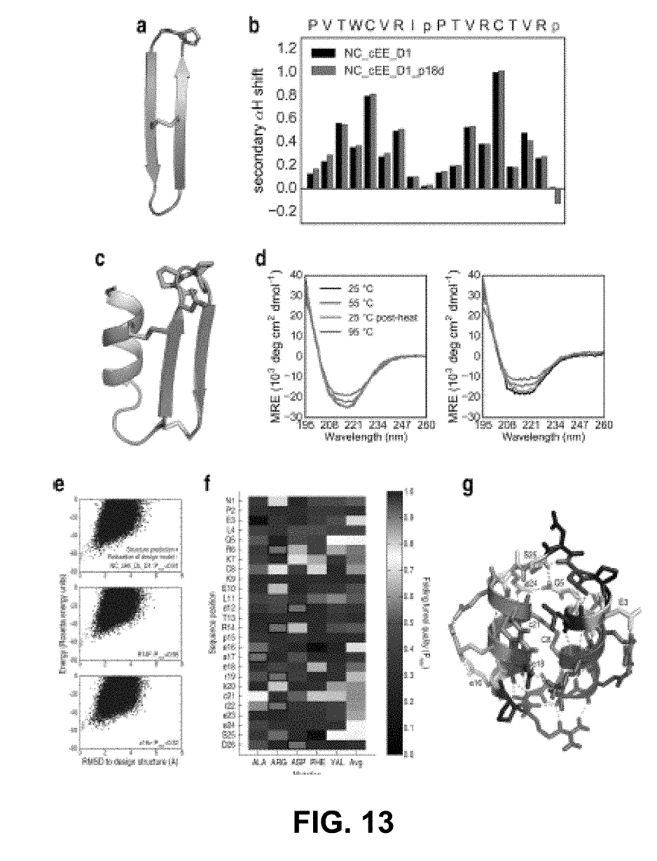

The hyperstable molecules presented in this study provide robust starting scaffolds for generating peptides that bind targets of interest using computational interface design or experimental selection methods. Solvent-exposed hydrophobic residues can be introduced without impairing folding or solubility (FIGS. 12, 13, 19) suggesting high mutational tolerance. Hence it should be possible to reengineer the peptide surfaces, incorporating target-binding residues to construct binders, agonists, or inhibitors. There has been considerable effort in both academia and industry to employ small, naturally-occurring proteins as alternatives to antibody scaffolds for library selection-based affinity reagent generation. These genetically-encoded designs offer considerable advantages as starting points for such approaches because of their high stability, small size, and diverse shapes. Furthermore, having been designed exclusively to be robust and stable, they lack the often-destabilizing non-ideal structural features that arise in naturally occurring proteins from evolutionary selective pressure for a particular function. Similarly, the heterochiral designs described here provide starting points for split-pool and other selection strategies compatible with non-canonical amino acids.

Going beyond the reengineering of hyperstable designs to bind targets of interest, the methods developed herein can be used to design new backbones to fit specifically into target binding pockets. Such “on-demand” target-specific scaffold generation is likely to yield scaffolds with considerably greater shape-complementarity than that of scaffolds generated without knowledge of the target. More generally, these computational methods open up previously inaccessible regions of shape space, and, in combination with computational interface design, should help unlock the pharmacological potential of peptide-based therapeutics.

II. Experimental Methods

Protein Purification of Genetically-Encodable Disulfide-Rich Peptides

Genes of designed disulfide-rich peptides were cloned into the vector pCDB180 (available via Addgene) using Gibson Assembly. Protein expression from E. coli was carried out using a large N-terminal fusion domain consisting of: the native E. coli protein OsmY to direct periplasmic and extracellular localization, a deca-histidine tag for protein purification, and the SUMO protein Smt3 from Saccharomyces cerevisiae to chaperone folding and provide a mechanism for scarless cleavage of the fusion from the designed protein. Designed proteins were expressed from BL21*(DE3) E. coli (Invitrogen), and expression cultures were grown overnight with incubation at 37° C. and shaking at 225 RPM. Following expression via Studier autoinduction, a periplasmic extract was prepared by washing cells with: 20% sucrose, 30 mM Tris-HCl pH 8.0, 1 mM EDTA pH 8.0, 1 mg/mL lysozyme. Protein was purified from the bacterial-conditioned medium and/or the periplasmic extract by immobilized metal-affinity chromatography (IMAC). During screening, fusion protein was purified from the bacterial-conditioned medium of 50 mL cultures, which typically yielded 9±4 mg of protein (prior to removal of the fusion protein). Protein expression from mammalian cells was carried out using the Daedalus system, as previously described in detail. With both purification systems, purified fusion proteins were cleaved by a site-specific proteins, SUMO protease for E. coli and TEV protease for Daedalus, followed by a secondary IMAC step. The final designs were purified to homogeneity by reverse-phase high-performance liquid chromatography on an Agilent 1260 HPLC equipped with a C-18 Zorbax SB-C18 4.6×150 mm column. Solvent A (Water+0.1% TFA) and solvent B (Acetonitrile+0.1% TFA) were run using the following gradient: 0-5% solvent B (5 minutes), 5-45% solvent B (40 minutes).

Synthesis and Purification of Non-Canonical Peptides

Linear and cyclic peptides were synthesized as previously described. Briefly, peptides were synthesized using automated solid phase peptide synthesis with Fmoc (9-fluorenylmethyloxycarbonyl) strategy. Cyclic reduced peptides were obtained after cleavage of the sidechain-protected peptides from the resin, ligation of both termini and the cleavage of sidechain protecting groups. Linear reduced peptides were collected by cleaving the sidechain protecting groups and resin from the peptides simultaneously. All linear or cyclic reduced peptides were oxidized at room temperature in a buffer containing 0.1 M NH₄HCO₃, where the peptide concentration was 0.25 mg/mL. After 48 h, the mixture was acidified with trifluoroacetic acid, loaded onto a semi-preparative column and purified by RP-HPLC.

Mass Spectrometry

Intact samples for each genetically-encodable peptide were diluted in loading buffer with 0.1% formic acid and analyzed on a Thermo Scientific Orbitrap Fusion Tribrid Mass Spectrometer via data-dependent acquisition. Liquid chromatography consisted of a 60 minute gradient across a 15 cm column (75 μm internal diameter) packed with C₁₈ resin with a 3 cm kasil frit trap (150 μm internal diameter) packed with C₁₂ resin. For disulfide connectivity analysis, peptides were digested with sequencing grade modified trypsin (Promega) at 1:50, enzyme to substrate, concentration for 1 hour at 37° C. then desalted via mixed-mode cationic exchange (MCX). Peptide samples were dried under vacuum and resuspended in 0.1% formic acid. Digested samples were analyzed using both data-dependent acquisition and targeted methods.

Thermal and Chemical Denaturation Experiments

Circular dichroism (CD) wavelength and temperature scans were recorded on AVIV model 420 or Jasco J-1500 CD spectrometer. For thermal denaturation, peptides samples were prepared at 0.07-0.2 mg/ml final concentration in 10 mM sodium phosphate buffer (pH 7.0). Wavelength scans from 195 nm to 260 nm were recorded at 25° C., 55° C., 95° C., and again after cooling back to 25° C. For chemical denaturation experiments, samples for each peptide were prepared in the presence of 0 M to 6 M GdnHCl concentrations. The concentration of GdnHCl was measured by refractometry. Peptide samples were also prepared in the presence of 2.5 mM TCEP (TCEP was pre-equilibrated to pH 7.0 prior to addition), and incubated for 3 hours. Peptide concentrations were the same across all samples. Wavelength scans from 190 nm to 260 nm were recorded for each sample in 0.1 cm cuvette.

NMR Analysis and Structure Determination of Genetically-Encodable Disulfide-Rich Peptides

Agilent NMR spectrometers operating at ¹H resonance frequencies between 500 to 750 MHz equipped with ¹H{¹⁵N, ¹³C} probes were used to acquire NMR data for gEHE_06, gEEHE_02, gEEH_04, and gHHH_06. The peptides were all uniformly ¹⁵N-labeled with gEEH_04 and gHHH_06 also ˜10% labeled with ¹³C. The peptides were suspended in 50 mM sodium chloride, 20 mM sodium acetate, pH 4.8 (gEHE_06 and gEEHE_02) or 50 mM sodium phosphate, 4 μM 4,4-dimethyl-4-silapentane-1-sulfonic acid, 0.02% sodium azide, pH 6.0 (gEEH_04 and gHHH_06) at concentrations between 1.5 and 0.5 mM. The ¹H, ¹³C, and ¹⁵N chemical shifts of the backbone and sidechain resonances were assigned by analysis of two-dimensional [¹⁵N,¹H] HSQC, [¹³C,¹H] HSQC (aliphatic and aromatic), [¹H,¹H] TOCSY, and [¹H,¹H] NOESY spectra, and three-dimensional (3D)¹⁵N-resolved [¹H,¹H] TOCSY, ¹⁵N-resolved [¹H,¹H] NOESY, HNCA, HNCO, and HNHA spectra acquired at 20° C. (for gEHE_06 and gEEHE_02) and 25° C. (gEEH_04 and gHHH_06), respectively. Mixing times of 90 ms (gEHE_06 and gEEHE_02) and 200 ms (gEEH_04 and gHHH_06) were used for 2D and 3D NOESY, respectively. Slowly exchanging amides were identified for gEHE_06 and gEEHE_02 by lyophilizing a ¹⁵N-labeled protein, re-dissolving in D₂O, and collecting a 2D [¹⁵N,¹H] HSQC spectrum ˜10 minutes after re-dissolving the protein. The resulting D₂O sample was subsequently used to collect additional 2D [¹H-¹H] TOCSY and [¹H-¹H] NOESY data. Stereospecific assignments for the Val and Leu methyl groups were obtained for gEEH_04 for the 10% fractionally ¹³C-labelled sample. Because it was not economical to prepare uniformly ¹³C-labelled peptides by autoinduction, established triple-resonance NMR backbone assignment protocols could not be used. Instead, the carbon resonances were assigned by analyzing the 2D [¹H,¹H] TOCSY spectra along with [¹³C,¹H] HSQC spectra (collected at natural ¹³C abundance for gHHH_06, gEHE_06 and gEEHE_02). For gEEH_04, which was 10% fractional ¹³C-labeled, the assignments were complemented with HNCA spectra. NMR data were processed using the Felix2007 (MSI, San Diego, Calif.) and PROSA (v6.4) programs and were analyzed using the programs Sparky (v3.115), XEASY, or CARA. Proton chemical shifts were referenced to internal DSS, while ¹³C and ¹⁵N chemical shifts were referenced indirectly via gyromagnetic ratios. Chemical shifts, NOESY peak lists and time domain NMR data were deposited in the BioMagResBank (for accession numbers see Table 1).

Isotropic overall rotational correlation times of 1.6-1.3 ns were inferred from averaged backbone ¹⁵N spin relaxation times (www.nmr2.buffalo.edu/nesg.wiki), indicating that all peptides are monomeric in solution. The ¹H, ¹³C, and ¹⁵N chemical shift assignments and NOESY peak lists were used for iterative structure calculations using the program CYANA (v 2.1 and 3.97). Chemical shifts were used to derive dihedral phi and psi angle constraints using the program TALOS+ for residues located in well-defined regular secondary structure elements. For the final structure calculation, hydrogen bond restraints were also introduced for gEHE_06 and gEEHE_02, for slowly exchanging amide protons. The resulting ensemble of 20 CYANA conformers was refined by restrained molecular dynamics in an ‘explicit water bath’ using the program CNS (v1.3). Structural quality was assessed using the online Protein Structure Validation Suite (PSVS, v1.5). The structural statistics are summarized in Table 1. The coordinates for the 20 conformers representing the solution structures were deposited in the PDB (for accession numbers see Table 1).

NMR Analysis and Structure Determination of Non-Canonical Peptides

Each non-canonical peptide (1 mg) was dissolved in 500 mL of 10% D₂O/90% H₂O or 100% D₂O (˜pH 4). NMR spectra were recorded at 298K on a Bruker Avance-600 spectrometer. Two-dimensional NMR experiments included TOCSY with an 80 s MLEV-17 spin lock, NOESY (200 ms mixing time), ECOSY, as well as natural-abundance ¹³C and ¹⁵N HSQC. Solvent suppression was achieved using excitation sculpting. Spectra were processed using Topspin 2.1 then analyzed using CcpNmr Analysis. Chemical shifts were referenced to internal 2,2-dimethyl-2-silapentane-5-sulfonate (DSS).

Initial structures were generated using CYANA and were based upon distance restraints derived from NOESY spectra recorded in both 10% and 100% D₂O. The following restraints were also included: disulfide bonds, hydrogen bonds as indicated by slow D₂O exchange and sensitivity of amide proton chemical shift to temperature, chi1 restraints from ECOSY and NOESY data, and backbone phi and psi dihedral angles generated using the program TALOS-N. The final set of structures was generated within CNS using torsion angle dynamics, refinement and energy minimization in explicit solvent and protocols as developed for the RECOORD database. Final structures were assessed for stereochemical quality using MolProbity.

X-Ray Crystallography

The gEHEE_06 peptide was purified by size exclusion chromatography on an AKTA Pure using a GE HiLoad 16/600 Superdex 75 pg column, concentrated to 50 mg/ml, and crystallized by vapor diffusion over well solutions of 100 mM citrate (pH 3.5), and 25% PEG3350. Selected crystals were transferred to a cryo-solution of 100 mM citrate (pH 3.5), 20% PEG3350, and 15% glycerol. Diffraction data were collected on a Rigaku Micromax-007HF with a Saturn944+ CCD detector, and integrated and scaled with HKL-2000. Initial phases were determined by molecular replacement using Phaser as implemented in the CCP4 software suite with coordinates derived from a Rosetta™ model for the scaffold. Molecular replacement found 2 molecules per asymmetric unit (ASU). This solution was iteratively refined with the program Refmac followed by model building with COOT, yielding a crystallographic R-values (Rcryst=39.9%, Rfree=42.5%). Based on the Matthews' coefficient, the crystals should have contained 3 molecules per ASU in order to have a reasonable solvent content of 45%. At this point positive electron density appeared that allowed for the manual positioning of a third molecule in the ASU and improving the R-values (R^{cryst}=32.0%, R_{free}=34.9%). The model was further improved by including solvent molecules and TLS refinement. The quality of the final model was assessed using ProCheck and Molprobity (overall score: 100th percentile). The final model has been deposited in the PDB with accession code 5JG9. Crystallographic statistics are reported in Table 2.

Surface Redesign

In attempt to reduce solubility and enhance crystallization, solvent-exposed residues of designs representing each major topological category (mixed α/β, all β-sheet, all α-helical) were redesigned. Two resurfaced variants were selected for each design bearing between one to two solvent-exposed tyrosine residues. These resurfaced designs were then expressed and purified using Daedalus, all of which expressed solubly and exhibited a redox-sensitive migration time by reverse-phase HPLC. It was only possible to obtain diffracting protein crystals for redesign gEEHE_2.1_02_0008, which diffracted to 2.90 Å resolution (Table 2). However, Matthews calculations predicted non-crystallographic symmetry with approximately nineteen copies in the asymmetric unit, and attempts to phase the crystal by molecular replacement were unsuccessful, as were attempts at reproducing the crystal outside of the initial screen.

TABLE 1
Summary of the structural statistics for gHHH_06, gEHH_4, gEHE_06, and gEEHE_02.

| | Design | | | | | | | |
|---|---|---|---|---|---|---|---|---|
| | gHHH_06 | | gEEH_04 | | gEHE_06 | | gEEHE_02 | |
| Completeness of ¹H resonance | | | | | | | | |
| assignments^{b} (%) | | | | | | | | |
| Backbone/Side-chain | 100/90 | | 99/70 | | 96/72 | | 97/84 | |
| Conformationally-restricting | | | | | | | | |
| constraints^{c} | | | | | | | | |
| Distance Constraints | | | | | | | | |
| Total | 742 | | 614 | | 317 | | 301 | |
| intra-residue (i = j) | 224 | | 135 | | 116 | | 100 | |
| sequential (\|i-j\| = 1) | 220 | | 166 | | 102 | | 96 | |
| medium range (1 < \|i-j\| < 5) | 242 | | 156 | | 43 | | 35 | |
| long range (\|i-j\| ≥ 5) | 56 | | 157 | | 56 | | 70 | |
| Dihedral angle constraints | 54 | | 44 | | 54 | | 46 | |
| Disulfide bond constraints | 6 | | 6 | | 6 | | 9 | |
| Hydrogen bond constraints | — | | — | | 40 | | 34 | |
| No. of constraints per residue | 19.0 | | 17.8 | | 11.9 | | 10.5 | |
| No. of long range constraints | 1.5 | | 4.7 | | 1.6 | | 1.9 | |
| per residue | | | | | | | | |
| Residual constraint violations^{c} | | | | | | | | |
| Average no. of distance | | | | | | | | |
| violations per structure: | | | | | | | | |
| 0.1-0.2 Å | 9.1 | | 5.3 | | 0.4 | | 0.1 | |
| 0.2-0.5 Å | 4.75 | | 2.05 | | 0 | | 0 | |
| >0.5 Å | 0.7 | | 0 | | 0 | | 0 | |
| Average no. of dihedral angle | | | | | | | | |
| violations per structure: | | | | | | | | |
| 1-10° | 6.6 | | 4.75 | | 0.1 | | 0.35 | |
| Model Quality^{c} | | | | | | | | |
| RMSD backbone atoms (Å)^{c} | 0.51 ± 0.10 | | 0.42 ± 0.11 | | 0.55 ± 0.12 | | 0.46 ± 0.09 | |
| RMSD heavy atoms (Å)^{c} | 1.16 ± 0.11 | | 1.12 ± 0.28 | | 1.43 ± 0.11 | | 1.21 ± 0.11 | |
| RMSD bond lengths (Å) | 0.018 | | 0.021 | | 0.005 | | 0.005 | |
| RMSD bond angles (°) | 1.2 | | 1.1 | | 0.7 | | 0.6 | |
| MolProbity Ramachandran | | | | | | | | |
| statistics^{c} | | | | | | | | |
| Most favored regions (%) | 96.9 | | 96.9 | | 97.8 | | 96.5 | |
| Allowed regions (%) | 3 | | 2.6 | | 2.2 | | 3.5 | |
| Disallowed regions (%) | 0.1 | | 0.4 | | 0.0 | | 0.0 | |
| Global quality scores (Raw/ | | | | | | | | |
| Z-score)^{c} | | | | | | | | |
| Verify3D | 0.34 | −1.93 | 0.22 | −3.85 | 0.35 | −1.77 | 0.42 | −0.54 |
| Prosall | 1.38 | 3.02 | 0.67 | 0.88 | 0.78 | 0.54 | 1.14 | 2.03 |
| Procheck (phi-psi)^{c} | 0.40 | 1.89 | −0.01 | 0.28 | −0.02 | 0.24 | −0.12 | −0.16 |
| Procheck (all)^{c} | 0.16 | 0.95 | −0.09 | −0.53 | −0.04 | −0.24 | −0.19 | −1.12 |
| MolProbity clash score | 15.6 | −1.15 | 16.8 | −1.37 | 17.34 | −1.45 | 18.5 | −1.66 |
| RPF Scores^{d} | | | | | | | | |
| Recall/Precision | 0.95 | 0.92 | 0.92 | 0.87 | 0.88 | 0.91 | 0.98 | 0.93 |
| F-measure/DP-score | 0.93 | 0.75 | 0.89 | 0.72 | 0.89 | 0.55 | 0.96 | 0.82 |

| | | | | |
|---|---|---|---|---|
| BMRB accession number | 26045 | 26046 | 30067 | 30069 |
| PDB ID | 2ND2 | 2ND3 | 5JHI | 5JI4 |

^{a}Structural statistics computed for the ensemble of 20 deposited structures.
^{b}Computed using AVS software from the expected number of resonances, excluding: highly exchangeable protons (N-terminal, Lys, and Arg amino groups, hydroxyls of Ser, Thr, Tyr), carboxyls of Asp and Glu, and non-protonated aromatic carbons.
^{c}Calculated using PSVS 1.5. Average distance violations calculated using the sum over r⁻⁶.
^{d}RPF scores reflecting the goodness-of-fit of the final ensemble of structures (including disordered residues) to the NOESY data and resonance assignments.Table 1

TABLE 2
Table 2. Summary of crystallographic statistics.

| Design | gEHEE_06 | EEHE_2.1_02_0008 |
|---|---|---|
| Data Collection | | |
| Space group | P2₁ | P2₁2₁2₁ |
| a, b, c, (Å) | 34.9, 45.5, 49.7 | 68.0, 109.7, 122.7 |
| , , , (°) | 90.0, 105.1, 90.0 | |
| Resolution (Å) | 50.00-2.09 (2.13-2.09) | 50.00-2.90 (2.95-2.90) |
| Unique reflections | 8734 | 20164 |
| Average redundancy | 3.5 (2.8) | 3.3 (3.4) |
| Completeness (%) | 96.7 (78.7) | 98.7 (99.7) |
| R_{merge} (%) | 11.1 (48.0) | 21.1 (56.3) |
| I/(I) | 14.4 (2.9) | 12.0 (3.9) |
| Refinement Statistics | | |
| R_{cryst} (%) | 20.0 | |
| R_{free} (%) | 24.7 | |
| Number of atoms | | |
| Protein | 1226 | |
| Water | 75 | |
| R.M.S. Deviations | | |
| Bond lengths (Å) | 0.01 | |
| Bond angles (°) | 1.62 | |
| Ramachandran | | |
| Favored (%) | 97.8 | |
| Allowed (%) | 2.2 | |
| Generously allowed (%) | 0 | |
| Disallowed (%) | 0 | |
| PDB ID | 5JG9 | |

Highest resolution shell is shown in parenthesis.

TABLE 3
Summary of the structural statistic for NC_cHHH_D1, NC_cHH_D1, NC_cEE_D1, NC_EHE_D1, NC_HEE_D1, NC_EEH_D2,
and NC_cHLHR_D1.

| | Design | | | | | | |
|---|---|---|---|---|---|---|---|
| | NC_cHHH_D1 | NC_cHH_D1 | NC_cEE_D1 | NC_EHE_D1 | NC_HEE_D1 | NC_EEH_D2 | NC_cHLHR_D1 |
| Total No. | 131 | 207 | 119 | 229 | 312 | 220 | 223 |
| Distance | | | | | | | |
| Restraints | | | | | | | |
| Intra-residue | 70 | 84 | 59 | 87 | 100 | 85 | 107 |
| Sequential | 50 | 74 | 49 | 77 | 108 | 85 | 80 |
| Medium | 7 | 32 | 4 | 36 | 42 | 24 | 31 |
| Range, i-j < 5 | | | | | | | |
| Long Range, | 4 | 17 | 7 | 29 | 62 | 26 | 5 |
| i-j ≥ 5 | | | | | | | |
| Hydrogen bond | 6 | 24 | 16 | 18 | 20 | 20 | 16 |
| constraints | | | | | | | |
| Dihedral angle | | | | | | | |
| constraints | | | | | | | |
| phi | 18 | 21 | 14 | 20 | 21 | 20 | 12 |
| psi | 17 | 22 | 14 | 18 | 21 | 20 | 9 |
| chi1 | 7 | 9 | 3 | 8 | 8 | 5 | 5 |
| Deviations | | | | | | | |
| from idealized | | | | | | | |
| geometry | | | | | | | |
| Bond lengths | 0.008 ± 0.001 | 0.008 ± 0.000 | 0.010 ± 0.000 | 0.010 ± 0.000 | 0.010 ± 0.001 | 0.009 ± 0.009 | 0.008 ± 0.000 |
| (Å) | | | | | | | |
| Bond angles | 0.925 ± 0.064 | 1.078 ± 0.057 | 1.029 ± 0.037 | 1.075 ± 0.033 | 1.075 ± 0.045 | 1.077 ± 0.049 | 1.061 ± 0.048 |
| (°) | | | | | | | |
| Impropers (°) | 1.32 ± 0.18 | 1.24 ± 0.15 | 1.20 ± 0.13 | 1.21 ± 0.13 | 1.20 ± 0.14 | 1.14 ± 0.12 | 1.23 ± 0.14 |
| NOE (Å) | 0.005 ± 0.002 | 0.010 ± 0.002 | 0.006 ± 0.003 | 0.005 ± 0.003 | 0.011 ± 0.002 | 0.005 ± 0.003 | 0.006 ± 0.001 |
| cDih (°) | 0.100 ± 0.090 | 0.058 ± 0.070 | 0.092 ± 0.075 | 0.084 ± 0.084 | 0.098 ± 0.081 | 0.091 ± 0.069 | 0.00- ± 0.000 |
| Mean Energies | | | | | | | |
| (kcal/mol) | | | | | | | |
| Overall | −796 ± 65 | −1154 ± 74 | −475 ± 12 | −958 ± 68 | −1029 ± 57 | −985 ± 54 | −1049 ± 68 |
| Bonds | 5.1 ± 0.8 | 7.2 ± 0.7 | 7.9 ± 0.7 | 10.0 ± 1.0 | 11.2 ± 1.2 | 8.4 ± 0.7 | 6.8 ± 0.7 |
| Angles: | 20.0 ± 3.2 | 31.8 ± 3.8 | 18.8 ± 1.6 | 30.9 ± 2.5 | 31.6 ± 2.8 | 28.4 ± 3.1 | 27.9 ± 2.9 |
| Improper | 9.4 ± 2.1 | 11.6 ± 2.4 | 7.8 ± 1.3 | 11.8 ± 2.1 | 12.2 ± 2.1 | 9.6 ± 1.7 | 11.0 ± 1.9 |
| van Der | −74.7 ± 5.8 | −107.4 ± 4.7 | −64.1 ± 2.4 | −120.6 ± 6.0 | −121.8 ± 5.0 | −94.9 ± 6.3 | −100.4 ± 5.0 |
| Waals | | | | | | | |
| NOE | 0.00 ± 0.00 | 0.02 ± 0.01 | 0.01 ± 0.01 | 0.01 ± 0.01 | 0.04 ± 0.01 | 0.01 ± 0.01 | 0.01 ± 0.00 |
| cDih | 0.09 ± 0.11 | 0.05 ± 0.08 | 0.05 ± 0.07 | 0.08 ± 0.11 | 0.10 ± 0.14 | 0.07 ± 0.08 | 0.00 ± 0.00 |
| Electrostatic | −858 ± 69 | −1222 ± 75 | −523 ± 10 | −1014 ± 71 | −1086 ± 59 | −1054 ± 58 | −1118 ± 70 |
| Violations | | | | | | | |
| NOE | 0 | 0 | 0 | 0 | 0 | 0 | 0 |
| violations | | | | | | | |
| exceeding | | | | | | | |
| 0.2Å | | | | | | | |
| Dihedral | 0 | 0 | 0 | 0 | 0 | 0 | 0 |
| violations not | | | | | | | |
| exceeding | | | | | | | |
| 0.2Å | | | | | | | |
| RMS deviation | | | | | | | |
| from mean | | | | | | | |
| structure, Å | | | | | | | |
| Backbone | 1.14 ± 0.34 | 0.89 ± 0.31 | 0.63 ± 0.19 | 0.93 ± 0.33 | 1.01 ± 0.32 | 0.70 ± 0.16 | 0.70 ± 0.19 |
| atoms | | | | | | | |
| All heavy | 2.13 ± 0.35 | 2.06 ± 0.39 | 1.44 ± 0.26 | 2.01 ± 0.33 | 1.96 ± 0.33 | 1.74 ± 0.30 | 1.96 ± 0.28 |
| atoms | | | | | | | |
| Stereochemical | | | | | | | |
| quality | | | | | | | |
| Residues in | 99.2 ± 1.8 | 99.8 ± 0.9 | 92.5 ± 2.5 | 92.6 ± 2.4 | 95.4 ± 1.2 | 95.4 ± 1.2 | 83.8 ± 4.4 |
| most favored | | | | | | | |
| Rama. region, | | | | | | | |
| % | | | | | | | |
| Rama. | 0.0 ± 0.0 | 0.0 ± 0.0 | 6.2 ± 0.0 | 5.7 ± 2.0 | 4.2 ± 0.0 | 4.2 ± 0.0 | 6.9 ± 2.4 |
| outliers % | | | | | | | |
| Unfavorable | 0.7 ± 2.3 | 0.4 ± 1.2 | 0.0 ± 0.0 | 0.0 ± 0.0 | 0.2 ± 0.8 | 0.0 ± 0.0 | 0.0 ± 0.0 |
| sidechain | | | | | | | |
| rotamers, % | | | | | | | |
| Clashscore, | 7.3 ± 4.0 | 4.8 ± 2.7 | 3.7 ± 2.1 | 6.7 ± 3.2 | 8.5 ± 3.2 | 7.4 ± 2.9 | 5.6 ± 2.6 |
| all atoms | | | | | | | |
| Overall | 1.4 ± 0.2 | 1.2 ± 0.2 | 1.5 ± 0.3 | 1.8 ± 0.2 | 1.8 ± 0.2 | 1.7 ± 0.2 | 1.9 ± 0.2 |
| MolProbity | | | | | | | |
| score | | | | | | | |

Table 4 below indicates sequences of computationally designed peptides.

TABLE 4

| Design | # of | Disulfide | |
|---|---|---|---|
| Name | residues | (s) | Sequence* |
| gHH_44 | 28 | C4-C26 | AEDCERIRKELEKNPNDEIKKKLEKCQA (SEQ ID |
| | | | NO: 295) |
| gHHH_06 | 43 | C2-C26, | PCEDLKERLKKLGMSEECRQRLEKMCKEGTSEDAERM |
| | | C18-C41 | ARNCES (SEQ ID NO: 213) |
| gEHE_06 | 35 | C1-C27, | CKQRRRYRGSEEECRKYAEELSRRTGCEVEVECET |
| | | C14-C33 | (SEQ ID NO: 302) |
| gEEH_04 | 38 | C2-C17, | QCYTFRSECTNKEFTVCRPNPEEVEKEARRTKEEECRK |
| | | C9-C36 | (SEQ ID NO: 257) |
| gHEEE_02 | 41 | C8-C22, | SQETRKKCTEMKKKFKNCEVRCDESNHCVEVRCSDTK |
| | | C18-C33 | YTLC (SEQ ID NO: 263) |
| | | C28-C41 | |
| gEHEE_06 | 45 | C8-C38, | EERRYKRCGQDEERVRRECKERGERQNCQYQIRKEGN |
| | | C19- | CYVCEIRC (SEQ ID NO: 247) |
| | | C41, | |
| | | C28-C45 | |
| gEEHE_02 | 36 | C2-C35, | PCECDVNGETYTVSSSEECERLCRKLGVTNCRVHCG |
| | | C4-C19, | (SEQ ID NO: 265) |
| | | C23-C31 | |
| gEEEH_04 | 41 | C1-C41, | CRCHITSSCVRVEGDNGEEYRYCSSDEEDLRRFCKEM |
| | | C3-C34, | QKQC (SEQ ID NO: 237) |
| | | C9-C23 | |
| gEEEEEE_ | 47 | C2-C15, | TCEIRVTDTHCKVHCGTQEYKVPPGRTLKVGNCRFTY |
| 02 | | C11- | HDTTCTVECR (SEQ ID NO: 271) |
| | | C42, | |
| | | C33-C46 | |
| NC_cHHH_ | 22 | C5-C18 | NPEDCRQDPEANKSPEECKKLK (SEQ ID NO: 01) |
| D1 | | | |
| NC_cHH_ | 26 | C9-C22 | HDPEKRKECEKKYTDPKKREECKRKA (SEQ ID NO: 03) |
| D1 | | | |
| NC_cEE_ | 20 | C5-C14 | PVTWCVRIpPTVRCTVRp (SEQ ID NO: 05) |
| D1 | | | |
| NC_cH_{L}H_{R}_ | 26 | C8-C21 | NPELQRKCKELdTRpeaerkcreeSD (SEQ ID NO: 09) |
| D1 | | | |
| NC_EHE_ | 26 | C1-C21, | CQTWRrVSPEECRKYKEEYnCVRCTE (SEQ ID NO: 11) |
| D1 | | C12-C24 | |
| NC_HEE_ | 27 | C4-C18, | NDKCKELKKRYPNCEVRCDpRYEVHC (SEQ ID |
| D1 | | C14-C27 | NO: 13) |
| NC_EEH_ | 26 | C2-C11, | TCVECapVKVCRPDPEEARREAEERC (SEQ ID NO: 15) |
| D2 | | C5-C26 | |

*D-amino acids in the sequence are denoted by lower-case letters.

Additional Experimental Methods

Protein Purification

Protein expression from E. coli was carried out using a large N-terminal fusion domain consisting of: the native E. coli protein OsmY to direct periplasmic and extracellular localization, a decahistidine tag for protein purification, and Smt3 from Saccharomyces cerevisiae to chaperone folding and provide a mechanism for scarless cleavage of the fusion from the designed protein. Following expression, a peri plasmic extract was prepared by washing cells with: 20% sucrose, 30 mM Tris-HCl pH 8.0, 1 mM EDTA pH 8.0, 1 mg/ml lysozyme. Protein was purified from the bacterial conditioned medium and/or the periplasmic extract by immobilized metal-affinity chromatography (IMAC). Protein expression from mammalian cells was carried out using the Daedalus system, as previously described in detail. With both purification systems, purified fusion proteins were cleaved by a site-specific proteins, SUMO protease for E. coli and TEV protease for Daedalus, followed by a secondary I MAC step. The final designs were purified to homogeneity by reverse-phase high-performance liquid chromatography.

RP-HPLC

Purified proteins were run on an Agilent 1260 HPLC equipped with a C-18 Zorbax SB-C18 4.6×150 mm column. Solvent A (Water+0.1% TFA) and solvent B (Acetonitrile+0.1% TFA) were run using the following gradient: 0-5% solvent B (5 minutes), 5-45% solvent 8(40 minutes).

Nuclear Magnetic Resonance Spectroscopy

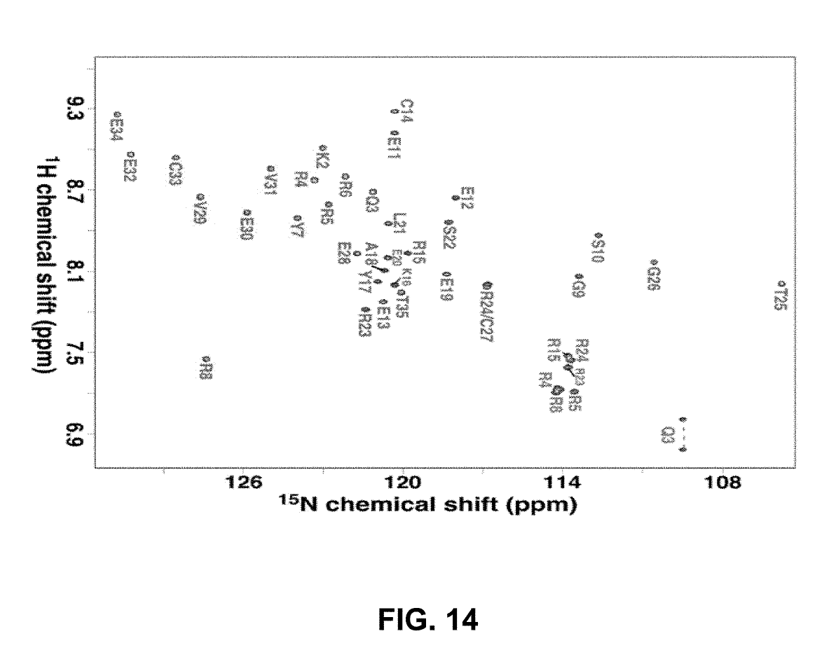

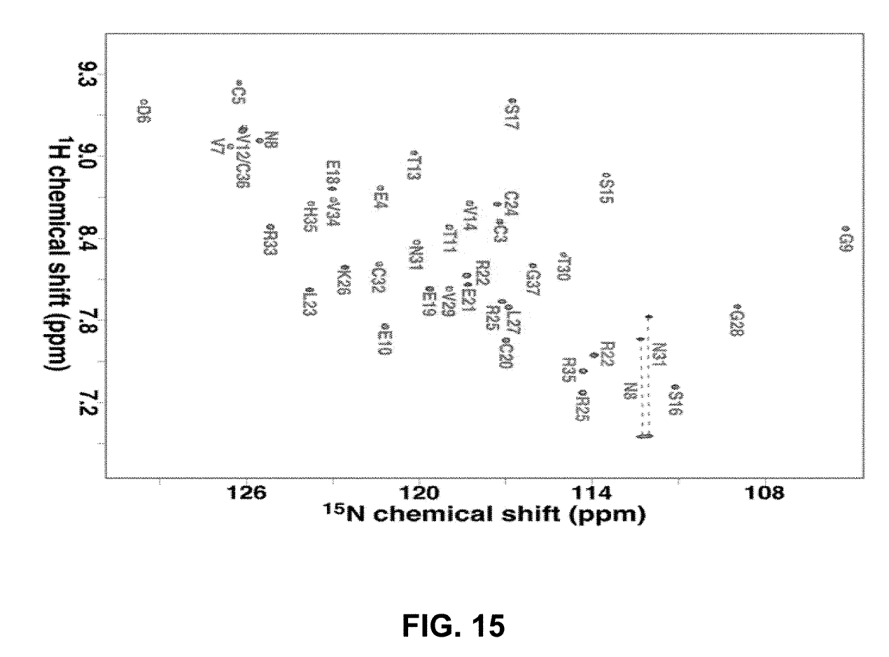

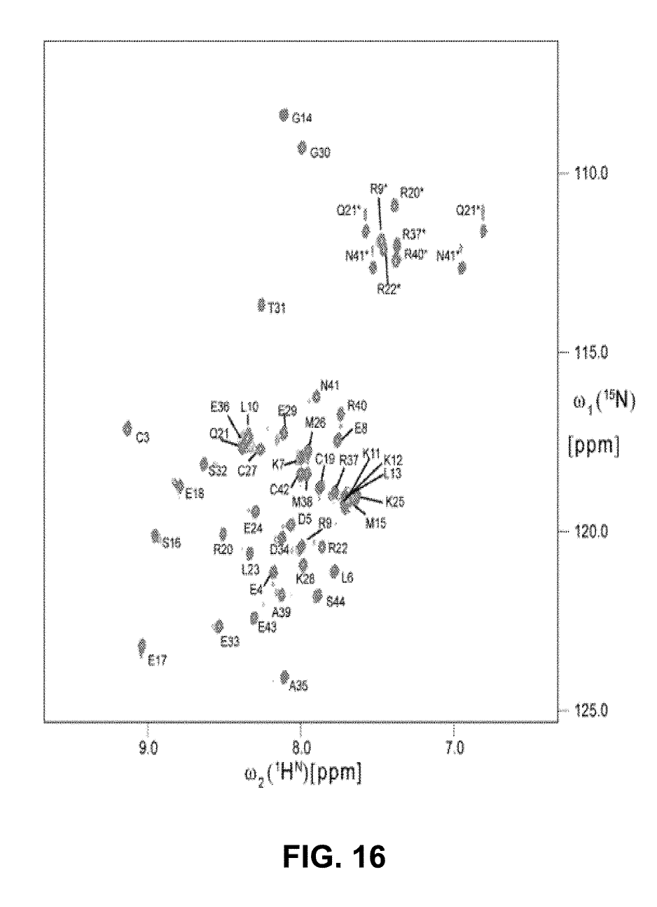

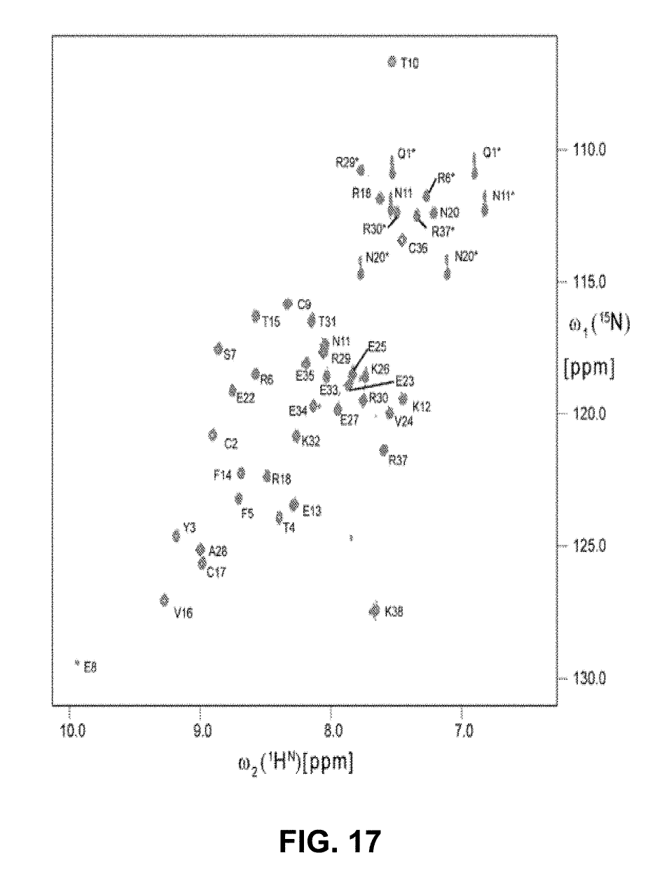

A suite of Varian NMR spectrometers with 1H resonance frequencies between 500 to 750 MHz that were equipped with HCN-probes and pulse field gradients were used to collect the NMR data for EHE_06, EEHE_02, EEH_04, and HHH_06 (FIGS. 14, 15, 16, 17). The mini-proteins were all uniformly ¹⁵N-labeled with EEH_04 and HHH_06 also ˜10% labeled with carbon-13. The miniproteins were suspended in 50 mM sodium chloride, 20 mM sodium acetate, pH 4.8 (ERE_06 and EEHE_02) or 50 mM sodium phosphate, 4 μM 4,4-dimethyl-4-silapentane-1-sulfonic acid, 0.02% sodium azide, pH 6.0 (EEH_04 and HHH_06) at concentrations that varied between 1.5 and 0.5 mM. The ¹H, ¹³C, and ¹⁵N chemical shifts of the backbone and side chain resonances were assigned from the analysis of two-dimensional ¹H-¹⁵N HSQC, ¹H-¹³C HSQC (aliph and aromatic), ¹H-¹H DPFGSE TOCSY, and ¹H-¹H DPFGSE NOESY spectra and three-dimensional ¹⁵N-edited TOCSY, ¹⁵N-edited NOESY-HSQC, HNCA, HNCO, and HNHA spectra collected at 20° C. using Varian Biopack pulse programs. A mixing time of 90 ms (EHE_06 and EEHE_02) and 200 ms (EEH_04 and HHH_06) was used to collect the NOESY data. Slowly exchanging amides were identified for ERE_06 and EEHE_02 by lyophilizing a ¹⁵N-labeled NMR sample, re-dissolving in 99.8% D₂O, and quickly collecting a ¹H-¹⁵N HSQC spectrum (˜10 minutes later). This sample in ˜100% D₂O was used to collect the H-¹H TOCSY and ¹H-¹H NOESY data. Stereospecific assignments for the Val and Leu methyl groups were made for EEH_04 and HHH_06 by observing the carbon-carbon splitting of the Pro-R methyl group in the 10% ¹³C-labelled samples (Neri et al., 1989). Because it was not economical to prepare uniformly ¹³C-labelled mini-proteins by autoinduction, traditional backbone assignment protocols could not be used. Instead, the carbon resonances were assigned by analysis of the TOCSY spectra with the ¹H-¹³C HSQC spectrum (collected with natural abundance carbon-13 for W35 and W37). For EEH_04, and HHH_06, which were 10% ¹³C-labeled, the carbon assignments were e assisted with HNCA data. All NMR data were processed using Felix2007 (MSI, San Diego, Calif.) or PROSA (v6.4) software and analyzed with the programs Sparky (v3.115), XEASY, or CARA. The ¹H, ¹³C, and ¹⁵N chemical shifts were referenced indirectly via gyromagnetic ratios (DSS=0 ppm) and deposited into the BioMagResBank (www.bmrb.wisc.edu).

NMR Structure Calculations

Isotropic overall rotational correlation times of 1.6-1.3 ns were inferred from backbone ¹⁵N spin relaxation time (www2.buffalo.edu/nesg.wiki) indicating that these miniproteins were all monomeric in solution. The 1H, ¹³C, and ¹⁵N chemical shift assignments and peak-picked NOESY data were used as initial experimental inputs in iterative structure calculations with the program CYANA (v 2.1). The assigned chemical shifts were also the primary basis for the early introduction of dihedral Psi (ψ) and Phi (φ) angle restraints (−57°±−25° (α-helix) and −139°±25° (β-strand)) and Psi (ψ) (−47°±30° (α-helix) and 140°±40° (β-strand)) identified with the CSI program (version 3.0) or TALOS+. Towards the end of the iterative structure calculation process, hydrogen (1.8-2.0 Å and 2.7-3.0 Å for the NH—O and N—O distances, respectively) disulfide (2.0-2.1 Å, 3.0-3.1 Å, and 3.0-3.1 Å for the S^{Y}-S^{Y}, S^{Y}—C^{β}, and C^{β}-S^{Y} distances, respectively) bond restraints were introduced on the basis of proximity in early structure calculations and, for the hydrogen bond restraints, the observation of slowly exchanging amides in a deuterium exchange experiment. The final ensemble of 20 CY ANA derived structures were then refined by restrained molecular dynamics in explicit water with CNS (v1.3) using the PARAM19 force field and force constants of 500, 500, and 700 kcal for the NOE, hydrogen bond, and dihedral restraints, respectively. For these water refinement calculations the upper boundaries of the CYANA distance restraints were increased up to 5% (if necessary). Structural quality was assessed using the online Protein Structure Validation Suite (PSVS, v1.5) (Bhattacharya et al., 2007). The atomic coordinates for the final ensemble of 20 structures for each mini-protein have been deposited in the Research Collaboratory for Structural Bioinformatics (RSCB).

Crystallography

EHEE_06 was purified by size exclusion chromatography on an AKTA Pure using a GE HiLoad 16/600 Superdex 75 pg column, concentrated to 50 mg/ml and crystallized by vapor diffusion over well solutions of 100 mM citrate (pH 3.5), and 25% PEG3350. Selected crystal was transferred to a cryo-solution of 100 mM citrate (pH 3.5), 20% PEG3350, with 15% glycerol, and diffraction data were collected on a Rigaku Micromax-007HF with a Saturn944+CCD detector and integrated and scaled with HKL-2000. Initial phases were determined by molecular replacement using Phaser as implemented in the CCP4 software suite with coordinates derived from a Rosetta™ model for the scaffold. Molecular replacement found 2 molecules per asymmetric unit (ASU). This solution was iteratively refined with the program Refmac followed by model building with COOT, yielding a crystallographic R-values (R^{cryst}=39.9%, R_{free}=42.5%). Based on the Matthews' coefficient, the crystals should have contained 3 molecules per ASU in order to have a reasonable solvent content of 45%. At this point positive electron density appeared that allowed for the manual positioning of a third molecule in the ASU and improving the R-values (R^{cryst}=32.0%, R_{free}=34.9%). The model was further improved by including solvent molecules and TLS refinement. The quality of the final model was assessed using ProCheck and Molprobity (overall score: 100th percentile). The final model has been deposited in the PDB with accession code 5JG9.

Surface Redesign

In attempt to reduce solubility and enhance crystallization, we performed a redesign solvent-exposed residues of designs representing each major topological category (mixed α/β, all β-sheet, all α-helical). Two re-surfaced variants were selected for each design bearing between one to two solvent-exposed tyrosine residues. We then expressed and purified these resurfaced designs using Daedalus, all of which expressed solubly and exhibited a redox-sensitive migration time by reverse-phase HPLC. We were only able to obtain diffracting protein crystals for re-design EEHE_2.1_02_0008, from topology ββαβ, which diffracted to 2.92 Å resolution. However, Matthews calculations predicted non-crystallographic symmetry with approximately nineteen copies in the asymmetric unit, and attempts to phase the crystal by molecular replacement were unsuccessful, as were attempts at reproducing the crystal outside of the initial screen.

Disulfide Positioning

To select an ideal disulfide configuration from the set of all sterically possible combinations of disulfide bonds for a given backbone, we ranked disulfide configurations according to their effect on the unfolded state configurational entropy. The reduction in unfolded state entropy due to a set of multiple cross-links was computed according to a random flight model using Eqn. 6 in Harrison et al., with ΔV=29.65 Å³ and b=3.8 Å³, as implemented in the Rosetta™ Scripts Disulfidize Mover and DisulfideEntropy Filter.

Mass Spectrometry

Multiple-Stage mass spectrometry was used to examine disulfide connectivity of the de novo miniproteins concurrent with crystallographic and NMR efforts. Purified protein samples were treated with PPS Silent Surfactant (Expedeon) and digested with Sequencing Grade

Modified Trypsin (Promega) for one hour. Sample were desalted via MCX (mixed-mode cationic exchange) and analyzed with a Thermo Scientific Orbitrap Fusion Tribrid Mass Spectrometer.

III. Computational Techniques

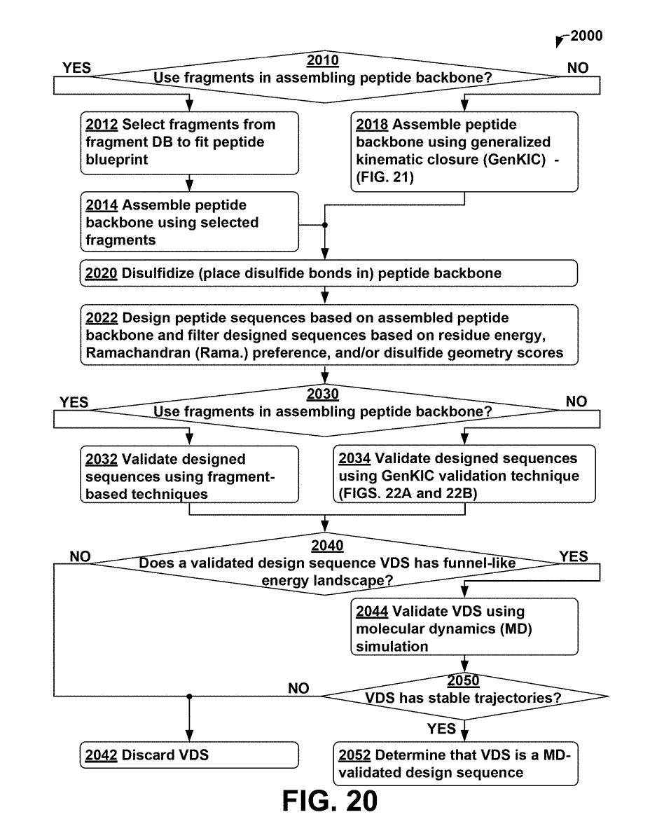

FIG. 20 shows a flowchart of a method 2000 for designing non-canonical cyclic peptides. Method 2000 can be carried out by a computing device, such as computing device 2400 described below.

De novo design of constrained peptides can be divided into two main steps: backbone assembly and sequence design. Practically, a peptide design pipeline has been optimized to permit these two steps to be performed in immediate succession with a single set of inputs, with no need for export or manual curation of generated backbones prior to the sequence design. (A third and final validation step is typically performed separately.)

For backbone assembly, two different approaches were used: disulfide-constrained topologies were sampled using a fragment assembly method, while backbone-cyclized peptide topologies were sampled using a fragment-independent kinematic closure-driven approach. Example scripts and command lines for each step in the design workflow are provided below.

Method 2000 utilizes both approaches for backbone assembly. Method 2000 can begin at block 2010. At block 2010, the computing device can determine whether to use fragments in assembling the peptide backbone (e.g., use the fragment assembly approach) or not to use fragments (e.g., use the fragment-independent kinematic closure-driven approach). For example, the computing device can determine whether to use fragments based on user input.

If the computing device determines to use fragments, the computing device can proceed to block 2012; otherwise, the computing device can proceed to block 2018.

Backbone Design Using Fragment Assembly

At block 2012, the computing device can select fragments from a fragment database (or another source) to fit a peptide blueprint. And, at block 2014, the computing device can assemble a peptide backbone using the selected fragments.

In the case of disulfide-crosslinked designs, a topology can be defined using the peptide blueprint, which specifies secondary structure and torsion bins for each amino acid residue, the latter defined using the ABEGO alphabet system described previously. The ABEGO nomenclature assigns a letter to each of five regions, or bins, in Ramachandran space. These correspond to the α-helical region (A), the β-sheet region (B), the region with positive phi values typically accessed by glycine (G), and the remainder of the Ramachandran space (E). (The fifth bin, O, represents residues with cis-peptide bonds, and was not used here.)

The blueprint is the input for a Rosetta™ Monte Carlo-based fragment assembly protocol that generates backbone conformations matching the blueprint architecture. Briefly, the fragment assembly protocol uses the defined blueprint to pick backbone fragments from a database of non-redundant high-resolution crystal structures. The insertion of fragments serves as the moves in a Monte Carlo search of backbone conformation space. For searches of the EEH topology, loop types were limited to ABEGO bins EA and GG for the ββ connection, and BAB and GBB for the αβ connection. For sampling of the EHE topology, βα connections were limited to GBB, BAB, and AB, while αβ connections were limited to GB, GBA, and AGB. For sampling of the HEE topology, αβ connections were limited to BAAB, GB, GBA, and AGB, while ββ connections were limited to EA and GG.

Upon completion of block 2014, the computing device can proceed to block 2020.

Backbone Design Using Generalized Kinematic Closure

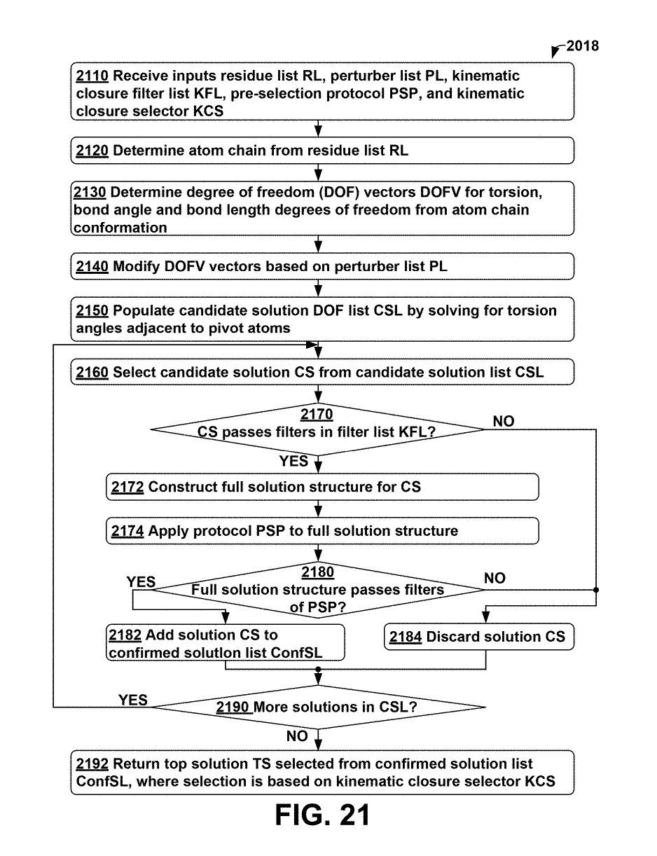

At block 2018, the computing device can assemble a peptide backbone using a GenKIC algorithm. The GenKIC algorithm is summarized immediately below and also discussed in the context of FIG. 21.

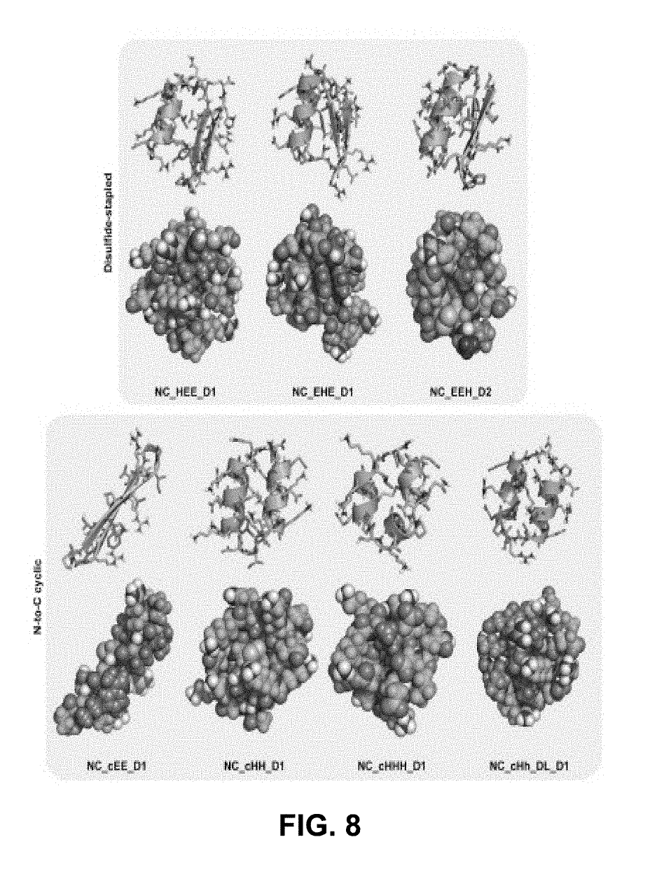

While the fragment-based approaches described above are powerful, they are limited to conformations favored by peptides composed primarily of L-amino acids. For N—C cyclic designs—NC_cHHH_D1, NC_cHH_D1, NC_cEE_D1, NC_cH_{L}H_{R}_{_}D1 (FIG. 8)—fragment-independent methods that are better suited to explore conformations that are only accessible to mixed D/L peptides were used; e.g., GenKIC-based sampling techniques.

GenKIC-based sampling works by treating a peptide as a loop, or series of loops, to be “closed”. The torsion values of an initial, “anchor” residue are randomly selected; this residue is then fixed, and the rest of the peptide is treated as a loop closure problem. The particular covalent linkages serve as a set of geometric constraints for loop closure. The GenKIC algorithm performs a series of user-controlled perturbations to the torsion angles of the peptide chain, which inevitably disrupt the geometry of the closure points. GenKIC then mathematically solves for the value of six “pivot” torsion angles that restore the geometry of the closure points and permit the loop to remain closed. Since the algorithm can return up to sixteen solutions per closure attempt, filters are applied to eliminate solutions with pivot amino acid residues in energetically unfavorable regions of Ramachandran space or with other geometric problems, such as clashes with other residues. The “best” solution is then chosen based on the Rosetta™ score function.

During the sampling steps, regions in the designed topology that were intended to form helices or sheets were initialized to ideal phi/psi values, and were either kept fixed or perturbed by only small amounts (<20 degrees). In loop regions, the perturbation was carried out by drawing torsion values randomly, biased by the Ramachandran preferences of the amino acid residue. Glycine or D/L alanine was used for backbone sampling prior to design. The allowed torsion value range either covered the entire Ramachandran space, or, in cases in which known loop ABEGO patterns could connect secondary structure elements, the mainchain torsion values were limited to those ABEGO bins. For example, during the design of the cEE topology, connection types were limited to the ‘GG’ and ‘EA’ torsion bins for the 2-residue loops.

Disulfide Positioning

At block 2020, the computing device can disulfidize (place disulfide bonds in) the peptide backbone.

To design disulfide bonds, all residue pairs with C_{β} atoms ≦5 Å apart for geometry suitable to disulfide bond formation were evaluated, backbones that could harbor disulfide bonds with near-ideal geometry were selected, and one to three disulfide bonds incorporated. To select an ideal disulfide configuration from the set of all sterically possible combinations of disulfide bonds for a given backbone, disulfide configurations were ranked according to their effect on the unfolded state configurational entropy. The reduction in unfolded state entropy due to a set of multiple crosslinks was computed according to a random flight model using Eq. 6 in Harrison et al., with ΔV=29.65 Å³ and b=3.8 Å³. This method has been implemented in the Rosetta™ software suite as the Disulfidize Mover and DisulfideEntropy Filter, both of which are accessible to the Rosetta™ Scripts scripting language.

Modifications to Rosetta™ to Permit Design of Cyclic Backbones and Mixed D/L Peptides

At block 2022, the computing device can design peptide sequences based on the assembled peptide backbone and filter the designed sequences; e.g., filter a sequence based on residue energy, Ramachandran preference, and/or disulfide geometry scores.

D-amino acid residues allow access to regions of conformational space normally only accessed by glycine. When placed correctly, they can provide greater rigidity than glycine, stabilizing glycine-dependent structural motifs and, thereby, the overall fold. Because the Rosetta™ software suite has primarily been used for designing proteins consisting of the 19 canonical L-amino acids and glycine, a number of modifications were necessary in order to permit robust design of peptides containing mixtures of D- and L-amino acids. First, Rosetta™'s default scoring function (talaris2013 at the time of the work described here) was updated to permit D-amino acids to be scored with mirror symmetry relative their L-counterparts. Terms in the score function that are based on mainchain or sidechain torsion values were modified to invert D-amino acid torsion values before applying the equivalent L-amino acid potentials. Those score function terms that are based on interatomic distances required minimal changes. To permit energy minimization, score function derivatives were also modified to invert torsion derivative values for D-amino acids. Rosetta™'s rotameric search algorithm, the packer, was modified to use L-amino acid rotamers with sidechain chi torsion values inverted for D-amino acid rotamer packing, and to update H_{α} and C_{β} positions appropriately when inverting residue chirality. Finally, an option was added to symmetrize the energy tables for the mainchain torsion preferences of glycine, which are asymmetric by default because they are based on statistics taken from the Protein Data Bank. (Glycine, in the context of L-amino acids only, occurs disproportionately in the positive-phi region of Ramachandran space, but should have no asymmetric preferences in a mixed D/L context.)

Because Rosetta™ has traditionally been used to build linear polymers, a number of core Rosetta™ libraries had to be modified to permit N—C cyclic geometry to be sampled and scored properly. The assumption that residue i is connected to residues i+1 and i−1, which is invalid for cyclic peptides, has been removed and replaced with proper lookups of connected residue indices. Cyclic geometry support was tested by confirming that the circular permutations of cyclic peptide models score identically.

Note that, as of 11 Mar. 2016, the default Rosetta™ score function has been changed to talaris2014, which re-weights a number of score terms and introduces one new term. The talaris2014 score function has also been made fully compatible with D-amino acids and cyclic geometry. A newer, experimental score function, currently called beta_nov15, has also been made fully compatible with D-amino acids and cyclic geometry.

Sequence Design and Filtering

Backbone assembly using fragment assembly or GenKIC was followed by a sequence design step. Sequence design was performed using the FastDesign protocol. This involves four rounds of alternating sidechain rotamer optimization (during which sidechain identities were permitted to change) and gradient descent-based energy minimization. The best-scoring structure was taken from a minimum of three repeats of FastDesign (twelve rounds of rotamer optimization and minimization). Each amino acid position was sorted into a layer (“core”, “boundary”, or “surface”) based on burial, and the layer dictated the possible amino acid types allowed at that position. Hydrophobic amino acid residues, for example, were only permitted at core positions. To favor more proline residues during sequence design, the reference weight for proline in the Rosetta™ score function was reduced by 0.5 units. Backbones were allowed to move during the relaxation steps. For each topology ˜80,000 structures were generated, and filtered based on the overall energy per residue, score terms related to backbone quality, and score terms related to the disulfide geometry. In a few cases for non-canonical peptides, a conservative mutation was manually introduced into a surface-exposed repeat sequence (e.g. an arginine to break a poly-lysine sequence) to facilitate unambiguous NMR assignment.

Rosetta™-Based Computational Validation

At block 2030, the computing device can determine whether to use fragments in assembling the peptide backbone or not to use fragments. For example, the computing device can determine which approach to use by using the same techniques as used at block 2010.

If the computing device determines to use fragments, the computing device can proceed to block 2032; otherwise, the computing device can proceed to block 2034.

At block 2012, the computing device can validate one or more sequences designed at block 2022 using fragment-based techniques.

Typically, the number of designs that can be created in silico exceeds the number that can be produced and examined experimentally. Rosetta™ was used to prune the list of designs, by one of two methods. For design consisting of canonical amino acids provided as fragments, Rosetta™'s fragment-based ab initio algorithm was utilized to predict a design's structure given its amino acid sequence, and to determine whether the target structure was a unique minimum in the conformational energy landscape. Disulfide bonds were not allowed to form during these simulations; the designed disulfide bonds are intended to stabilize the folded conformation rather than direct protein folding. Designs which incorporate short stretches of D-amino acids were also validated using Rosetta™'s fragment-based ab initio algorithm; the amino acid sequences of designs, with all D-amino acids mutated to glycine, were provided as input, and Rosetta™ was allowed to generate on the order of 30,000 predicted structures as output. Unlike the standard ab initio protocol, secondary structure predictions were not used in fragment picking. Additionally, the length of small and large fragments was set to 4 and 6 amino acid residues, instead of the default 3 and 9; as use of 4 and 6 amino acid residues was found to produce better sampling for peptides. After conformational sampling, the D-amino acid positions were changed to their original identities, and rescored. A small modification to the ab initio algorithm permitted it to build a terminal peptide bond for the N—C cyclic designs during the full-atom refinement stages of the structure prediction. Those designs that showed no sampling near the design conformation, or for which the design conformation was not the unique, lowest-energy conformation, were discarded.

Upon completion of block 2032, the computing device can proceed to block 2040.

At block 2034, the computing device can validate one or more sequences designed at block 2022 using a GenKIC algorithm. The GenKIC validation algorithm is summarized immediately below and also discussed in the context of FIGS. 22A and 22B.

Since fragment-based methods are poorly suited to the prediction of structures with large amounts of D-amino acid content, such as NC_cH_{L}H_{R}_{_}D1, a new, fragment-free algorithm was developed for validation of these topologies. This algorithm, called “simple_cycpep_predict”, uses the same GenKIC-based sampling approach used to build backbones for design, with additional steps of filtering solutions based on disulfide geometry, optimizing sidechain rotamers, and gradient-descent energy minimization. Because the search space is vast, even with the constraints imposed by the N—C cyclic geometry and the disulfide bond(s), the search was further biased by setting mainchain torsion values for residues in the middle of the helices to helical values (a Gaussian distribution centered on phi=−61°, psi=−41° for the α_{R} helix and on phi=+61°, psi=+41° for the α_{L} helix); this is analogous to the biased sampling obtained by fragment-based methods, in which sequences with high helix propensity are sampled primarily with helical fragments. As with ab initio validation, designs showing poor sampling near the design conformation or poor energy landscapes were discarded.

Molecular Dynamics-Based Computational Validation

At block 2040, the computing device can determine whether a validated design sequence VDS has a funnel-like energy landscape. For example, the computing device can determine a Pₙₑₐᵣ value for validated design sequence VDS, where Pₙₑₐᵣ is discussed below in the “Prediction of mutational tolerance” section. Then, if the Pₙₑₐᵣ value exceeds a threshold value (e.g., Pₙₑₐᵣ>0.5, 0.85, 0.9, or some other predetermined value), then VDS can be considered to have a funnel-like energy landscape.

If VDS has a funnel-like energy landscape, the computing device can proceed to block 2044.

Otherwise, the computing device can proceed to block 2042, where VDS is discarded. In some examples, method 2000 can end at block 2042. In other examples, the computing device can determine whether additional validated design sequences are available (e.g., multiple validated design sequences were generated at either block 2032 or 2034); and if additional validated design sequences are available, the computing device can select a validated design sequence as VDS and return to block 2040.

At block 2044, the computing device can use molecular dynamics simulation for VDS to generate one or more trajectories for VDS. At block 2050, the computing device can determine whether VDS has stable trajectories. If VDS does not have stable trajectories, the computing device can proceed to block 2042. If VDS does have stable trajectories, then the computing device can proceed to block 2052 and determine that VDS is a molecular-dynamically validated design sequence. The computing device can then output VDS as a molecular-dynamically validated design sequence, either to other modules within Rosetta™ or otherwise output VDS (e.g., write VDS to disk, generate a display based on VDS, generate an output indicating a molecular-dynamically validated design sequence has been found, etc.).

In some examples, method 2000 can end at block 2052. In other examples, the computing device can determine whether additional validated design sequences are available (e.g., multiple validated design sequences were generated at either block 2032 or 2034); and if additional validated design sequences are available, the computing device can select a validated design sequence as VDS and return to block 2040.

Further molecular dynamics-based validation of those designs for which the ab initio or simple_cycpep_predict algorithms predicted high-quality energy landscapes were performed. Similar to strategies described previously, multiple short and independent trajectories were used, starting with different initial velocities to analyze the conformational flexibility and kinetic stability of designed peptides. MD simulations were performed in explicit solvent conditions using the AMBER12 package and Amber ff12sb force field. A rectangular water box with 10 Å buffer of TIP3P water in each direction from the peptide was used for simulations. Sodium and chloride counterions were added to neutralize the system. The solvated system was minimized in two steps: solvent was first minimized for 20,000 cycles while keeping restraints on the peptide, followed by minimization of the whole system for another 20,000 cycles. At the start of simulations, the system was slowly heated from 0 K to 300 K under constant volume with positional restraints on the peptide of 10 kcal/(mol·Å) for 0.1 ns. For each selected peptide, 50 independent simulations starting with different initial velocities were performed. Each simulation started with the energy-minimized designed model, and was carried out for ˜3.5 ns. Periodic boundary conditions were used with a constant temperature of 300 K using the Langevin thermostat and a pressure of 1 atm with isotropic molecule-based scaling. A cutoff of 10 Å was used for the Lennard-Jones potential and the Particle Mesh Ewald method to calculate long-range electrostatic interactions. The SHAKE algorithm was applied to all bonds involving H atoms and an integration step of 2 fs was used for the simulations with amber12 PMEMD in the NPT ensemble. At the conclusion of the simulations, all the trajectories were analyzed using the Amber12 package and VMD. Fluctuations in RMSD were sought, and for the convergence (or the lack thereof) to the designed structure among all the trajectories. Distribution of RMSD values at the end of all trajectories was also analyzed, although the beginning two-thirds of each trajectory were discarded as a burn-in period. MD analyses for three designs of the same topology are shown in FIG. 8.

Prediction of Mutational Tolerance

Since the designed peptides presented in this study are intended to be used as starting points for designing binders to targets of therapeutic interest, the extent to which the designs can tolerate mutations (such as those that must be introduced to create a binding surface) was examined. Due to the computational expense of the mutational analysis, the NC_cH_{L}H_{R}_{_}D1 design was focused upon, mutating each position in sequence to each of alanine, arginine, aspartate, and phenylalanine and carrying out a full structure prediction simulation for each. These mutations covered each class of mutation (elimination of the sidechain, introduction of a positive or negative charge, introduction of a bulky aromatic sidechain, or introduction of a small aliphatic sidechain). Mutations preserved chirality (i.e. only D-amino acid to D-amino acid or L-amino acid to L-amino acid mutations were considered). Simulation runs were carried out on the Argonne Leadership Computing Facility's Blue Gene/Q supercomputer (“Mira”) using a version of the Rosetta™ simple_cycpep_predict application parallelized using the Message Passing Interface (MPI). A typical prediction run for a single mutation occupied 512 16-core nodes for 2.5 hours (approx. 20,000 CPU-hours per run), and produced on the order of 25,000 sampled, closed conformations with good disulfide geometry. For each mutation considered, 50 trajectories were also carried out in which the mainchain was perturbed slightly and relaxed. The resulting collection of samples (from structure prediction and relaxation) was then used to calculate a goodness-of-energy-funnel metric, termed Pₙₑₐᵣ, by the following Equation (1):

$${P}_{near} = \frac{{\sum }_{i = 1}^{N} {e}^{- {RMSD}_{i}^{2} / {λ}^{2}} {e}^{- {E}_{i} / ( {k}_{B} T )}}{{\sum }_{j = 1}^{N} {e}^{- {E}_{j} / ( {k}_{B} T )}} \qquad ( 1 )$$

The value of Pₙₑₐᵣ ranges from 0 (a poor funnel with low-energy alternative conformations or poor sampling close to the design conformation) to 1 (a funnel with a unique low-energy conformation very close to the design conformation). N is the number of samples, and Eᵢ and RMSDᵢ represent the Rosetta™ score and RMSD from the design structure of the i^{th} sample, respectively. The parameter controls how close a state must be to the design if it is to be considered native-like. This was set to 1 Å. Similarly, the parameter k_{B}T governs the extent to which the shallowness or depth of the folding funnel affects the score. This was assigned a value of 1 Rosetta™ energy unit. The Pₙₑₐᵣ metric provided a basis for comparison for the mutations considered.

Modifications to Rosetta™'s Scoring Function

Rosetta™'s scoring function consists of a number of individual score terms that are summed together to produce a final score. Each term models different aspects of the energy of a protein or peptide in a given conformation. In the past, peptides composed entirely of D-amino acids were designed in the context of an L-amino acid interaction partner by mirroring the entire system and using Rosetta™'s standard design tools to design an L-amino acid peptide in a D-amino acid binding partner context. This ensured that the energy function, optimized for L-amino acid design, would be appropriate for the region being designed. This is not an option for designing peptides of mixed chirality, however. For this reason, the manner in which many of the scoring function terms is calculated had to be modified to permit accurate scoring of peptides containing D-amino acids, or peptides with terminal (N—C) peptide bonds or other non-canonical connections.

First, it was necessary to modify the single-residue torsional potentials. In the talaris2013 scoring function, these terms are called rama (a Ramachandran potential dependent on the mainchain torsion angles phi and psi), p_aa_pp (a statistical potential that also yields a score based on the phi and psi torsion angles), omega (a potential that penalizes non-planar peptide bond geometry), and fa_dun (a potential that penalizes unfavorable sidechain conformations given the backbone). Each of these was modified so that it would score D-amino acid residues by inverting the relevant torsion values and using the score tables or analytical potentials for the corresponding L-amino acid. Derivative calculations, necessary for energy-minimization, were also modified so that D-amino acid derivatives would be calculated by inverting relevant torsion values, calculating derivatives as for the equivalent L-amino acid, and then inverting the derivatives to yield the appropriate D-amino acid derivatives.

The rama, omega, and p_aa_pp score terms required additional modification to ensure that mirror-image peptide models scored identically: the potentials for glycine, which were based on statistics from the Protein Data Bank, favored glycine in the region of Ramachandran space favored by D-amino acids. While glycine disproportionately favors such conformations in the context of L-amino acid proteins, in a mixed D/L context, one would expect its conformational preferences to by fully symmetric. Therefore, an option to Rosetta™ was added, controlled by an input flag (“-symmetric gly tables true”), which permits the user to specify that the scoring tables for rama and p_aa_pp, and that the functional form of the omega potential, be made symmetric. In the case of rama and p_aa_pp, this is done by averaging the probability table values for (phi, psi) and (-phi, -psi), re-normalizing, and converting probabilities to energies. In the case of omega, this is done by setting the potential minima, which are normally offset very slightly based on Protein Data Bank statistics, to 0° and 180°.

Of the longer-range interactions, the fa_atr (inter-residue attractive part of the van der Waals force), fa_rep (inter-residue repulsive part of the van der Waals term) and fa_sol (hydrophobic “force” used to model the hydrophobic effect in the absence of explicit solvent) also required minor modifications for cyclic peptides, since the functional form of these terms is altered slightly for residues that are adjacent in linear sequence. It was ensured that, rather than assuming that residue N is connected to residues N+1 and N−1 at its C- and N-terminal connection points, respectively, the scoring machinery would check which residues are connected and score them as adjacent residues based on covalent bonds rather than by indices.

Rosetta™'s fa_dslf score term, which holds disulfide-bonded cysteine S_{γ} residues together and penalizes deviations from ideal disulfide geometry, was updated to score D-Cys, D-Cys disulfide bonds by inverting torsion values; derivatives were similarly updated. The term then required some additional modifications to permit it to score and preserve disulfide geometry in mixed L-Cys, D-Cys disulfide bonds. This score term has energy minima for L-Cys disulfide bonds at values of −86.10° and 92.39° for the C_{β1}-S_{γ1}-S_{γ2}-C_{β2} dihedral angle, based on statistics from high-resolution crystal structures of disulfide-containing natural proteins, and the corresponding minima for D-Cys disulfide bonds were set to 86.10° and −92.39°, respectively. Since no such statistics are available for mixed L-Cys, D-Cys disulfide bonds, however, the minima were set to −90° and 90°. Similarly, the well depths for the two minima were set to identical values (the average of the depths of the two wells for L-Cys disulfide bonds).

The pro_close score term, which ensures that energy-minimization does not pull open proline ring, was updated to act on both D- and L-proline. A more general term, ring_close, has also been added which can be used on any non-canonical residue type that, like proline, contains a ring that could be pulled open by free rotation about single bonds in the absence of a potential holding it closed.

Finally, the amino acid reference energies to ensure that corresponding L- and D-amino acids have the same reference energy values were altered. (The reference energies are a zeroth-order correction factor to compensate for the fact that certain amino acid types can engage in larger numbers of favorable interactions than others, resulting in pathologies during design in which these residue types are disproportionately favored. By assigning a constant bonus or penalty to each type, this pathology is partially suppressed.)

Recently, the default Rosetta™ scoring function has been updated to talaris2014, which re-weights several terms and adds a new term, yhh_planarity, which is intended to hold the tyrosine hydroxyl proton in the plane of the tyrosine ring. It was ensured that this term also acts on D-tyrosine. A newer, experimental scoring function, currently called beta_nov15, has also entered testing, and may replace the current default scoring function at some point in the future. It has been ensured that new terms added in beta_nov15 are also compatible with D-amino acids, are properly differentiable for energy minimization, and are compatible with cyclic geometry, as described above. All scoring function changes have been tested by constructing, scoring, and minimizing mirror-image structures, confirming that the score matches for mirror-image structures, and by constructing and scoring cyclic permutations of cyclic peptides, confirming that the scoring is identical regardless the start and end points of the peptide. Unit tests have been added to ensure that, as the default Rosetta™ scoring function is replaced in the future, it continues to support D-amino acids and cyclic geometry fully.

Implementation of the GenKIC Algorithm

One of the core challenges in designing peptides with many covalent cross-links is sampling conformations permitted by the covalent geometry. Ideally, one would want an algorithm capable of only sampling conformations that yield good cross-link geometry, which would greatly reduce the search space. Kinematic closure approaches, which break the sampling problem into a series of loop closure problems and analytically solve for torsion values that permit loop closure, permit highly efficient constrained sampling. In order to apply this to peptides with arbitrary building blocks and staple chemistries, a generalized form of Rosetta™'s kinematic closure algorithm, called “GenKIC”, was implemented, in which loops can be defined as any covalently-linked chain of atoms, including chains passing through terminal peptide bonds, disulfide bonds, etc. A user interface accessible to the Rosetta™ Scripts scripting language was also developed to permit precise and versatile control over the sampling.

FIG. 21 shows a flowchart of a method for a generalized kinematic closure technique. In some examples, the method shown in FIG. 21 can be carried out by a computing device, such as computing device 2400. In particular, the method shown in FIG. 21 can be carried out as part of all of the procedures of block 2018 of method 2000.

At block 2110, a number of inputs are received by the computing device: a residue list RL, a perturber list PL, a kinematic closure list KFL, a pre-selection protocol PSP, and a kinematic closure selector KCS. In other examples, inputs are provided as needed; e.g., not all at one time as shown in FIG. 21.

At block 2120, the computing device can determine a covalently-linked chain of atoms that is the loop to be closed, as well as the start and end points of this chain is determined from residue list RL. At block 2130, the computing device can, given a chain with N degrees of freedom, determine degree of freedom vectors DOFV that meet a requirement that the rigid-body transform from the loop's start point to its end point must be maintained to maintain closure effectively reduces the degrees of freedom of the system by six.

At block 2140, the computing device can perturb N−6 degrees of freedom of vectors DOVF in user-specified ways; e.g., in accordance with perturber list PL.

At block 2150, the computing device can solve for the values of the remaining six degrees of freedom (the six torsion angles adjacent to three user-defined pivot atoms) used to preserve the rigid-body transform between the start and end points of the loop and add the resulting solutions to a candidate solution list CSL.

At blocks 2160, 2170, 2172, 2174, 2180, 2182, 2184, and 2190, solutions of the candidate solution list CSL are either confirmed and added to a confirmed solution list ConfSL or discarded. The size of CSL can be user-defined.

Since the system of equations solved at block 2150 can yield anywhere from 0 to 16 solutions from each attempt, each candidate solution CS can confirmed to be valid solution. At block 2170, the computing device can apply filters, such as filters from kinematic filter list KFL, prune CS if CS is an undesired solutions (e.g. due to clashing geometry, pivot atom torsion values lying outside of desired ranges, etc.)”. At block 2174, the computing device can apply other Rosetta™ algorithms that modify the structure (“movers”), to every GenKIC solution remaining (allowing things like sequence design, sidechain rotamer optimization, energy minimization, etc.) to determine a full structure for candidate solution CS. Then, at block 2180, the computing device can apply a set of user-selected filters provided as a protocol, such as pre-selection protocol PSP, to candidate solution CS, and if CS passes the protocol filters, candidate solution CS can be added as a confirmed solution to confirmed solution list ConfSL at block 2182, or CS can be discarded at block 2184.

At block 2192, the computing device can select a single, top solution from confirmed solution list ConfSL based on criteria specified by a user-defined GenKIC “selector”; e.g., kinematic closure selector KSL. The original structure is then updated with the new loop conformation determined as the top solution. The original structure can then serve as input into subsequent Rosetta™ modules or can be written to disk.

GenKIC perturbers have been created to permit torsion, bond angle, and bond length degrees of freedom to be set to user-defined values. These perturbers are called “set_dihedral”, “set_bondangle”, and “set_bondlength”, respectively. If a loop starts in a broken or open conformation, these perturbers can be used to define closed geometry at a particular bond, and have been wrapped in a convenient “CloseBond” statement for ease of use from the Rosetta™ Scripts user interface. Loop torsion values can also be randomized fully (“randomize_dihedral”), perturbed slightly from a starting value (“perturb_dihedral”), or, in the case of α-amino acid mainchain torsion values, both phi and psi can be drawn randomly from the Ramachandran map-biased distribution for a given amino acid type (“randomize_alpha_backbone_by_rama”). The code has been written for versatility and extensibilty, so additional GenKIC perturbers can be added as necessary.

Similarly, GenKIC filters have been defined to discard kinematic closure solutions with clashing geometry (“loop_bump_check”), with pivot torsion values in unlikely regions of

Ramachandran space (“alpha_aa_rama_check”), or with particular amino acid residues in undesired user-defined regions of Ramachandran space (“backbone_bin”). GenKIC selectors have been implemented to select the lowest-energy solution found (“lowest_energy_selector”), a random solution from the list of solutions found (“random_selector”), or a random solution biased by the energy, with lower-energy solutions weighted more heavily (“boltzmann_energy_selector”). As with GenKIC perturbers, new GenKIC filters and selectors can be implemented easily as necessary.

At the level of the Rosetta™ source code, the GenKIC algorithm is implemented as methods of the GeneralizedKIC class, which is defined in the protocols::generalized_kinematic_closure namespace. Perturbers, filters, and selectors are defined as helper classes in the sub-namespaces protocols::generalized_kinematic_closure::perturber, protocols::generalized_kinematic_closure::filter, and protocols::generalized_kinematic_closure::selector.

In some examples, additional perturbers, filters, and selectors can be added by adding methods to the appropriate helper function.

A Fragment-Free Peptide Structure Prediction Algorithm

FIGS. 22A and 22B are a flowchart of a method for peptide structure prediction using generalized kinematic closure. In some examples, the method shown in FIG. 21 can be carried out by a computing device, such as computing device 2400. In particular, the method shown in FIGS. 22A and 22B can be carried out as part of all of the procedures of block 2034 of method 2000.

Although computational validation of peptide designs containing mixtures of D- and L-amino acids is a particular challenge, those designs with small numbers of isolated D-amino acids can be validated using the classic Rosetta™ ab initio algorithm, with D-amino acid positions mutated to glycine. Classic ab initio works by choosing sets of protein fragments from known structures based on sequence alignment, then using the insertion of these fragments as moves in a simulated annealing-based search of conformational space. For a high-quality design, the ab initio algorithm reveals an energy landscape with a unique low-energy conformation corresponding to the design conformation. Poor designs either fail to sample conformations close to the design conformation, or have alternative low-energy conformations that they can access that are revealed by the sampling. Unfortunately, peptides with long stretches of D-amino acids cannot be validated in this manner, since there exist too few solved structures of known proteins in the Protein Data Bank that have long stretches of amino acid residues in the region of Ramachandran space uniquely accessed by D-amino acids, which means that suitable fragment lists cannot be generated. With the GenKIC algorithm in hand, it was possible to implement a fragment-free, GenKIC-based conformational sampling tool that could predict lowest-energy peptide structures based on amino acid sequence.

At block 2210, the computing device can randomly circularly permute the input sequence to avoid any possible artifacts that might be introduced by having the cyclization point in a particular place. At block 2212, the computing device can construct a linear peptide with the permuted sequence. All omega torsion angles are set to 180°. At block 2214, the computing device can randomly choose an amino acid residue in the sequence that is not at either of the ends to be the “anchor” residue. The anchor residue, henceforth indexed as residue M, will be the fixed point lying outside of the chain of residues that will be treated as a loop to be closed by GenKIC. This residue's mainchain phi and psi torsion angles are randomized, biased by the Ramachandran distribution for the residue type.

At blocks 2220, 2222, 2224, 2226, 2228, 2230, 2232 of FIG. 22A and blocks 2240, 2242, 2244, 2246, 2248, 2250, 2252, 2254, 2256, 2258, 2260, 2270, 2280, and 2282 of FIG. 22B, the computing device can apply the GenKIC algorithm the loop that runs from residue M+1 (immediately past the anchor residue), through the open terminal peptide bond, to residue M−1 (immediately before the anchor residue). Pivot atoms are selected: C_{α} atoms of residues M+1 and M−1 are always chosen as pivot atoms, and the third pivot is selected randomly from the C_{α} atoms in the rest of the loop. At blocks 2220-2232, the computing device can close the terminal peptide bond with ideal peptide geometry, and randomizes all mainchain torsion values within the loop biased by the Ramachandran distribution for each residue. This random sampling was found to work well for smaller peptides (up to ˜15 residues), typically allowing sampling close to the design conformation and across a broad range of alternative conformations. For longer peptides, it is necessary to bias the sampling slightly by setting mainchain torsion values near the middle of secondary structure elements to ideal values for the secondary structure type, then adding a small random perturbation to these values, such as indicated at block 2226. Loop residues and the ends of secondary structure elements are always sampled fully randomly. At blocks 2242-2246, the computing device can apply filters to eliminate solutions with pivot residues in unreasonable regions of Ramachandran space, or solutions with fewer mainchain hydrogen bonds than a user-specified number. At blocks 2254-2260, in the case of peptides containing disulfide bonds, all disulfide permutations are attempted by the computing device, and conformations incompatible with any disulfide geometry (i.e. yielding fa_dslf scores above a given threshold) are also filtered out. At blocks 2250 and 2258, the computing device can subject each GenKIC solution passing filters to multiple rounds of the Rosetta™ FastRelax algorithm which optimizes sidechain rotamers and carries out energy minimization (including optimization of disulfide geometry, if any disulfide bonds are present). Block 2270 enables the computing device to iterate through all candidate solutions.

At blocks 2280 and 2282, the computing device can choose lowest-energy sample passing filters, circularly de-permuted by the computing device at blocks 2284 and 2286, a design is calculated by the computing device at block 2288, and RMSD, structure, and/or design are output (e.g., saved to disk) by the computing device at block 2290. After many rounds of sampling, the user may then plot the calculated energy of each sample against the RMSD to the design conformation to determine whether the design conformation represents a unique low-energy state.

The peptide structure prediction algorithm shown in FIGS. 22A and B has been implemented as a Rosetta™ protocol. It is a class named protocols::cyclic_peptide_predict:SimpleCycpepPredictApplication that can be called from other code. It also exists as a stand-alone application in the Rosetta™ applications, called simple_cycpep_predict. After compiling Rosetta™, the simple_cycpep_predict application can be invoked from the command-line as shown in the following example illustrated in Table 5 (which was used to generate the plot of energy against RMSD from the design state for the NC_cH_{L}H_{R}_{_}D1 design, shown in FIG. 6).

TABLE 5

```
<path_to_Rosetta>/Rosetta/main/source/bin/simple_cycpep_predict.
default.linuxgccrelease
  -cyclic_peptide:rand_checkpoint_file rng01.state.gz -
  cyclic_peptide:checkpoint_file check01.txt -out:file:silent
  out01.silent -cyclic_peptide:sequence_file inputs/seq.txt -
  beta_nov15 -symmetric_gly_tables true -score:weights
  beta_nov15.wts -in:file:native inputs/native.pdb -
  cyclic_peptide:genkic_closure_attempts 50 -
  cyclic_peptide:genkic_min_solution_count 1 -
  cyclic_peptide:require_disulfides true -
  cyclic_peptide:disulf_ cutoff_prerelax 2000 -
  cyclic_peptide:min_genkic_hbonds 14 -
  cyclic_peptide:min_final_hbonds 14 -
  cyclic_peptide:fast_relax_rounds 5 -
  cyclic_peptide:rama_cutoff 2.0 -
  cyclic_peptide:checkpoint_job_identifier check -mute all -
  unmute
  protocols.cyclic_peptide_predict.SimpleCycpepPredictApplica
  tion -nstruct 50000 -
  cyclic_peptide:user_set_alpha_dihedrals 3 -61 -41 180 4 -61
  -41 180 5 -61 -41 180 6 -61 -41 180 7 -61 -41 180 8 -61 -41
  180 9 -61 -41 180 16 61 41 180 17 61 41 180 18 61 41 180 19
  61 41 180 20 61 41 180 21 61 41 180 22 61 41 180 23 61 41
  180 -cyclic_peptide:user_set_alpha_dihedral_perturbation
  5.0
```

A few details are worth noting: the example shown in Table5 uses symmetric glycine Ramachandran and p_aa_pp tables (-symmetric_gly_tables true). Solutions with fewer than 14 mainchain hydrogen bonds (cyclic_peptide:min_final_hbonds 14) or rama energy term scores greater than 2.0 for pivot residues (-cyclic_peptide:rama_cutoff 2.0) will be filtered out, as will solutions with pre-minimization fa_dslf scores greater than 2000 (-cyclic_peptide:disulf_cutoff_prerelax 2000).3

Sequence Design

A Rosetta™ protocol called “FastDesign” for design of amino acid sequences for a given backbone was created. Rosetta™ designs sequences using a simulated-annealing-based approach called “packing,” where random substitutions are made using the sidechain rotamers found in the Dunbrack library, in an attempt to find the sequence with lowest possible energy for each backbone. FastDesign was created as the sequence design analog to the FastRelax protocol, which is used in structure prediction. FastRelax attempts to find an optimal pose conformation with minimal energy via both small backbone movement and sidechain rotamer packing, but does not alter the existing sequence. Briefly, each repeat of FastDesign consists of four design and minimization steps. The first is done with the Lennard-Jones repulsive term down-weighted to 0.088. This allows the sidechains to clash slightly as they search for the most optimal interactions. The repulsive term is increased in the following steps, until the final step when it is at full strength (0.42). As the repulsive term is increased, the most optimal interactions will stay in place as other interactions are broken to account for the increasing repulsive term. By default, three repeats of FastDesign were performed on each backbone. The resulting structures have improved total energy and sidechain packing (as measured by the Rosetta™ packstat filter) over an equivalent number of packing/minimization steps without alteration to the repulsive term.

Example Scripts and Inputs to Design Genetically-Encodable Peptides

Table 6 below shows an example command for running the Rosetta™ Scripts XML file shown below in Table 7 is as follows:

TABLE 6

```
<path_to_Rosetta>/Rosetta/main/source/bin/rosetta_scripts.defaul
t.linuxgccrelease
  -in:file:s <arbitrary initial pdb file>
  -parser:protocol <Rosetta Scripts file>
  -out:file:s <output pdb file name>
```

For the example command line shown in Table 6, “linuxgccrelease” can be replaced with a particular user's build and compiler (e.g. “macosclangrelease” on an Apple Macintosh system using the Clang compiler.)

Table 7 below shows an example Rosetta™ Scripts XML file for designing an EHEE topology:

```
TABLE 7
<ROSETTASCRIPTS>
  <SCOREFXNS>
  #### centroid score function used for protein backbone design
####
    <SFXN_CENTROID weights=“fldsgn_cen”>
      <Reweight scoretype=“cenpack” weight=“1.0” />
      <Reweight scoretype=“hbond_sr_bb” weight=“1.0” />
      <Reweight scoretype=“hbond_lr_bb” weight=“1.0” />
      <Reweight scoretype=“atom_pair_constraint”
weight=“1.0” />
      <Reweight scoretype=“angle_constraint” weight=“1.0”
/>
      <Reweight scoretype=“dihedral_constraint”
weight=“1.0” />
    </SFXN_CENTROID>
  #### full-atom score function used for amino acid sequence
design ####
    <SFXN_FULLATOM weights=“talaris2014” />
  </SCOREFXNS>
  <RESIDUE_SELECTORS>
    <Chain name=“chain_A” chains=“A” />
  </RESIDUE_SELECTORS>
  <TASKOPERATIONS>
  #### restrict residue identity during design by the degree
with which the residue is burned ####
    <LayerDesign name=“layer_all”
layer=“core_boundary_surface_Nterm_Cterm” verbose=“True”
use_sidechain_neighbors=“True” >
      <core>
        <all append=“M” />
      </core>
      <boundary>
        <all append=“M” />
      </boundary>
      <surface>
      </surface>
    </LayerDesign>
  #### allow disulfide bonds to repack, but do not mutate ####
    <OperateOnCertainResidues name=“no_design_disulf” >
      <RestrictToRepackingRLT />
      <ResidueName3Is name3=“CYS” />
    </OperateOnCertainResidues>
  #### do not allow non-realistic chi angles of aromatic amino
acid sidechains ####
    <LimitAromaChi2 name=“limitchi2” include_trp=“True” />
  #### restrict amino acid identity of loop regions based on
abego profile ####
    <ConsensusLoopDesign
name=“disallow_nonnative_loop_sequences” />
  #### increase the diversity of rotamers available to the
packer ####
    <ExtraRotamersGeneric name=“extra_rots” ex1=“True”
ex2=“True” />
    <OperateOnCertainResidues name=“no_repack_non-disulf” >
      <PreventRepackingRLT/>
      <ResidueName3Isnt name3=“CYS” />
    </OperateOnCertainResidues>
    <LayerDesign name=“layer_core_boundary”
layer=“core_boundary” verbose=“False”
use_sidechain_neighbors=“True” />
  </TASKOPERATIONS>
  <FILTERS>
    <SheetTopology name=“filter_strand_pairing” topology=“1-
3.A.0;2-3.A.0” blueprint=“./EHEE.blueprint” />
    <CompoundStatement name=“compound_toplogy_filter” >
      <AND filter_name=“filter_strand_pairing” />
    </CompoundStatement>
    <TaskAwareScoreType name=“dslf_quality_check”
task_operations=“no_repack_non-disulf” scorefxn=“SFXN_FULLATOM”
score_type=“dslf_fal3” mode=“individual” threshold=“-0.27”
confidence=“1” />
    <DisulfideEntropy name=“entropy” lower_bound=“0”
tightness=“2” confidence=“0”/>
  ############### core assessment ###############
    <SecondaryStructureHasResidue name=“ss_contributes_core”
secstruct_fraction_threshold=“1.0”
res_check_task_operations=“layer_core_boundary”
required_restypes=“VILMFYW” nres_required_per_secstruct=“1”
filter_helix=“1” filter_sheet=“1” filter_loop=“0”
min_helix_length=“4” min_sheet_length=“3” min_loop_length=“1”
confidence=“1” />
  ##### verify presence of secondary structure #####
    <SecondaryStructureCount name=“count_SS_elements”
filter_helix_sheet=“True” num_helix=“1” num_sheet=“3”
num_helix_sheet=“4” min_helix_length=“6” min_sheet_length=“4”
min_loop_length=“2” />
    <CompoundStatement name=“sequence_quality_compound_filter”
>
      <AND filter_name=“ss_contributes_core” />
      <AND filter_name=“count_SS_elements” />
      <AND filter_name=“dslf_quality_check”/>
      <AND filter_name=“entropy” />
    </CompoundStatement>
  </FILTERS>
  <MOVERS>
  #### assess and record the secondary structure ####
    <Dssp name=“dssp” />
  #### design the protein mainchain ####
    <SetSecStructEnergies
name=“assign_secondary_structure_bonus” scorefxn=“SFXN_CENTROID”
blueprint=“./EHEE.blueprint” />
    <BluePrintBDR name=“build_mainchain”
scorefxn=“SFXN_CENTROID” use_abego_bias=“True”
blueprint=“./EHEE.blueprint” />
    <ParsedProtocol name=“mainchain_building_protocol” >
      <Add mover=“build_mainchain” />
      <Add mover=“dssp” />
    </ParsedProtocol>
    <LoopOver name=“mainchain_building_loop”
mover_name=“mainchain_building_protocol”
filter_name=“compound_toplogy_filter” iterations=“1000”
drift=“False” ms_whenfail=“FAIL_DO_NOT_RETRY” />
    <Disulfidize name=“disulfidizer” set1=“chain_A”
set2=“chain_A” min_disulfides=“2” max_disulfides=“3”
match_rt_limit=“2.0” score_or_matchrt=“true” max_disulf_score=”-
0.05” min_loop=“5” use_1_cys=“true”
keep_current_disulfides=“false”
include_current_disulfides=“false” use_d_cys=“false” />
    <FastDesign name=“fastdesign”
task_operations=“extra_rots,limitchi2,layer_all,no_design_disulf
,disallow_nonnative_ loop_sequences” scorefxn=“SFXN_FULLATOM”
clear_designable_residues=“0” repeats=“3”
ramp_down_constraints=“0” />
    <ParsedProtocol name=“build_mainchain_and_design_sequence”
>
      <Add mover_name=“assign_secondary_structure_bonus” />
      <Add mover=“mainchain_building_loop” />
      <Add mover=“dssp” />
      <Add mover_name=“disulfidizer” />
      <Add mover_name=“fastdesign” />
    </ParsedProtocol>
    <LoopOver name=“build_mainchain_and_design_sequence_loop”
mover_name=“build_mainchain_and_design_sequence”
filter_name=“sequence_quality_compound_filter” iterations=“1000”
drift=“False” ms_whenfail=“FAIL_DO_NOT_RETRY” />
  </MOVERS>
  <PROTOCOLS>
    <Add mover_name=“build_mainchain_and_design_sequence_loop”
/>
  </PROTOCOLS>
</ROSETTASCRIPTS>
```

Table 8 below shows an example blueprint file for designing an EHEE topology.

TABLE 8
SSPAIR 1-3.A.0; 2-3.A.0
HSSTRIPLET 1,3-1

| | | | |
|---|---|---|---|
| 1 | V | LE | . |
| 2 | V | EB | R |
| 0 | V | EB | R |
| 0 | V | EB | R |
| 0 | V | EB | R |
| 0 | V | EB | R |
| 0 | V | EB | R |
| 0 | V | EB | R |
| 0 | V | LG | R |
| 0 | V | LB | R |
| 0 | V | LB | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | LG | R |
| 0 | V | LB | R |
| 0 | V | EB | R |
| 0 | V | EB | R |
| 0 | V | EB | R |
| 0 | V | EB | R |
| 0 | V | EB | R |
| 0 | V | EB | R |
| 0 | V | EB | R |
| 0 | V | LE | R |
| 0 | V | LA | R |
| 0 | V | EB | R |
| 0 | V | EB | R |
| 0 | V | EB | R |
| 0 | V | EB | R |
| 0 | V | EB | R |
| 0 | V | EB | R |
| 0 | V | EB | R |
| 0 | V | LO | R |

Example Scripts and Inputs to Design Disulfide-Stapled Peptides

Table 9 below shows an example command line for running Rosetta™ scripts for designing di-sulfide stapled peptides:

TABLE 9

```
<path_to_Rosetta>/Rosetta/main/source/bin/rosetta_scripts.defaul
t.linuxgccrelease
  -in:file:s <arbitrary initial pdb file>
  -parser:protocol <Rosetta Scripts file>
  -out:file:s <output pdb file name>
  -run: preserve_header
```

Table 10 shows an example Rosetta™ scripts input file for designing di-sulfide stapled peptides:

```
TABLE 10
<ROSETTASCRIPTS>
<SCOREFXNS>
  ############## Define Score functions ###############
  <SFXN1 weights=“fldsgn_cen”>
    <Reweight scoretype=“cenpack” weight=“1.0” />
    <Reweight scoretype=“hbond_sr_bb” weight=“1.0” />
    <Reweight scoretype=“hbond_lr_bb” weight=“1.0” />
    <Reweight scoretype=“atom_pair_constraint” weight=“1.0” />
    <Reweight scoretype=“angle_constraint” weight=“1.0” />
    <Reweight scoretype=“dihedral_constraint” weight=“1.0” />
  </SFXN1>
  <SFXN_STD weights=“beta_july15.wts” />
</SCOREFXNS>
<TASKOPERATIONS>
</TASKOPERATIONS>
<FILTERS>
  <HelixKink name=“hk1” blueprint=“eeh.blueprint” />
  <SheetTopology name=“sf1” blueprint=“eeh.blueprint” />
  <SecondaryStructure name=“ss1” blueprint=“eeh.blueprint”
use_abego=“1” />
    <CompoundStatement name=“cs1”>
        <AND filter name=“ss1” />
        <AND filter name=“hk1” />
        <AND filter name=“sf1” />
    </CompoundStatement>
</FILTERS>
<MOVERS>
    <Dssp name=“dssp” />
    <SheetCstGenerator name=“sheet_new1”
cacb_dihedral_tolerance=“0.6” blueprint=“eeh.blueprint” />
    <SetSecStructEnergies name=“set_ssene1” scorefxn=“SFXN1”
blueprint=“eeh.blueprint” />
  <BluePrintBDR name=“topology_builder” use_abego_bias=“1”
scorefxn=“SFXN1” constraint_generators=“sheet_new1”
constraints_NtoC=“-1.0” blueprint=“eeh.blueprint” />
    <ParsedProtocol name=“build_dssp1” >
        <Add mover_name=“topology_builder” />
        <Add mover_name=“dssp” />
    </ParsedProtocol>
  <LoopOver name=“lover1” mover_name=“build_dssp1”
filter name=“cs1” iterations=“10” drift=“0”
ms_whenfail=“FAIL_DO_NOT_RETRY” />
  <ParsedProtocol name=“phase1” >
        <Add mover_name=“set_ssene1” />
        <Add mover_name=“lover1” />
    </ParsedProtocol>
  <ParsedProtocol name=“pp1”>
        <Add mover_name=“phase1” />
    </ParsedProtocol>
  #### Assemble the topology ####
  <LoopOver name=“lover2” mover_name=“pp1” filter_name=“cs1”
iterations=“10” drift=“0” ms_whenfail=“FAIL_DO_NOT_RETRY” />
    #### Add disulfides to the topology ####
  <Disulfidize name=“add_disulf” min_disulfides=“2”
max_disulfides=“2” max_disulf_score=“-0.20” match_rt_limit=“2”
min_loop=“5” />
    #### Design and Relax structures with disulfides in place
####
  <MultiplePoseMover name=“disulfidizer” >
      <SELECT>
      </SELECT>
      <ROSETTASCRIPTS>
        <SCOREFXNS>
          <SFXN_STD weights=“beta_july15.wts” />
        </SCOREFXNS>
        <FILTERS>
          <ResidueCount name=cys_count_1 residue_types=“CYS”
min_residue_count=4 confidence=1 />
        </FILTERS>
        <TASKOPERATIONS>
          <DisallowIfNonnative name=nocys resnum=0
disallow_aas=“C” />
          ############## select CYS residues
###############
          <OperateOnCertainResidues name=“no_design_disulf”
>
            <RestrictToRepackingRLT />
            <ResidueName3Is name3=“CYS” />
          </OperateOnCertainResidues>
          ########### layer selection for design ###########
          <LayerDesign name=“layer_all”
layer=“core_boundary_surface_Nterm_Cterm” verbose=“True”
use_sidechain_neighbors=“True” >
            <core>
              <all append=“M” />
            </core>
            <boundary>
            </boundary>
            <surface>
            </surface>
          </LayerDesign>
        </TASKOPERATIONS>
        <MOVERS>
          <FastDesign name=fdesign8 scorefxn=SFXN_STD
repeats=8 task_operations=layer_all, no_design_disulf,nocys
ramp_down_constraints=true>
            <MoveMap name=fdesign_mm>
              <Chain number=1 chi=true bb=true />
            </MoveMap>
          </FastDesign>
        </MOVERS>
        <PROTOCOLS>
          <Add filter=cys_count_1 />
          <Add mover=fdesign8 />
        </PROTOCOLS>
    </ROSETTASCRIPTS>
  </MultiplePoseMover>
</MOVERS>
<PROTOCOLS>
    <Add mover_name=“lover2” />
    <Add mover_name=“dssp” />
<Add mover_name=“add_disulf” />
<Add mover_name='7 disulfidizer” />
</PROTOCOLS>
</ROSETTASCRIPTS>
```

Table 11 below shows an example blueprint file for designing an EEH topology.

TABLE 11
SSPAIR 1-2.A.0

| | | | |
|---|---|---|---|
| 1 | V | LX | . |
| 0 | V | EB | R |
| 0 | V | EB | R |
| 0 | V | EB | R |
| 0 | V | EB | R |
| 0 | V | LG | R |
| 0 | V | LG | R |
| 0 | V | EB | R |
| 0 | V | EB | R |
| 0 | V | EB | R |
| 0 | V | EB | R |
| 0 | V | LB | R |
| 0 | V | LA | R |
| 0 | V | LB | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | HA | R |
| 0 | V | LX | R |

Example Scripts and Inputs to Design Peptides with Cyclic Heterochiral Topologies

Table 12 below shows an example command for running the example Rosetta™ Scripts XML file shown in Table 13 further below.

TABLE 12

```
<path_to_Rosetta>/Rosetta/main/source/bin/rosetta_scripts.defaul
t.linuxgccrelease
  -in:file:fasta <arbitrary initial fasta file>
  -parser:protocol <Rosetta Scripts file>
-out:file:s <output pdb file name>
```

Table 13 below shows an example Rosetta™ Scripts XML file.

```
TABLE 13
<ROSETTASCRIPTS>
<SCOREFXNS>
  <SFXN_STD weights= “beta_july15_cst.wts” />
  <SFXN_hbond_bb weights= “empty.wts” symmetric=0>
    <Reweight scoretype= hbond_sr_bb weight=1.17/>
    <Reweight scoretype= hbond_lr_bb weight=1.17/>
  </SFXN_hbond_bb>
</SCOREFXNS>
<TASKOPERATIONS>
</TASKOPERATIONS>
<FILTERS>
</FILTERS>
<MOVERS>
  <PeptideStubMover name=intial_stub reset=true>
    <Append resname=“GLY” />
    <Append resname=“ALA” />
    <Append resname=“ALA” />
    <Append resname=“ALA” />
    <Append resname=“ALA” />
    <Append resname=“ALA” />
    <Append resname=“ALA” />
    <Append resname=“ALA” />
    <Append resname=“ALA” />
    <Append resname=“ALA” />
    <Append resname=“ALA” />
    <Append resname=“GLY” />
    <Append resname=“VAL” />
    <Append resname=“VAL” />
    <Append resname=“DALA” />
    <Append resname=“DALA” />
    <Append resname=“DALA” />
    <Append resname=“DALA” />
    <Append resname=“DALA” />
    <Append resname=“DALA” />
    <Append resname=“DALA” />
    <Append resname=“DALA” />
    <Append resname=“DALA” />
    <Append resname=“DALA” />
    <Append resname=“ALA” />
    <Append resname=“GLY” />
  </PeptideStubMover>
  <DeclareBond name=peptide bond1 res1=1 atom1=“N” atom2=“C”
res2=26 add_termini=true />
  <SetTorsion name=torsion1>
    <Torsion residue=ALL torsion_name=omega angle=180.0 />
    <Torsion residue=1,12,13,14,25,26 torsion_name=rama
angle=rama_biased/>
    <Torsion residue=2,3,4,5,6,7,8,9,10,11 torsion_name=phi
angle=-64 .8/>
    <Torsion residue=2,3,4,5,6,7,8,9,10,11 torsion_name=psi
angle=-41 .0/>
    <Torsion residue=15,16,17,18,19,20,21,22,23,24
torsion_name=phi angle=64.8/>
    <Torsion residue=15,16,17,18,19,20,21,22,23,24
torsion_name=psi angle=41.0/>
  </SetTorsion>
  <GeneralizedKIC name=genkic1 closure_attempts=1000
name=genkic1 selector=“lowest_energy_selector”
stop_when_n_solutions_found=“50” stop_if_no_solution=500
selector_scorefunction=“SFXN_hbond_bb” >
    <AddResidue res_index=12 />
      <AddResidue res_index=13 />
    <AddResidue res_index=14 />
    <AddResidue res_index=15 />
    <AddResidue res_index=16 />
    <AddResidue res_index=17 />
    <AddResidue res_index=18 />
    <AddResidue res_index=19 />
    <AddResidue res_index=20 />
    <AddResidue res_index=21 />
    <AddResidue res_index=22 />
    <AddResidue res_index=23 />
    <AddResidue res_index=24 />
    <AddResidue res_index=25 />
    <AddResidue res_index=26 />
    <AddResidue res_index=1 />
    <SetPivots atom1 32 “CA” atom2=“CA” atom3=“CA” res1=12
res2=26 res3=1 />
    <CloseBond prioratom_res=26 prioratom=“CA” res1=26
atom1=“C” res2=1 atom2=“N” followingatom=“CA”
followingatom_res=1 angle1=116.199993 angle2=121.69997
bondlength=1.32865 randomize_flanking_torsions=false />
    <AddPerturber effect=“set_dihedral”>
<AddAtoms atom1=“C” res1=26 res2=1 atom2=“N” />
<AddValue value=180.0 />
    </AddPerturber>
    <AddPerturber effect=“randomize_alpha_backbone_by_rama”>
    <AddResidue index=12/>
      <AddResidue index=13 />
    <AddResidue index=14 />
    <AddResidue index=25/>
    <AddResidue index=26/>
    <AddResidue index=1/>
    </AddPerturber>
    <AddFilter type=“loop_bump_check” />
  <AddFilter type=“backbone_bin” bin_params_file=“ABBA”
residue=12 bin=“Bprime” />
    <AddFilter type=“backbone_bin” bin_params_file=“ABBA”
residue=13 bin=“A” />
    <AddFilter type=“backbone_bin” bin_params_file=“ABBA”
residue=14 bin=“B” />
    <AddFilter type=backbone_bin” bin_params_file=“ABBA”
residue=25 bin=“B” />
    <AddFilter type=“backbone_bin” bin_params_file=“ABBA”
residue=26 bin=“A” />
    <AddFilter type=“backbone_bin” bin_params_file=“ABBA”
residue=1 bin=“B” />
  </GeneralizedKIC>
  <CreateTorsionConstraint name=peptide_torsion_constraint>
    <Add res1=26 res2=26 res3=1 res4=1 atom1=“CA” atom2=“C”
atom3=“N” atom4=“CA” cst_func=“CIRCULARHARMONIC 3.141592654
0.005” />
    <Add res1=26 res2=26 res3=1 res4=1 atom1=“0” atom2=“C”
atom3=“N” atom4=“H” cst_func=“CIRCULARHARMONIC 3.141592654
0.005” />
  </CreateTorsionConstraint>
  <CreateAngleConstraint name=peptide_angle_constraints>
    <Add res1=26 atom1=“CA” res_center=26 atom_center=“C”
res2=1 atom2=“N” cst_func=“CIRCULARHARMONIC 2.02807247 0.005” />
    <Add res1=26 atom1=“C” res_center=1 atom center=“N” res2=1
atom2=“CA” cst_func=“CIRCULARHARMONIC 2.12406565 0.005” />
  </CreateAngleConstraint>
  <CreateDistanceConstraint name=N_To_C_dist_cst>
    <Add res1=26 res2=1 atom1=“C” atom2=“N” cst_func=“HARMONIC
1.32865 0.01” />
  </CreateDistanceConstraint>
  <Disulfidize name=“disulf” min_disulfides=“1”
max_disulfides=“1” max_disulf_score=“0.00” match_rt_limit=“1”
min_loop=“3” use_d_cys=“1” use_1_cys=“1” />
  <MultiplePoseMover name=“disulfidizer” >
    <SELECT>
    </SELECT>
    <ROSETTASCRIPTS>
      <SCOREFXNS>
        <SFXN_STD weights= “beta_july15_cst.wts” />
      </SCOREFXNS>
      <TASKOPERATIONS>
        <ReadResfile name=resfile_daa
filename=“./resfile1.txt” />
        <ReadResfile name=resfile_laa
filename=“./resfile2.txt” />
        <DisallowIfNonnative name=nocysgly resnum=0
disallow_aas=“CG” />
        <DisallowIfNonnative name=nocys resnum=0
disallow_aas=“C” />
        <LayerDesign name=laydesign make_pymol_script=0
use_sidechain_neighbors=1 />
        ############## select CYS residues ###############
        <OperateOnCertainResidues name=“no_repack_non-
disulf” >
          <PreventRepackingRLT/>
          <ResidueName3Isnt name3=“CYS” />
        </OperateOnCertainResidues>
        <OperateOnCertainResidues name=“no_design_disulf”
>
          <RestrictToRepackingRLT />
          <ResidueName3Is name3=“CYS,DCYS” />
        </OperateOnCertainResidues>
        ############ miscellaneous for design ############
        <LimitAromaChi2 name=“limitchi2” include_trp=“1”
/>
        ########### layer selection for design ###########
            ###Design with default layer design
settings###
            <LayerDesign name=“layer_all_noALA_Laa”
layer=“core_boundary_surface_Nterm_Cterm” verbose=“True”
use_sidechain_neighbors=“True” pore_radius=2.0 core=4.0
surface=1.8 >
          <core>
              <all append=“M” exclude=“A” />
          </core>
          <boundary>
              <all exclude=“A” />
          </boundary>
          <surface>
              <all exclude=“A” />
          </surface>
        </LayerDesign>
        <LayerDesign name=“layer_all_Laa”
layer=“core_boundary_surface_Nterm_Cterm” verbose=“True”
use_sidechain_neighbors=“True” pore_radius=2.0 core=4.5
surface=1.8 >
          <core>
              <all append=“M” />
          </core>
          <boundary>
              <all />
          </boundary>
          <surface>
              <all />
          </surface>
        </LayerDesign>
            ####Design with D-amino acid settings ###
        <LayerDesign name=“layer_all_noALA_Daa”
layer=“core_boundary_surface_Nterm_Cterm” verbose=“True”
use_sidechain_neighbors=“True” pore_radius=2.0 core=4.5
surface=1.8 >
          <core>
              <all
ncaa_append=“DPH,DLE,DIL,DPR,DVA,DTR,DTY” />
          </core>
          <boundary>
              <all
ncaa_append=“DVA,DTY,DTR,DTH,DSE,DPR,DPH,DLY,DLE,DIL,DGU,DAS,DAN
,DAR,DGN” />
          </boundary>
          <surface>
              <all
ncaa_append=“DTH,DSE,DPR,DLY,DHI,DGU,DAS,DAN,DAR,DGN” />
          </surface>
        </LayerDesign>
        <LayerDesign name=“layer_all_Daa”
layer=“core_boundary_surface_Nterm_Cterm” verbose=“True”
use_sidechain_neighbors=“True” pore_radius=2.0 core=4.0
surface=1.8 >
          <core>
              <all
ncaa_append=“DPH,DIL,DLE,DPR,DVA,DTR,DTY,DAL” />
          </core>
          <boundary>
              <all
ncaa_append=“DVA,DTY,DTR,DTH,DSE,DPR,DPH,DLY,DLE,DIL,DGU,DAS,DAN
,DAR,DAL,DGN” />
          </boundary>
          <surface>
              <all
ncaa_append=“DTH,DSE,DPR,DLY,DHI,DGU,DAS,DAN,DAR,DGN,DAL” />
          </surface>
        </LayerDesign>
      </TASKOPERATIONS>
      <FILTERS>
        <BuriedUnsatHbonds name=BuriedUnsat
scorefxn=SFXN_STD jump_number=0 cutoff=100 />
      </FILTERS>
      <MOVERS>
        <CreateTorsionConstraint
name=peptide_torsion_constraint>
          <Add res1=26 res2=26 res3=1 res4=1 atom1=“CA”
atom2=“C” atom3=“N” atom4=“CA” cst_func=“CIRCULARHARMONIC
3.141592654 0.005” />
          Add res1=26 res2=26 res3=1 res4=1 atom1=“0”
atom2=“C” atom3=“N” atom4=“H” cst_func=“CIRCULARHARMONIC
3.141592654 0.005” />
        </CreateTorsionConstraint>
        <CreateAngleConstraint
name=peptide_angle_constraints>
          <Add res1=26 atom1=“CA” res_center=26
atom_center=“C” res2=1 atom2=“N” cst_func=“CIRCULARHARMONIC
2.02807247 0.005” />
          <Add res1=26 atom1=“C” res_center=1
atom_center=“N” res2=1 atom2=“CA” cst_func=“CIRCULARHARMONIC
2.12406565 0.005” />
        </CreateAngleConstraint>
        <CreateDistanceConstraint name=N_To_C_dist_cst>
          <Add res1=26 res2=1 atom1=“C” atom2=“N”
cst_func=“HARMONIC 1.32865 0.01” />
        </CreateDistanceConstraint>
        <FastDesign name=fdesign2 scorefxn=SFXN_STD
repeats=2
task_operations=resfile_daa, layer_all_noALA_Daa,resfile_laa,laye
r_all_noALA_Daa,nocys,no_design_disulf,limitchi2
ramp_down_constraints=false>
          <MoveMap name=fdesign_mm>
              <Chain number=1 chi=true bb=true />
          </MoveMap>
        </FastDesign>
        <FastDesign name=fdesign6 scorefxn=SFXN_STD
repeats=6
task_operations=resfile_daa, layer_all_Daa,resfile_laa, layer_all_
Laa,nocys,no_design_disulf,limitchi2
ramp_down_constraints=false>
          <MoveMap name=fdesign_mm>
              <Chain number=1 chi=true bb=true />
          </MoveMap>
        </FastDesign>
        <DeclareBond name=peptide_bond1 res1=1 atom1=“N”
atom2=“C” res2=26 add_termini=true />
      </MOVERS>
      <PROTOCOLS>
        <Add mover=peptide_torsion_constraint />
        <Add mover=peptide_angle_constraints />
        <Add mover=N_To_C_dist_cst />
        <Add mover=fdesign2 />
        <Add mover=fdesign6 />
        <Add mover=peptide_bond1 />
        <Add filter=BuriedUnsat />
      </PROTOCOLS>
    </ROSETTASCRIPTS>
  </MultiplePoseMover>
</MOVERS>
<PROTOCOLS>
    <Add mover=intial_stub />
  <Add mover=torsion1 />
  <Add mover=peptide_bond1 />
  <Add mover=genkic1 />
  <Add mover=“disulf” />
  <Add mover_name=“disulfidizer” />
</PROTOCOLS>
</ROSETTASCRIPTS>
```

Table 14 below shows an example “resfile” for designing D-amino acids in the cyclic heterochiral topology. A resfile can be used to control behavior of the Rosetta™ packer, which optimizes sidechain conformations and/or identities given a fixed backbone. Note that, in this case, the following is intended for use with LayerDesign (as shown in Table 10 above), which will activate D-amino acid design at the “empty” positions.

TABLE 14
ALLAAwc
EX 1 EX 2
USE_INPUT_SC
start

| | | |
|---|---|---|
| 12 | A | EMPTY |
| 15 | A | EMPTY |
| 16 | A | EMPTY |
| 17 | A | EMPTY |
| 18 | A | EMPTY |
| 19 | A | EMPTY |
| 20 | A | EMPTY |
| 21 | A | EMPTY |
| 22 | A | EMPTY |
| 23 | A | EMPTY |
| 24 | A | EMPTY |

Table 15 below shows an example resfile for designing L-amino acids in the cyclic heterochiral topology. Note that the following is intended for use with LayerDesign (as shown in Table 10 above); the “RESET” commands are necessary to deactivate D-amino acid design at L-amino acid positions.

TABLE 15
start

| | | |
|---|---|---|
| 1 | A | RESET |
| 2 | A | RESET |
| 3 | A | RESET |
| 4 | A | RESET |
| 5 | A | RESET |
| 6 | A | RESET |
| 7 | A | RESET |
| 8 | A | RESET |
| 9 | A | RESET |
| 10 | A | RESET |
| 11 | A | RESET |
| 13 | A | RESET |
| 14 | A | RESET |
| 25 | A | RESET |
| 26 | A | RESET |

Example Computing Environment

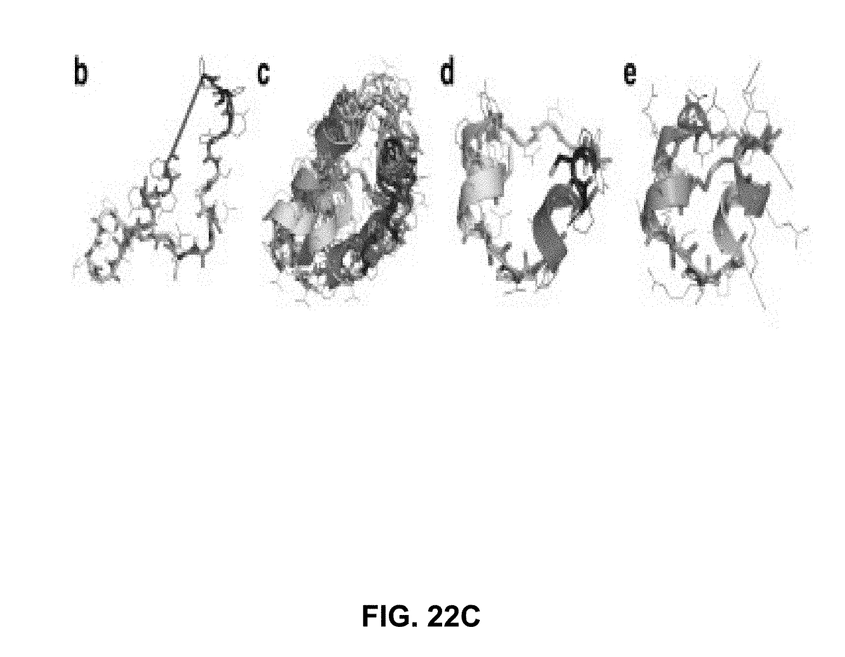

FIG. 23 is a block diagram of an example computing network. Some or all of the above-mentioned techniques disclosed herein, such as but not limited to techniques disclosed as part of and/or being performed by software, the Rosetta™ software suite, Rosetta™ Design, Rosetta™ applications, and/or other herein-described computer software and computer hardware, can be part of and/or performed by a computing device. For example, FIG. 23 shows protein design system 2302 configured to communicate, via network 2306, with client devices 2304a, 2304b, and 2304c and protein database 2308. In some embodiments, protein design system 2302 and/or protein database 2308 can be a computing device configured to perform some or all of the herein described methods and techniques, such as but not limited to, method 2000, the method shown in FIG. 21, the method shown in FIGS. 22A and 22B, and/or method 2500 and functionality described as being part of or related to Rosetta™. Protein database 2308 can, in some embodiments, store information related to and/or used by Rosetta™.

Network 2306 may correspond to a LAN, a wide area network (WAN), a corporate intranet, the public Internet, or any other type of network configured to provide a communications path between networked computing devices. Network 2306 may also correspond to a combination of one or more LANs, WANs, corporate intranets, and/or the public Internet.

Although FIG. 23 only shows three client devices 2304a, 2304b, 2304c, distributed application architectures may serve tens, hundreds, or thousands of client devices. Moreover, client devices 2304a, 2304b, 2304c (or any additional client devices) may be any sort of computing device, such as an ordinary laptop computer, desktop computer, network terminal, wireless communication device (e.g., a cell phone or smart phone), and so on. In some embodiments, client devices 2304a, 2304b, 2304c can be dedicated to problem solving/using the Rosetta™ software suite. In other embodiments, client devices 2304a, 2304b, 2304c can be used as general purpose computers that are configured to perform a number of tasks and need not be dedicated to problem solving/using Rosetta™. In still other embodiments, part or all of the functionality of protein design system 2302 and/or protein database 2308 can be incorporated in a client device, such as client device 2304a, 2304b, and/or 2304c.

Computing Environment Architecture

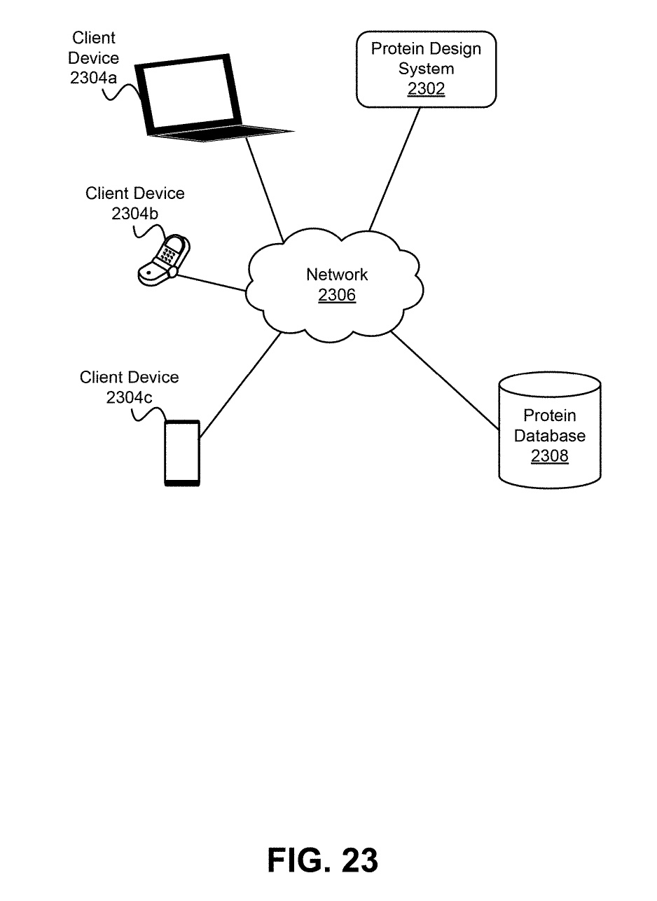

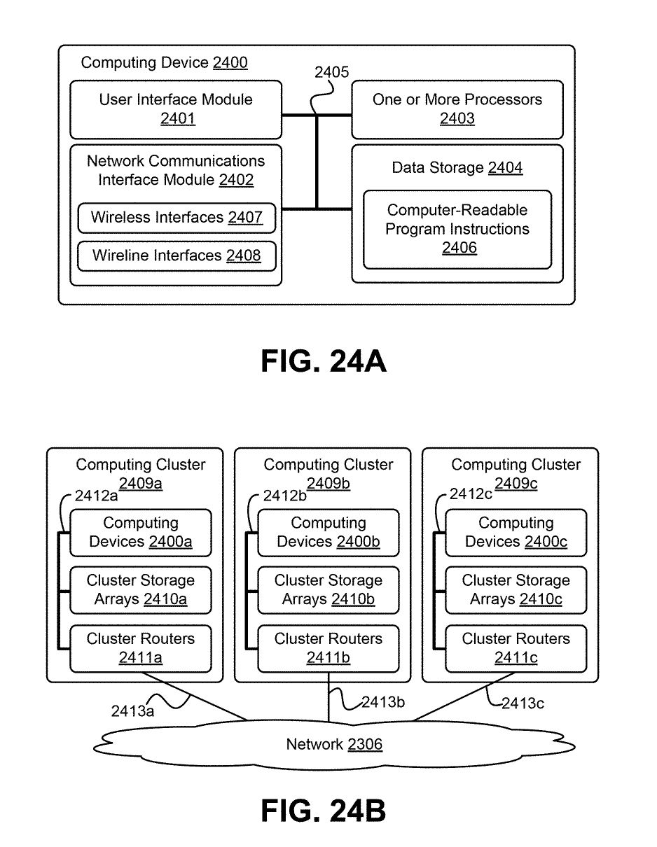

FIG. 24A is a block diagram of an example computing device (e.g., system) In particular, computing device 2400 shown in FIG. 24A can be configured to: include components of and/or perform one or more functions of protein design system 2302, client device 2304a, 2304b, 2304c, network 2306, and/or protein database 2308 and/or carry out part or all of any herein-described methods and techniques, such as but not limited to method 2000, the method shown in FIG. 21, the method shown in FIGS. 22A and 22B, and/or method 2500. Computing device 2400 may include a user interface module 2401, a network-communication interface module 2402, one or more processors 2403, and data storage 2404, all of which may be linked together via a system bus, network, or other connection mechanism 2405.

User interface module 2401 can be operable to send data to and/or receive data from external user input/output devices. For example, user interface module 2401 can be configured to send and/or receive data to and/or from user input devices such as a keyboard, a keypad, a touch screen, a computer mouse, a track ball, a joystick, a camera, a voice recognition module, and/or other similar devices. User interface module 2401 can also be configured to provide output to user display devices, such as one or more cathode ray tubes (CRT), liquid crystal displays (LCD), light emitting diodes (LEDs), displays using digital light processing (DLP) technology, printers, light bulbs, and/or other similar devices, either now known or later developed. User interface module 2401 can also be configured to generate audible output(s), such as a speaker, speaker jack, audio output port, audio output device, earphones, and/or other similar devices.

Network-communications interface module 2402 can include one or more wireless interfaces 2407 and/or one or more wireline interfaces 2408 that are configurable to communicate via a network, such as network 2306 shown in FIG. 23. Wireless interfaces 2407 can include one or more wireless transmitters, receivers, and/or transceivers, such as a Bluetooth transceiver, a Zigbee transceiver, a Wi-Fi transceiver, a WiMAX transceiver, and/or other similar type of wireless transceiver configurable to communicate via a wireless network. Wireline interfaces 2408 can include one or more wireline transmitters, receivers, and/or transceivers, such as an Ethernet transceiver, a Universal Serial Bus (USB) transceiver, or similar transceiver configurable to communicate via a twisted pair, one or more wires, a coaxial cable, a fiber-optic link, or a similar physical connection to a wireline network.

In some embodiments, network communications interface module 2402 can be configured to provide reliable, secured, and/or authenticated communications. For each communication described herein, information for ensuring reliable communications (i.e., guaranteed message delivery) can be provided, perhaps as part of a message header and/or footer (e.g., packet/message sequencing information, encapsulation header(s) and/or footer(s), size/time information, and transmission verification information such as CRC and/or parity check values). Communications can be made secure (e.g., be encoded or encrypted) and/or decrypted/decoded using one or more cryptographic protocols and/or algorithms, such as, but not limited to, DES, AES, RSA, Diffie-Hellman, and/or DSA. Other cryptographic protocols and/or algorithms can be used as well or in addition to those listed herein to secure (and then decrypt/decode) communications.

Processors 2403 can include one or more general purpose processors and/or one or more special purpose processors (e.g., digital signal processors, application specific integrated circuits, etc.). Processors 2403 can be configured to execute computer-readable program instructions 2406 contained in data storage 2404 and/or other instructions as described herein. Data storage 2404 can include one or more computer-readable storage media that can be read and/or accessed by at least one of processors 2403. The one or more computer-readable storage media can include volatile and/or non-volatile storage components, such as optical, magnetic, organic or other memory or disc storage, which can be integrated in whole or in part with at least one of processors 2403. In some embodiments, data storage 2404 can be implemented using a single physical device (e.g., one optical, magnetic, organic or other memory or disc storage unit), while in other embodiments, data storage 2404 can be implemented using two or more physical devices.

Data storage 2404 can include computer-readable program instructions 2406 and perhaps additional data. For example, in some embodiments, data storage 2404 can store part or all of data utilized by a protein design system and/or a protein database; e.g., protein designs system 2302, protein database 2308. In some embodiments, data storage 2404 can additionally include storage required to perform at least part of the herein-described methods and techniques and/or at least part of the functionality of the herein-described devices and networks.

FIG. 24B depicts a network 2306 of computing clusters 2409a, 2409b, 2409c arranged as a cloud-based server system in accordance with an example embodiment. Data and/or software for protein design system 2302 can be stored on one or more cloud-based devices that store program logic and/or data of cloud-based applications and/or services. In some embodiments, protein design system 2302 can be a single computing device residing in a single computing center. In other embodiments, protein design system 2302 can include multiple computing devices in a single computing center, or even multiple computing devices located in multiple computing centers located in diverse geographic locations.

In some embodiments, data and/or software for protein design system 2302 can be encoded as computer readable information stored in tangible computer readable media (or computer readable storage media) and accessible by client devices 2304a, 2304b, and 2304c, and/or other computing devices. In some embodiments, data and/or software for protein design system 2302 can be stored on a single disk drive or other tangible storage media, or can be implemented on multiple disk drives or other tangible storage media located at one or more diverse geographic locations.

FIG. 24B depicts a cloud-based server system in accordance with an example embodiment. In FIG. 24B, the functions of protein design system 2302 can be distributed among three computing clusters 2409a, 2409b, and 2409c. Computing cluster 2409a can include one or more computing devices 2400a, cluster storage arrays 2410a, and cluster routers 2411a connected by a local cluster network 2412a. Similarly, computing cluster 2409b can include one or more computing devices 2400b, cluster storage arrays 2410b, and cluster routers 2411b connected by a local cluster network 2412b. Likewise, computing cluster 2409c can include one or more computing devices 2400c, cluster storage arrays 2410c, and cluster routers 2411c connected by a local cluster network 2412c.

In some embodiments, each of the computing clusters 2409a, 2409b, and 2409c can have an equal number of computing devices, an equal number of cluster storage arrays, and an equal number of cluster routers. In other embodiments, however, each computing cluster can have different numbers of computing devices, different numbers of cluster storage arrays, and different numbers of cluster routers. The number of computing devices, cluster storage arrays, and cluster routers in each computing cluster can depend on the computing task or tasks assigned to each computing cluster.

In computing cluster 2409a, for example, computing devices 2400a can be configured to perform various computing tasks of protein design system 2302. In one embodiment, the various functionalities of protein design system 2302 can be distributed among one or more of computing devices 2400a, 2400b, and 2400c. Computing devices 2400b and 2400c in computing clusters 2409b and 2409c can be configured similarly to computing devices 2400a in computing cluster 2409a. On the other hand, in some embodiments, computing devices 2400a, 2400b, and 2400c can be configured to perform different functions.

In some embodiments, computing tasks and stored data associated with protein design system 2302 can be distributed across computing devices 2400a, 2400b, and 2400c based at least in part on the processing requirements of protein design system 2302, the processing capabilities of computing devices 2400a, 2400b, and 2400c, the latency of the network links between the computing devices in each computing cluster and between the computing clusters themselves, and/or other factors that can contribute to the cost, speed, fault-tolerance, resiliency, efficiency, and/or other design goals of the overall system architecture.

The cluster storage arrays 2410a, 2410b, and 2410c of the computing clusters 2409a, 2409b, and 2409c can be data storage arrays that include disk array controllers configured to manage read and write access to groups of hard disk drives. The disk array controllers, alone or in conjunction with their respective computing devices, can also be configured to manage backup or redundant copies of the data stored in the cluster storage arrays to protect against disk drive or other cluster storage array failures and/or network failures that prevent one or more computing devices from accessing one or more cluster storage arrays.

Similar to the manner in which the functions of protein design system 2302 can be distributed across computing devices 2400a, 2400b, and 2400c of computing clusters 2409a, 2409b, and 2409c, various active portions and/or backup portions of these components can be distributed across cluster storage arrays 2410a, 2410b, and 2410c. For example, some cluster storage arrays can be configured to store one portion of the data and/or software of protein design system 2302, while other cluster storage arrays can store a separate portion of the data and/or software of protein design system 2302. Additionally, some cluster storage arrays can be configured to store backup versions of data stored in other cluster storage arrays.

The cluster routers 2411a, 2411b, and 2411c in computing clusters 2409a, 2409b, and 2409c can include networking equipment configured to provide internal and external communications for the computing clusters. For example, the cluster routers 2411a in computing cluster 2409a can include one or more internet switching and routing devices configured to provide (i) local area network communications between the computing devices 2400a and the cluster storage arrays 2401a via the local cluster network 2412a, and (ii) wide area network communications between the computing cluster 2409a and the computing clusters 2409b and 2409c via the wide area network connection 2413a to network 2306. Cluster routers 2411b and 2411c can include network equipment similar to the cluster routers 2411a, and cluster routers 2411b and 2411c can perform similar networking functions for computing clusters 2409b and 2409b that cluster routers 2411a perform for computing cluster 2409a.

In some embodiments, the configuration of the cluster routers 2411a, 2411b, and 2411c can be based at least in part on the data communication requirements of the computing devices and cluster storage arrays, the data communications capabilities of the network equipment in the cluster routers 2411a, 2411b, and 2411c, the latency and throughput of local networks 2412a, 2412b, 2412c, the latency, throughput, and cost of wide area network links 2413a, 2413b, and 2413c, and/or other factors that can contribute to the cost, speed, fault-tolerance, resiliency, efficiency and/or other design goals of the moderation system architecture.

Example Methods of Operation

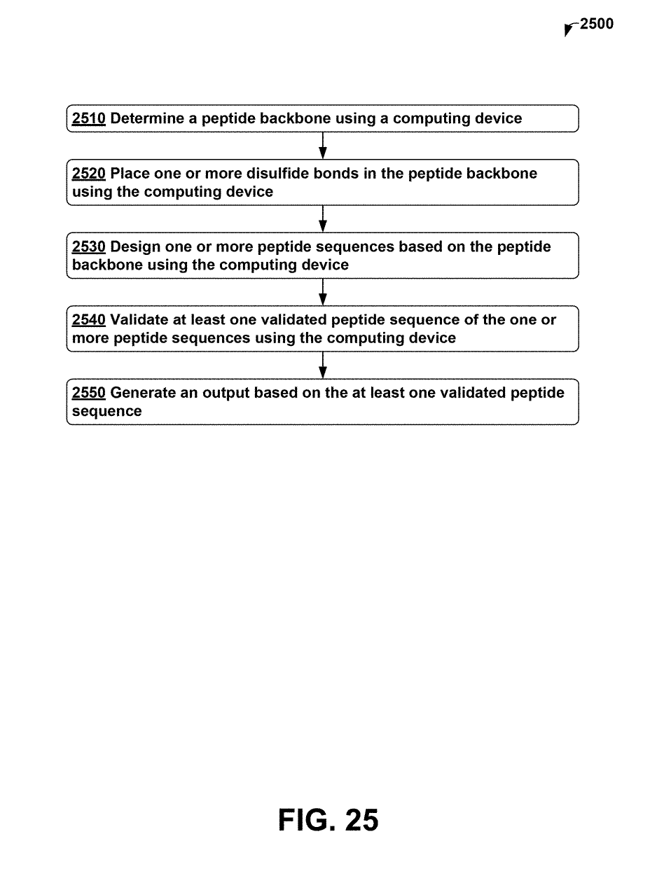

FIG. 25 is a flow chart of an example method 2500. Method 2500 can be carried out by a computing device, such as computing device 2400 described in the context of at least FIG. 24A. At least the embodiments of method 2500 mentioned below are discussed above; e.g., discussed above at least in the “Computational Techniques” section.

Method 2500 can begin at block 2510, where the computing device can determine a peptide backbone. In some embodiments, determining the peptide backbone can include determining the peptide backbone based on one or more protein topologies, such as In particular embodiments, the one or more protein topologies include one or more of: an HH topology, an HHH topology, an HEEE topology, a EHE topology, a EHEE topology, a EEH topology, a EEHE topology, a EEEH topology, and a EEEEEE topology, where an H of a topology denotes an α-helix and E of a topology denotes a β-strand. In other embodiments, determining the peptide backbone can include determining the peptide backbone based on a protein blueprint including a specification of a length of secondary structure in the peptide backbone, a specification of a connecting loop, and an ordering of elements in the peptide backbone. In still other embodiments, determining the peptide backbone can include: determining a protein blueprint for the peptide backbone; selecting one or more protein fragments based on the protein blueprint; and assembling the peptide backbone using the one or more protein fragments.

In even other embodiments, determining the peptide backbone can include assembling the peptide backbone using a generalized kinematic closure technique to close one or more atom chains in the peptide backbone. In some of these embodiments, assembling the peptide backbone using the generalized kinematic closure technique can include: determining an atom chain; determining one or more degree of freedom vectors based on conformation of the atom chain; and determining one or more candidate solutions to close the atom chain based on the one or more degree of freedom vectors. In other of these embodiments, assembling the peptide backbone using the generalized kinematic closure technique can further include perturbing the one or more degree of freedom vectors. In still other of these embodiments, assembling the peptide backbone using the generalized kinematic closure technique can further include: filtering the candidate solutions to close the atom chain based on one or more energy and/or geometric scores; determining whether a particular filtered candidate solution is a confirmed solution to close the atom chain based on a pre-selection protocol; after determining that the particular filtered candidate solution is a confirmed solution to close the atom chain, adding the particular filtered candidate solution to a confirmed solution list; and determining the peptide backbone based on the confirmed solution list.

At block 2520, the computing device can place one or more disulfide bonds in the peptide backbone.

At block 2530, the computing device can design one or more peptide sequences based on the peptide backbone. In some embodiments, designing the one or more peptide sequences based on the peptide backbone can include: determining the one or more peptide sequences using one or more design iterations, where a design iteration includes sidechain rotamer optimization and energy minimization; and filtering the one or more peptide sequences based on a residue energy score, a backbone quality score based on Ramachandran preference, and/or a disulfide geometry score. In some of these embodiments, validating at least one validated peptide sequence of the one or more peptide sequences includes validating the at least one validated peptide sequence using a fragment-based technique.

In other embodiments, the at least one validated peptide sequence can include a validated D-amino peptide sequence that has one or more D-amino acids. In some of these embodiments, the validated D-amino peptide sequence has one or more D-amino acids and one or more L-amino acids. In other of these embodiments, designing one or more peptide sequences includes determining one or more scores for the validated D-amino peptide sequence, and where the one or more scores include at least one of: a score for Ramachandran potential related to at least one of the one or more D-amino acids, a score for one or more torsion angles related to at least one of the one or more D-amino acids, and a score for sidechain conformations related to at least one of the one or more D-amino acids.

At block 2540, the computing device can validate at least one validated peptide sequence of the one or more peptide sequences. In some embodiments, validating at least one validated peptide sequence of the one or more peptide sequences can include: determining whether the at least one validated peptide sequence has a funnel-like energy landscape; after determining that the at least one validated peptide sequence has a funnel-like energy landscape, determining one or more trajectories associated with the at least one validated peptide sequence that has a funnel-like energy landscape using a molecular dynamics technique; determining whether the one or more trajectories are stable trajectories; and after determining that the one or more trajectories are stable trajectories, determining that the at least one molecular-dynamically validated peptide sequence.

In other embodiments, validating at least one validated peptide sequence of the one or more peptide sequences can include validating the at least one validated peptide sequence using a generalized kinematic closure validation technique. In some of these embodiments, validating the at least one validated peptide sequence using the generalized kinematic closure validation technique can include: performing a circular permutation of the at least one validated peptide sequence; constructing a linear peptide based on the at least one permuted validated peptide sequence; and validating the at least one permuted validated peptide sequence. In other of these embodiments, validating the at least one validated peptide sequence using the generalized kinematic closure validation technique can include: constructing one or more degree of freedom (DOF) vectors related to the at least one validated peptide sequence, where the one or more DOF vectors include one or more bond length, angle and/or torsion values; modify one or more of the bond length, angle and/or torsion values of the one or more DOF vectors based on one or more inputs; determining one or more candidate solutions for one or more loop closure equations that are based on the one or more DOF vectors; determining whether the one or more candidate solutions is a final solution of the one or more loop closure equations; and after determining that the one or more candidate solutions is the final solution of the one or more loop closure equations, validating at least a validated peptide sequence associated with the final solution of the one or more loop closure equations. In still other of these embodiments, determining whether the one or more candidate solutions is the final solution of the one or more loop closure equations can include: determining whether one or more pivots associated with a particular candidate solution are associated with one or more particular regions of Ramachandran space; and after determining that the one or more pivots associated with the particular candidate solution are associated with one or more particular regions of Ramachandran space: determining whether the particular solution has more hydrogen bonds that a predetermined number of hydrogen bonds, and after determining that the particular solution has more hydrogen bonds that the predetermined number of hydrogen bonds, determine that the particular solution is a final solution of the one or more loop closure equations.

At block 2550, the computing device and/or one or more other entities can generate an output based on the at least one validated peptide sequence. In some embodiments, the output related to the at least one validated peptide sequence can include a root-mean-square deviation (RMSD) value for atoms of the at least one validated peptide sequence. In other embodiments, the output related to the at least one validated peptide sequence can include an output related to a design of the at least one validated peptide sequence. In still other embodiments, the output related to the at least one validated peptide sequence includes an output related to a structure of the design of the at least one validated peptide sequence.

In still other embodiments, generating the output related to the on the at least one validated peptide sequence can include: generating a synthetic gene that is based on the at least one validated peptide sequence; expressing a particular protein in vivo using the synthetic gene; and purifying the particular protein. In particular of these embodiments, expressing the particular protein sequence in vivo using the synthetic gene includes expressing the particular protein sequence in one or more Escherichia coli that include the synthetic gene.

In some examples, at least a portion of method 2500 is performed by a computing device that includes: one or more data processors; and a computer-readable medium, configured to store at least computer-readable instructions that, when executed, cause the computing device to perform the at least a portion of method 2500. In particular of these examples, the computer-readable medium can include a non-transitory computer-readable medium.

In other examples, a computer-readable medium is provided, where the computer-readable medium is configured to store at least computer-readable instructions that, when executed by one or more processors of a computing device, cause the computing device to perform at least a portion of method 2500. In particular of these examples, the computer-readable medium can include a non-transitory computer-readable medium.

In still other examples, an apparatus is provided, where the apparatus can include means to perform at least a portion of method 2500.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The above definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The above description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure. The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The above detailed description describes various features and functions of the disclosed systems, devices, and methods with reference to the accompanying figures. In the figures, similar symbols typically identify similar components, unless context dictates otherwise. The illustrative embodiments described in the detailed description, figures, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the ladder diagrams, scenarios, and flow charts in the figures and as discussed herein, each block and/or communication may represent a processing of information and/or a transmission of information in accordance with example embodiments. Alternative embodiments are included within the scope of these example embodiments. In these alternative embodiments, for example, functions described as blocks, transmissions, communications, requests, responses, and/or messages may be executed out of order from that shown or discussed, including substantially concurrent or in reverse order, depending on the functionality involved. Further, more or fewer blocks and/or functions may be used with any of the ladder diagrams, scenarios, and flow charts discussed herein, and these ladder diagrams, scenarios, and flow charts may be combined with one another, in part or in whole.

A block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical functions or actions in the method or technique. The program code and/or related data may be stored on any type of computer readable medium such as a storage device including a disk or hard drive or other storage medium.

The computer readable medium may also include non-transitory computer readable media such as computer-readable media that stores data for short periods of time like register memory, processor cache, and random access memory (RAM). The computer readable media may also include non-transitory computer readable media that stores program code and/or data for longer periods of time, such as secondary or persistent long term storage, like read only memory (ROM), optical or magnetic disks, compact-disc read only memory (CD-ROM), for example. The computer readable media may also be any other volatile or non-volatile storage systems. A computer readable medium may be considered a computer readable storage medium, for example, or a tangible storage device. Moreover, a block that represents one or more information transmissions may correspond to information transmissions between software and/or hardware modules in the same physical device. However, other information transmissions may be between software modules and/or hardware modules in different physical devices.

Numerous modifications and variations of the present disclosure are possible in light of the above teachings.

We claim:

1. A method, comprising:
determining a peptide backbone conformation using a computing device;
placing zero or more disulfide bonds in the peptide backbone conformation using the computing device;
designing one or more peptide sequences based on the peptide backbone conformation using the computing device;
validating at least one peptide sequence of the one or more peptide sequences using the computing device; and
generating an output based on the at least one validated peptide sequence.

2. The method of claim 1, wherein determining the peptide backbone conformation comprises determining the peptide backbone conformation based on one or more protein topologies that comprise one or more of: an HH topology, an HHH topology, an HEEE topology, a EHE topology, a EHEE topology, a EEH topology, a EEHE topology, a EEEH topology, and a EEEEEE topology, where an H of a topology denotes an α-helix and E of a topology denotes a β-strand.

3. The method of claim 1, wherein determining the peptide backbone conformation comprises determining the peptide backbone conformation based on a protein blueprint comprising a specification of a length of secondary structure in the peptide backbone conformation, a specification of a connecting loop, and an ordering of elements in the peptide backbone conformation.

4. The method of claim 1, wherein determining the peptide backbone conformation comprises:
determining a protein blueprint for the peptide backbone conformation;
selecting one or more protein fragments based on the protein blueprint; and
assembling the peptide backbone conformation using the one or more protein fragments.

5. The method of claim 1, wherein determining the peptide backbone conformation comprises assembling the peptide backbone conformation using a generalized kinematic closure technique to close one or more atom chains in the peptide backbone conformation by at least:
determining an atom chain;
determining one or more degree of freedom vectors based on conformation of the atom chain; and
determining one or more candidate solutions to close the atom chain based on the one or more degree of freedom vectors.

6. The method of claim 5, wherein assembling the peptide backbone conformation using the generalized kinematic closure technique further comprises perturbing the one or more degree of freedom vectors.

7. The method of claim 5, wherein assembling the peptide backbone conformation using the generalized kinematic closure technique further comprises:
filtering the candidate solutions to close the atom chain based on one or more energy and/or geometric scores;
determining whether a particular filtered candidate solution is a confirmed solution to close the atom chain based on a pre-selection protocol;
after determining that the particular filtered candidate solution is a confirmed solution to close the atom chain, adding the particular filtered candidate solution to a confirmed solution list; and
determining the peptide backbone conformation based on the confirmed solution list.

8. The method of claim 1, wherein designing the one or more peptide sequences based on the peptide backbone conformation comprises:
determining the one or more peptide sequences using one or more design iterations, wherein a design iteration includes sidechain identity, rotamer optimization, and energy minimization; and
filtering the one or more peptide sequences based on a residue energy score, a backbone quality score based on Ramachandran conformational preference, and/or a disulfide geometry score.

9. The method of claim 1, wherein validating the at least one peptide sequence of the one or more peptide sequences comprises validating the at least one peptide sequence using a fragment-based technique.

10. The method of claim 1, wherein validating the at least one peptide sequence of the one or more peptide sequences comprises:
determining whether the at least one peptide sequence has a funnel-like energy landscape;
after determining that the at least one peptide sequence has a funnel-like energy landscape, determining one or more trajectories associated with the at least one peptide sequence that has a funnel-like energy landscape using a molecular dynamics technique;
determining whether the one or more trajectories are stable trajectories; and
after determining that the one or more trajectories are stable trajectories, determining that the at least one peptide sequence is molecular-dynamically validated.

11. The method of claim 1, wherein validating at least one peptide sequence of the one or more peptide sequences comprises validating the at least one peptide sequence using a generalized kinematic closure validation technique.

12. The method of claim 11, wherein validating the at least one peptide sequence using the generalized kinematic closure validation technique comprises:
performing a circular permutation of the at least one peptide sequence;
constructing a linear peptide based on the at least one permuted peptide sequence; and
validating the at least one permuted peptide sequence.

13. The method of claim 11, wherein validating the at least one peptide sequence using the generalized kinematic closure validation technique comprises:
constructing one or more degree of freedom (DOF) vectors related to the at least one peptide sequence, wherein the one or more DOF vectors comprise one or more bond length, angle and/or torsion values;
modify one or more of the bond length, angle and/or torsion values of the one or more DOF vectors based on one or more inputs;
determining one or more candidate solutions for one or more loop closure equations that are based on the one or more DOF vectors;
determining whether the one or more candidate solutions is a final solution of the one or more loop closure equations; and
after determining that the one or more candidate solutions is the final solution of the one or more loop closure equations, validating at least one peptide sequence associated with the final solution of the one or more loop closure equations.

14. The method of claim 13, wherein determining whether the one or more candidate solutions is the final solution of the one or more loop closure equations comprises:
determining whether one or more pivots associated with a particular candidate solution are associated with one or more particular regions of Ramachandran space; and
after determining that the one or more pivots associated with the particular candidate solution are associated with one or more particular regions of Ramachandran space:
determining whether the particular solution has more hydrogen bonds that a predetermined number of hydrogen bonds, and
after determining that the particular solution has more hydrogen bonds that the predetermined number of hydrogen bonds, determine that the particular solution is a final solution of the one or more loop closure equations.

15. A computing device, comprising:
one or more processors; and
a non-transitory computer-readable medium, configured to store at least computer-readable instructions that, when executed by the one or more processors, cause the computing device to perform functions comprising the method steps of claim 1.

16. A non-transitory computer-readable medium, configured to store at least computer-readable instructions that, when executed by one or more processors of a computing device, cause the computing device to perform functions comprising the method steps of claim 1.

17. A non-naturally occurring polypeptide comprising
(a) 2-6 secondary structure domains, wherein each secondary structure domain is either a β-sheet (E domain) of between 4-9 amino acid residues in length, or an α-helix (H domain) of between 4-15 amino acid residues in length; and
(b) a loop of 2-5 amino acid residues in length connecting adjacent secondary structure domains;
wherein the polypeptide is between 15-50 amino acid residues in length.

18. An isolated nucleic acid encoding the polypeptide of claim 17.

19. A recombinant expression vector comprising the isolated nucleic acid of claim 18 operatively linked to a promoter.

20. A recombinant host cell comprising the recombinant expression vector of claim 19.
